SEQUENCE LISTING (4 sequences)

SEQ ID NO: 1 - protein, 31 aa, Artificial Sequence
  Synthetic polypeptide
HAEGTFTSDVSSYLEGQAAKEFIAWLVKGRG

SEQ ID NO: 2 - protein, 31 aa, Artificial Sequence
  Synthetic polypeptide
  misc_feature (1)..(2): Xaa can be any naturally occurring amino acid
  MISC_FEATURE (10)..(10): VaI or Leu
  MISC_FEATURE (12)..(12): Ser, Lys or Arg
  MISC_FEATURE (13)..(13): Tyr or GIn
  MISC_FEATURE (14)..(14): Leu or Met
  MISC_FEATURE (16)..(16): GIy, Glu or Aib
  MISC_FEATURE (17)..(17): GIn, Glu, Lys or Arg
  MISC_FEATURE (19)..(19): Ala or VaI
  MISC_FEATURE (21)..(21): Glu or Leu
  MISC_FEATURE (24)..(24): Ala, Glu or Arg
  MISC_FEATURE (27)..(27): VaI or Lys
  MISC_FEATURE (28)..(28): Lys, Glu, Asn or Arg
  MISC_FEATURE (29)..(29): GIy or Aib
  MISC_FEATURE (30)..(30): Arg, GIy or Lys, or is absent
  MISC_FEATURE (31)..(31): GIy, Ala, Glu, Pro, Lys, or is absent
XXEGTFTSDXSXXXEXXAXRXFIXWLXXXXX

SEQ ID NO: 3 - protein, 31 aa, Artificial Sequence
  Synthetic polypeptide
  MOD_RES (2)..(2): Aib
HXEGTFTSDVSSYLEGQAAKEFIAWLVRGRG

SEQ ID NO: 4 - protein, 31 aa, Artificial Sequence
  Synthetic polypeptide
HAEGTFTSDVSSYLEGQAAKEFIAWLVRGRG

(19) United States
(12) Patent Application Publication
(10) Pub. No.: US 2024/0247042 A1
(43) Pub. Date: Jul. 25, 2024

(54) GLUCAGON LIKE PEPTIDE COMPOUNDS

(71) Applicant: Novartis AG, Basel (CH)

(72) Inventors: Valerie BEAULIEU, North Reading, MA (US); Keith GALYAN, Lake Villa, IL (US); Jinhai GAO, Belmont, MA (US); Carol GINSBURG-MORAFF, Cambridge, MA (US); Pankaj JAIN, Belmont, MA (US); Jovita MARCINKEVICIENE, Lexington, MA (US); Walter MICHAEL, Watertown, MA (US); Muneto MOGI, Waltham, MA (US); Martin MARRO, Newton, MA (US); Sejal PATEL, Lexington, MA (US); Scott PLUMMER, Somerville, MA (US); Alok SINGH, Sudbury, MA (US); Aimee USERA, Winchester, MA (US)

(21) Appl. No.: 18/556,461

(22) PCT Filed: Apr. 20, 2022
(86) PCT No.: PCT/IB2022/053698

§ 371 Date: Oct. 20, 2023

(30) Foreign Application Priority Data
Apr. 21, 2021 (EP) 21169757.8

(51) Int. Cl. C07K 14/605; A61K 38/00; A61K 47/54; A61P 3/04

(52) U.S. Cl. CPC C07K 14/605; A61K 47/542; A61P 3/04; A61K 38/00

(57) ABSTRACT

Provided herein are novel compounds comprising a GLP-1 compound and a fatty acid or fatty acid derivative, the manufacture of said novel compounds and the use thereof.

### TECHNICAL FIELD

Provided herein are novel compounds comprising a GLP-1 compound and a fatty acid or fatty acid derivative, the manufacture of said novel compounds and the use thereof. These novel compounds exhibit favorable pharmacological efficacies.

### BACKGROUND

Glucagon-like peptide (GLP) 1 (GLP1) agonists belong to an important class of therapeutically effective compounds. GLP1 agonists are typically used in the treatment of diabetes type 2. Various approaches have been used for modifying the structure of such glucagon-like peptide 1 (GLP1) compounds in order to prevent a rapid biodegradation to provide a satisfactory duration of action in vivo and to improve tolerability.

For example, WO 2006/097537 (Novo Nordisk) describes GLP1 compounds having a modification of at least one non-natural amino acid residue in positions 7 and/or 8 relative to the sequence GLP-1 (7-37) (SEQ ID NO:1) which is acylated with a moiety to the lysine residue in position 26, wherein said moiety comprises at least two acidic groups.

WO 2015/200078 (Novartis) discloses a conjugate comprising a biomolecule such as GDF15 being linked to a fatty acid via a linker. The corresponding conjugates may be useful in the treatment or prevention of metabolic diseases or disorders.

### SUMMARY

Provided herein are compounds comprising a GLP-1 or GLP-1 analogue, covalently bound, optionally via a linker, to a compound of formula (i) or a pharmaceutically acceptable salt thereof:

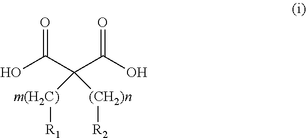

wherein,
R₁ and R₂ are independently selected from CH₃, OH, CO₂H, CH═CH₂ and C≡CH;
n and m are each an integer independently selected from 5 to 30;
and wherein the compound of Formula (i) is covalently bound through one of its CO₂H groups.

The compounds described herein may typically act as agonists of the Glucagon-like Peptide 1 Receptor (GLP1R). Accordingly, these compounds may be useful in the treatment of diseases or disorders including but not limited to: metabolic diseases, disorders and conditions, such as obesity, type 2 diabetes mellitus, insulin resistance, hyperinsulinemia, glucose intolerance, hyperglycemia, one or more diabetic complications (including but not limited to chronic kidney disease), diabetic nephropathy, dyslipidemia, cardiovascular disease and neuropathy. The compounds may also be potentially useful in the treatment of progressive liver disease and neuropathies.

### Definitions

The term “peptide” as used herein means a compound composed of at least five amino acids connected by peptide bonds. The amino acids may be naturally occurring amino acids as well as non-naturally occurring amino acids. Some peptides may be composed of all naturally occurring amino acids. Some peptides may be composed of all non-naturally occurring amino acids. Some peptides may be composed of a mixture of naturally occurring amino acids and non-naturally occurring amino acids.

The term “naturally occurring” refers to materials which are found in nature and are not manipulated by man. Similarly, “non-naturally occurring,” “un-natural,” and the like, as used herein, refers to a material that is not found in nature or that has been structurally modified or synthesized by man.

When used in connection with amino acids, the term “naturally occurring” usually refers to 22 conventional amino acids, such as: alanine (A or Ala), cysteine (C or Cys), cystine (CySS), aspartic acid (D or Asp), glutamic acid (E or Glu), phenylalanine (F or Phe), glycine (G or Gly), histidine (H or His), isoleucine (I or lie), lysine (K or Lys), leucine (L or Leu), methionine (M or Met), asparagine (N or Asn), proline (P or Pro), 4-hydroxyproline (O or Hyp), glutamine (Q or Gln), arginine (R or Arg), serine (S or Ser), threonine (T or Thr), valine (V or Val), tryptophan (W or Trp), and tyrosine (Y or Tyr)).

The terms “non-naturally occurring amino acid,” “non-natural amino acid,” and “unnatural amino acid,” as used herein, are interchangeably intended to represent amino acid structures that cannot be generated biosynthetically in any organism using unmodified or modified genes from any organism, whether the same or different. These include, but are not limited to, modified amino acids and/or amino acid analogues that are not one of the above 22 naturally occurring amino acids.

Examples of non-natural amino acid are γ-carboxyglutamate, ornithine, phosphoserine, the D-amino acids such as D-alanine and D-glutamine. Synthetic non-natural amino acids comprise amino acids manufactured by chemical synthesis, i.e., D-isomers of the amino acids such as D-alanine and D-leucine, Aib (α-aminoisobutyric acid), Abu (α-aminobutyric acid), Tle (tert-butylglycine), 3-aminomethyl benzoic acid, anthranilic acid, des-amino-histidine, the beta analogs of amino acids such as β-alanine etc., e.g., D-histidine, desamino-histidine, 2-amino-histidine, beta-hydroxy-histidine, homohistidine, N^{α}-acetyl-histidine, α-fluoromethyl-histidine, α-methyl-histidine, 3-pyridylalanine, 2-pyridylalanine or 4-pyridylalanine, (1-aminocyclopropyl) carboxylic acid, (1-aminocyclobutyl) carboxylic acid, (1-aminocyclopentyl) carboxylic acid, (1-aminocyclohexyl) carboxylic acid, (1-aminocycloheptyl) carboxylic acid, or (1-aminocyclooctyl) carboxylic acid.

All amino acids for which the optical isomer is not stated are to be understood to mean the L-isomer.

The term “analogue” or “analog” of a peptide as used herein means a modified peptide, wherein one or more amino acid residues of the peptide have been substituted once or more times by another amino acid residue and/or wherein one or more amino acid residues have been deleted from the peptide and/or wherein one or more amino acid residues have been added to the peptide. Such addition or deletion of amino acid residues can take place at the any place within the peptide. For example, such addition or deletion of amino acid residues can take place within the N-terminal part of the peptide and/or at the C-terminal part of the peptide.

The term “GLP-1” as used herein means GLP-1 (7-37) (SEQ ID NO:1).

The term “GLP-1 analogue” as used herein refers to an analogue of the GLP-1 (7-37) as defined above, wherein the term “analogue” is as defined above. For example [Arg³⁴]GLP-1 (7-37)Lys designates a GLP-1 (7-37) analogue wherein the naturally occurring lysine at position 34 of GLP-1 (7-37) has been substituted with arginine and wherein a lysine has been added to the terminal amino acid residue, i.e., to the Gly³⁷.

### FURTHER EMBODIMENTS

Another embodiment provides a compound of formula (i) according to the previous embodiment, which is a compound of formula (I) or a pharmaceutically acceptable salt thereof:

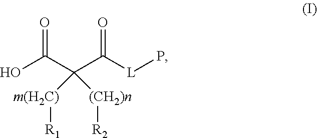

wherein,
R₁ and R₂ are independently selected from CH₃, OH, CO₂H, CH═CH₂ and C≡CH;
n and m are each an integer independently selected from 5 to 30;
L is an optional linker and P is a GLP-1 or GLP-1 analogue.

Another embodiment provides a compound of formula (I) or a pharmaceutically acceptable salt thereof in accordance with the previous embodiment, wherein the GLP-1 or GLP-1 analogue (P) is bound to the optional linker (L) via an NH group.

Another embodiment provides a compound of formula (I) or a pharmaceutically acceptable salt thereof in accordance with the previous embodiments,

wherein the linker (L) is selected from:

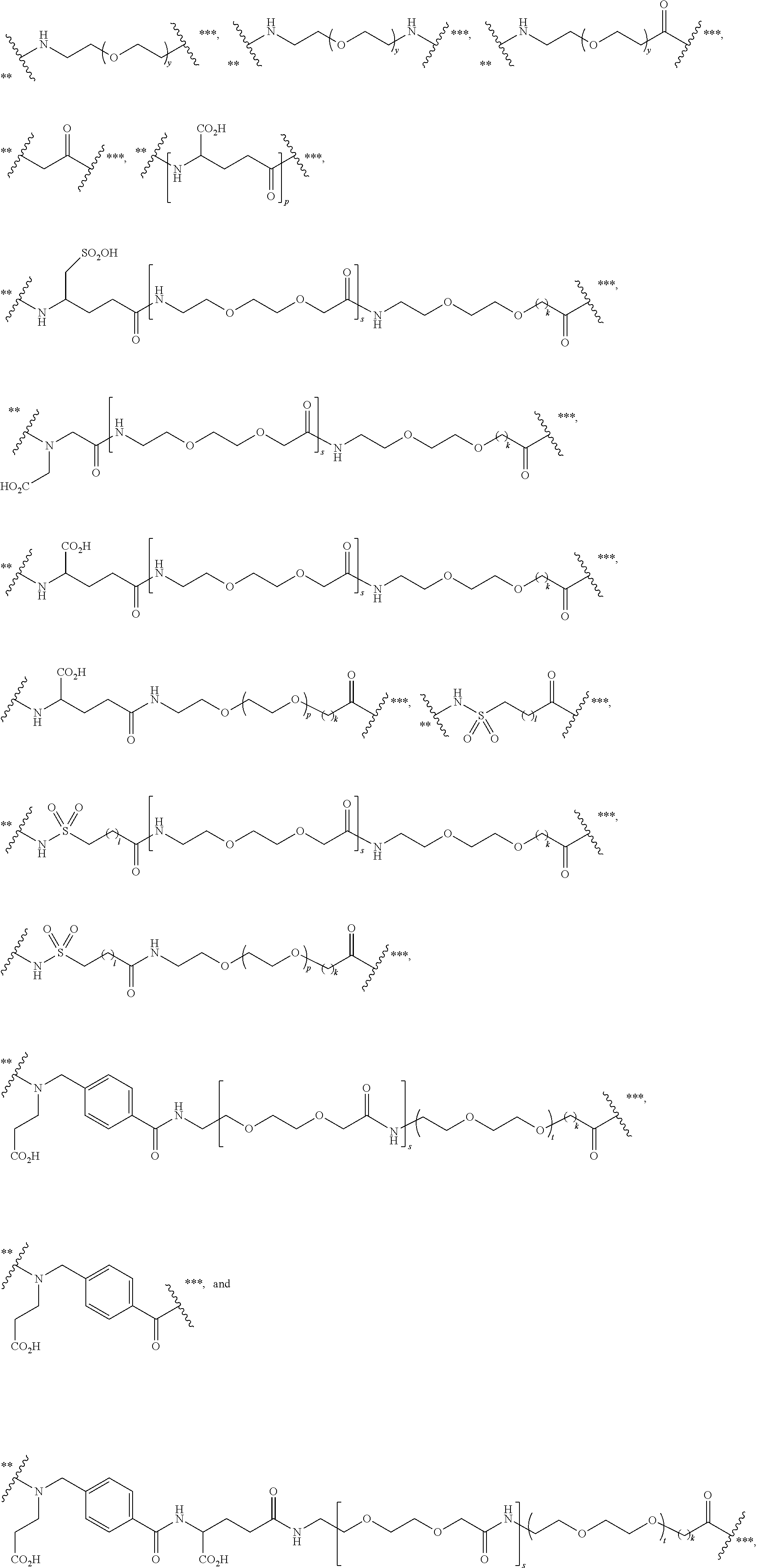

wherein,
y is an integer selected from 1 to 36;
l is 0, 1, 2, 3, 4, 5 or 6;
k is 1, 2 or 3;
s is 0, 1, 2 or 3;
t is 0, 1, 2, 3 or 4;
p is 1, 2, 3, 4, 5, 6, 7, 8, 9, 10, 11, 12, 13, 14, 15, 16, 17, 18, 19, 20, 21, 22 or 23;
wherein the wavy line marked ** indicate the attachment to the CO-group of formula (I) and wherein the wavy line marked *** indicate the attachment to group P.

Another embodiment provides a compound of formula (I) or a pharmaceutically acceptable salt thereof in accordance to the previous embodiment, wherein

y is an integer selected from 1 to 36;
l is 2, 3, 4 or 5;
k is 1 or 2;
s is 0, 1 or 2;
t is 0, 1, 2 or 3; and
p is 1, 2, 3, 4, 7, 11 or 23.

Another embodiment provides a compound of formula (I) or a pharmaceutically acceptable salt thereof in accordance to the previous embodiment, wherein y is an integer selected from 1 to 36;

l is 2, 3, 4 or 5;
k is 1 or 2;
s is 0, 1 or 2;
t is 0 or 1; and
p is 1, 2, 3, 4 or 11.

Another embodiment provides a compound of formula (I) or a pharmaceutically acceptable salt thereof in accordance to the previous embodiment, wherein the linker (L) is selected from:

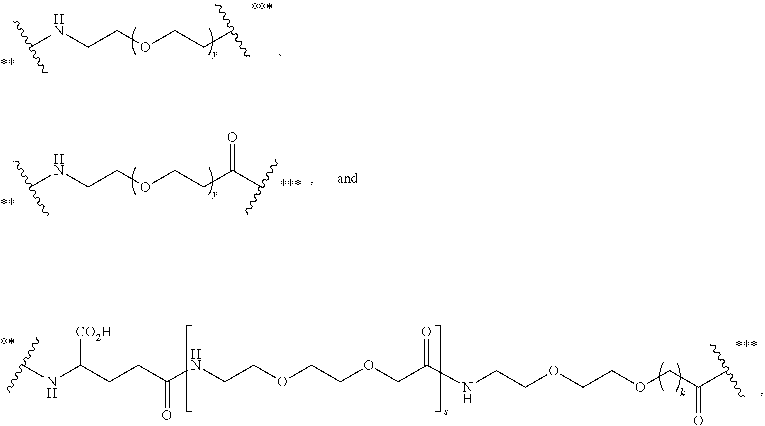

wherein
y is an integer selected from 1 to 36,
s is 1 and k is 1, and
wherein the wavy line marked ** indicate the attachment to the CO-group of formula (I) and
wherein the wavy line marked *** indicate the attachment to group P.

Another embodiment provides compound of formula (I) or a pharmaceutically acceptable salt thereof according to the previous embodiment, wherein L is selected from:

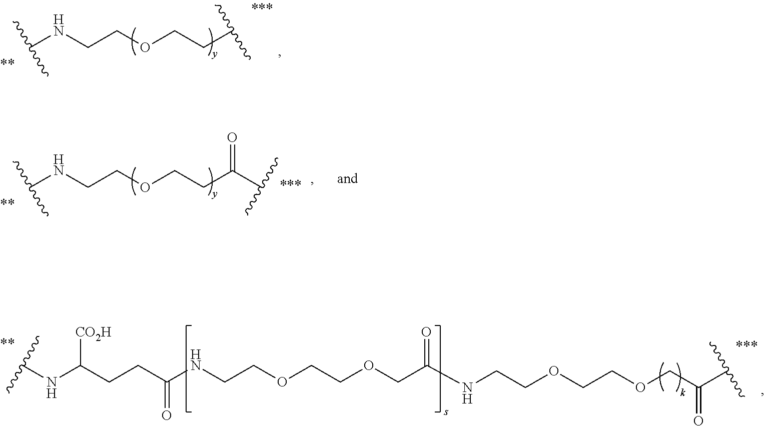

wherein:
y is an integer selected from 1 to 36,
s is 0, 1 or 2 and k is 1, 2 or 3, and
the wavy line marked ** indicate the attachment to the CO-group of formula (I), and
the wavy line marked *** indicate the attachment to group P.

Another embodiment provides a compound of formula (I) or a pharmaceutically acceptable salt thereof in accordance to the previous embodiment, wherein the linker (L) is selected from:

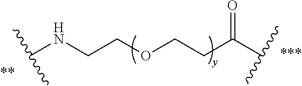

wherein y is an integer selected from 1 to 36.

Another embodiment provides a compound of formula (I) or a pharmaceutically acceptable salt thereof in accordance to the previous embodiment, wherein the carbon atom of the C(O) group of said linker is attached to the nitrogen atom of an NH group of a lysine residue of the GLP-1 or GLP-1 analogue.

Another embodiment provides a compound of formula (I) or a pharmaceutically acceptable salt thereof in accordance to the previous embodiments, wherein R₁ and R₂ are independently selected from CH₃, OH and CO₂H.

Another embodiment provides a compound of formula (I) in accordance to the previous embodiments, which is a compound of formula (II) or a pharmaceutically acceptable salt thereof,

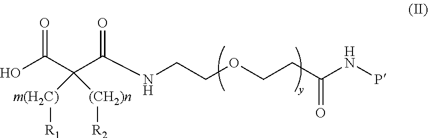

wherein
NH—P′ represents a group P (i.e., a GLP-1 or GLP-1 analogue) which is attached via a NH-moiety to the linker L;
R₁ and R₂ are independently selected from CH₃, OH and CO₂H;
n and m are each an integer independently selected from 5 to 30;
and
y is an integer selected from 1 to 36.

In one embodiment, the group P (i.e., a GLP-1 or GLP-1 analogue) corresponds to P′—NH₂, i.e. a P group with a free —NH₂ group, which is part of an amino acid side chain, and P is attached to the linker L via said —NH group.

Another embodiment provides a compound of formula (II) as defined herein above, wherein R₁ and R₂ are independently selected from CO₂H and CH₃.

Another embodiment provides a compound of formula (II) as defined herein above, wherein n and m are each an integer independently selected from 5 to 20.

Another embodiment provides a compound of formula (II) as defined herein above, wherein n and m are each an integer independently selected from 10, 11, 13 and 14.

Another embodiment provides a compound of formula (II) as defined herein above, wherein

R₁ is CO₂H and R₂ is CH₃; n is 10 and m is 10;
R₁ is CO₂H and R₂ is CO₂H; n is 10 and m is 10;
R₁ is CO₂H and R₂ is CO₂H; n is 10 and m is 11;
R₁ is CO₂H and R₂ is CO₂H; n is 10 and m is 13; or
R₁ is CO₂H and R₂ is CO₂H; n is 10 and m is 14.

Another embodiment provides a compound of formula (II) as defined herein, which is a compound of formula (III) or pharmaceutically acceptable salt thereof,

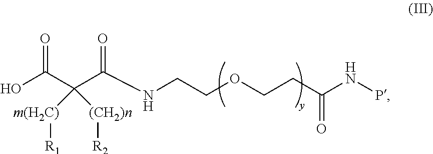

wherein R₁ is CO₂H and R₂ is CH₃.

Another embodiment provides a compound of formula (III) as defined herein which is a compound of formula (IIIa) or a compound of formula (IIIb) or pharmaceutically acceptable salt thereof,

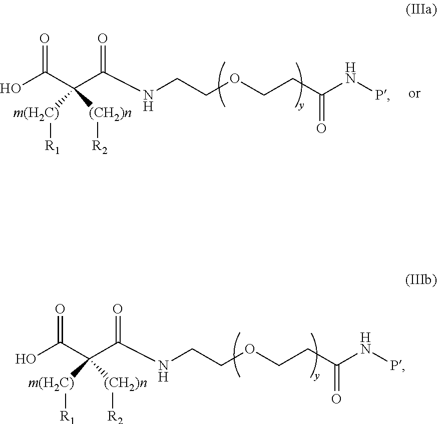

wherein R₁ is CO₂H and R₂ is CH₃.

Another embodiment provides a compound of formula (II) as defined herein, which is a compound of formula (IV) or a pharmaceutically acceptable salt thereof,

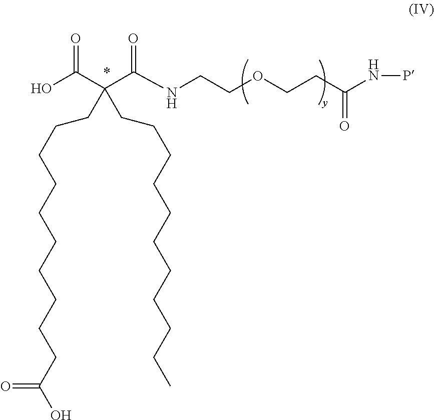

wherein the compound is present as a racemate, or as a stereochemically enriched mixture, or is stereochemically pure in respect of the carbon atom marked *.

Another embodiment provides a compound of formula (IV) as defined herein before, which is a compound of formula (IVa) or a compound of formula (IVb) or a pharmaceutically acceptable salt thereof,

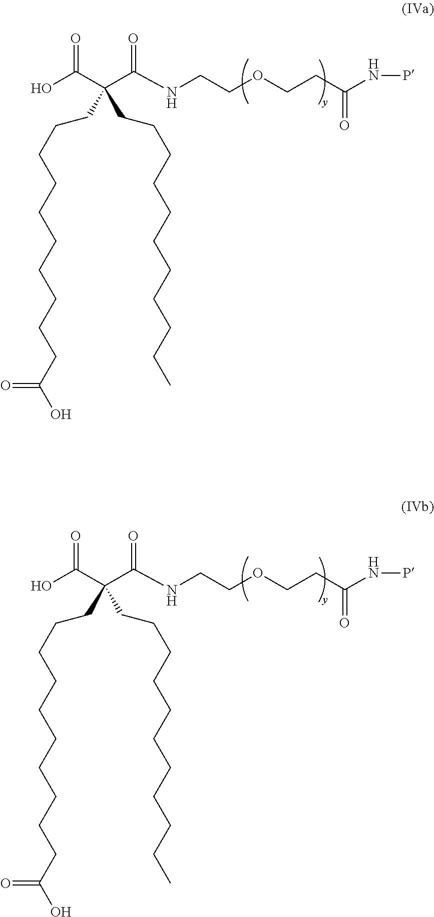

wherein y is an integer selected from 1 to 36.

Another embodiment provides a compound of formula (IVa) or a compound of formula (IVb) as defined herein, wherein y is an integer selected from 2 to 24.

Another embodiment provides a compound of formula (IVa) or a compound of formula (IVb) as defined herein, wherein y is an integer selected from 2, 8, and 24.

Another embodiment provides a compound of formula (IVa) or a compound of formula (IVb) as defined herein, wherein y is 2.

Another embodiment provides a compound of formula (IVa) or a compound of formula (IVb) as defined herein, wherein y is 8.

Another embodiment provides a compound of formula (IVa) or a compound of formula (IVb) as defined herein, wherein y is 24.

Another embodiment provides a compound in accordance to any of the above defined embodiments, or a pharmaceutically acceptable salt thereof, wherein P is selected from

### GLP-1 (7-37): His-Ala-Glu-Gly-Thr-Phe-Thr-Ser-Asp-Val-Ser-Ser-Tyr-Leu-Glu-Gly-Gln-Ala-Ala-Lys-Glu-Phe-Ile-Ala-Trp-Leu-Val-Lys-Gly-Arg-Gly (SEQ ID NO:1), and

a GLP-1 analogue comprising a non-natural amino acid residue in position 7, or in position 8, or in position 7 and 8, relative to the sequence GLP-1 (7-37) (SEQ ID NO:1).

Another embodiment provides a compound in accordance with any of the above defined embodiments, or a pharmaceutically acceptable salt thereof, wherein P is Xaa₇-Xaa₈-Glu-Gly-Thr-Phe-Thr-Ser-Asp-Xaa₆-Ser-Xaa₁₈-Xaa₉-Xaa₂-Glu-Xaa₂₂-Xaa₂₃-Ala-Xaa₂₅-Arg-Xaa₂₇-Phe-Ile-Xaa₃₀-Trp-Leu-Xaa₃₃-Xaa₃₄-Xaa₃₅-Xaa₃₆-Xaa₃₇, (SEQ ID NO:2), (hereinafter P*),

wherein
Xaa₇ is His, imidazopropionyl, α-hydroxy-histidine, D-histidine, desamino-histidine, 2-amino-histidine, β-hydroxy-histidine, homohistidine, N^{α}-acetyl-histidine, N^{α}-formyl-histidine, α-fluoromethyl-histidine, α-methyl-histidine, 3-pyridylalanine, 2-pyridylalanine or 4-pyridylalanine;
Xaa₈ is Ala, Gly, Val, Leu, lie, Thr, Ser, Lys, Aib, (1-aminocyclopropyl) carboxylic acid, (1-aminocyclobutyl) carboxylic acid, (1-aminocyclopentyl) carboxylic acid, (1-aminocyclohexyl) carboxylic acid, (1-aminocycloheptyl) carboxylic acid, or (1-aminocyclooctyl) carboxylic acid;

### Xaa₁₆ is Val or Leu;

### Xaa₁₈ is Ser, Lys or Arg;

### Xaa₁₉ is Tyr or Gln;

### Xaa₂₀ is Leu or Met;

### Xaa₂₂ is Gly, Glu or Aib;

### Xaa₂₃ is Gln, Glu, Lys or Arg;

### Xaa₂₅ is Ala or Val;

### Xaa₂₇ is Glu or Leu;

### Xaa₃₀ is Ala, Glu or Arg;

### Xaa₃₃ is Val or Lys;

### Xaa₃₄ is Lys, Glu, Asn or Arg;

### Xaa₃₅ is Gly or Aib;

Xaa₃₆ is Arg, Gly or Lys, or is absent; and
Xaa₃₇ is Gly, Ala, Glu, Pro, Lys, or is absent.

Another embodiment provides a compound in accordance with the foregoing embodiment, wherein in P*:

Xaa₇ is His or desamino-histidine;

### Xaa₈ is Ala, Gly, Val, Leu, Lys or Aib;

### Xaa₁₆ is Val;

### Xaa₁₈ is Ser;

### Xaa₁₉ is Tyr;

### Xaa₂₀ is Leu;

### Xaa₂₂ is Gly, Glu or Aib;

### Xaa₂₃ is Gln or Glu;

### Xaa₂₅ is Ala;

### Xaa₂₇ is Glu;

### Xaa₃₀ is Ala or Glu;

### Xaa₃₃ is Val;

### Xaa₃₄ is Lys or Arg;

### Xaa₃₅ is Gly or Aib;

Xaa₃₆ is Arg or Lys, or is absent; and
Xaa₃₇ is Gly or is absent.

Another embodiment provides a compound in accordance with the foregoing embodiment, wherein in P*:

### Xaa₇ is His;

### Xaa₈ is Gly or Aib;

### Xaa₁₆ is Val;

### Xaa₁₈ is Ser;

### Xaa₁₉ is Tyr;

### Xaa₂₀ is Leu;

### Xaa₂₂ is Glu or Aib;

### Xaa₂₃ is Gln or Glu;

### Xaa₂₅ is Ala;

### Xaa₂₇ is Glu;

### Xaa₃₀ is Ala;

### Xaa₃₃ is Val;

### Xaa₃₄ is Lys or Arg;

### Xaa₃₅ is Gly or Aib;

### Xaa₃₆ is Arg; and

### Xaa₃₇ is Gly.

Another embodiment provides a compound of formula (I) as defined herein, or a pharmaceutically acceptable salt thereof, wherein P is selected from:

[Aib8, Arg34]GLP-1 (7-37):
(SEQ ID NO: 3)
His-Aib-Glu-Gly-Thr-Phe-Thr-Ser-Asp-Val-Ser-
Ser-Tyr-Leu-Glu-Gly-Gln-Ala-Ala-Lys-Glu-Phe-
Ile-Ala-Trp-Leu-Val-Arg-Gly-Arg-Gly;
and
[Arg34]GLP-1 (7-37):
(SEQ ID NO: 4)
His-Ala-Glu-Gly-Thr-Phe-Thr-Ser-Asp-Val-Ser-
Ser-Tyr-Leu-Glu-Gly-Gln-Ala-Ala-Lys-Glu-Phe-
Ile-Ala-Trp-Leu-Val-Arg-Gly-Arg-Gly.

Another embodiment provides a compound of formula (I) as defined herein, or a pharmaceutically acceptable salt thereof, wherein P is [Aib8, Arg34]GLP-1 (7-37) (SEQ ID NO:3); or alternatively as shown below:

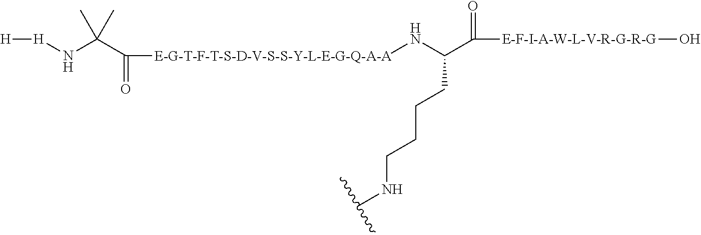

and wherein the wavy line on the amino-acid member Lys indicates the point of attachment to the linker.

Another embodiment provides a compound of formula (I) as defined herein, or a pharmaceutically acceptable salt thereof, which is

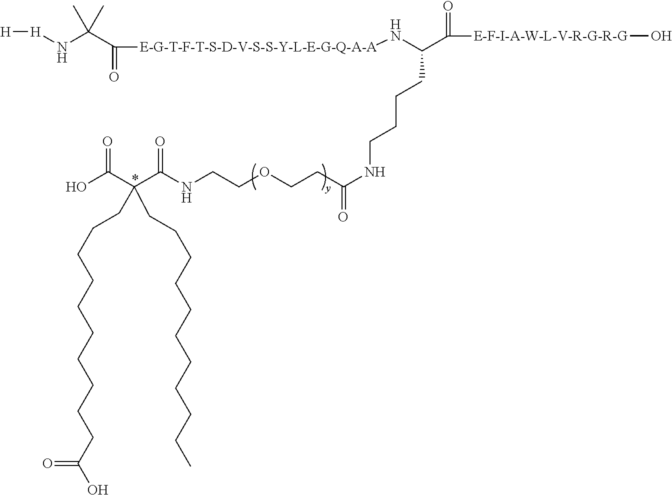

wherein y is an integer selected from 1 to 36, and
wherein the compound is present as a a diastereomeric mixture, a stereochemically enriched mixture or is stereochemically pure in respect of the carbon atom marked *.

Another embodiment provides a compound of formula (I) as defined herein, or a pharmaceutically acceptable salt thereof, which is of formula (X) or of formula (XI):

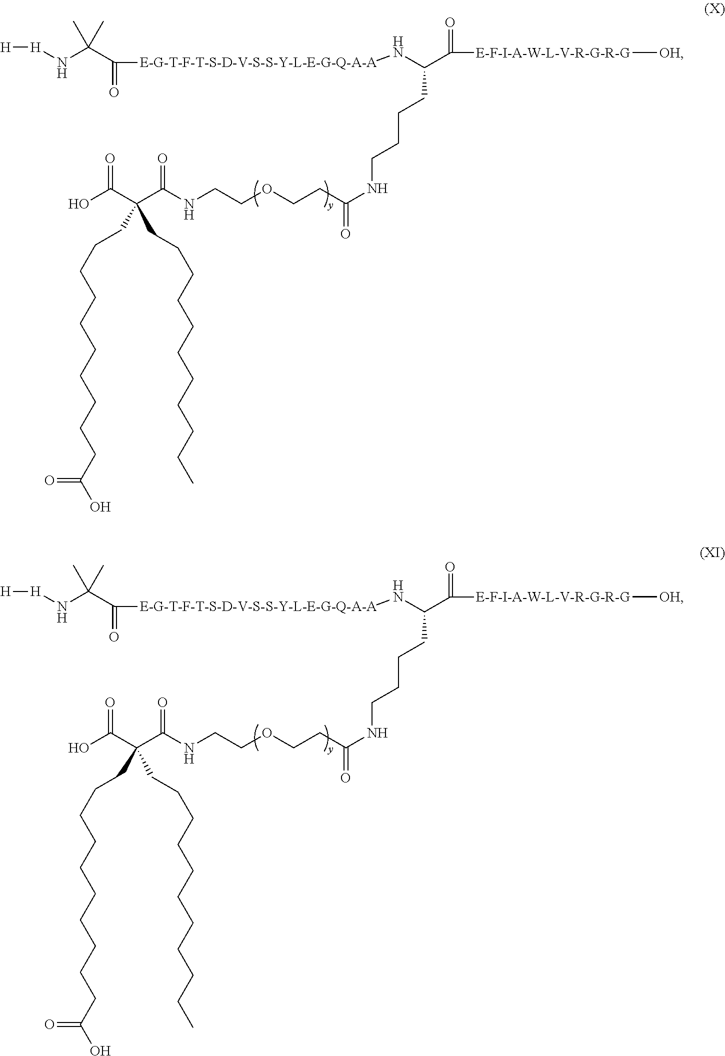

wherein y is an integer selected from 1 to 36.

Another embodiment provides a compound of formula (X) or of formula (XI) as defined herein, wherein y is an integer selected from 2 to 24.

Another embodiment provides a compound of formula (X) or of formula (XI) as defined herein, wherein y is an integer selected from 2, 8 and 24.

Another embodiment provides a compound of formula (X) or of formula (XI) as defined herein, wherein y is 2.

Another embodiment provides a compound of formula (X) or of formula (XI) as defined herein, wherein y is 8.

Another embodiment provides a compound of formula (X) or of formula (XI) as defined herein, wherein y is 24.

Another embodiment provides a compound of formula (I) as defined herein or a pharmaceutically acceptable salt thereof, which is selected from:

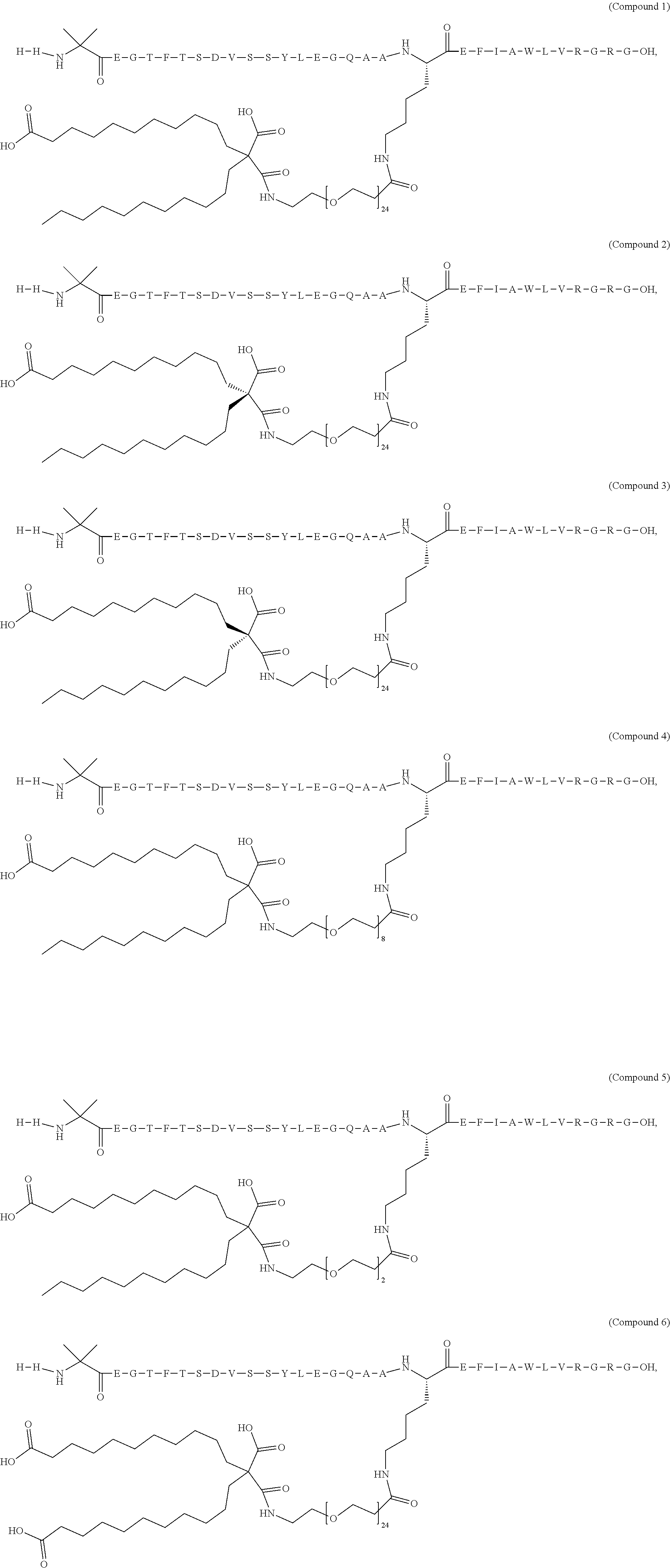

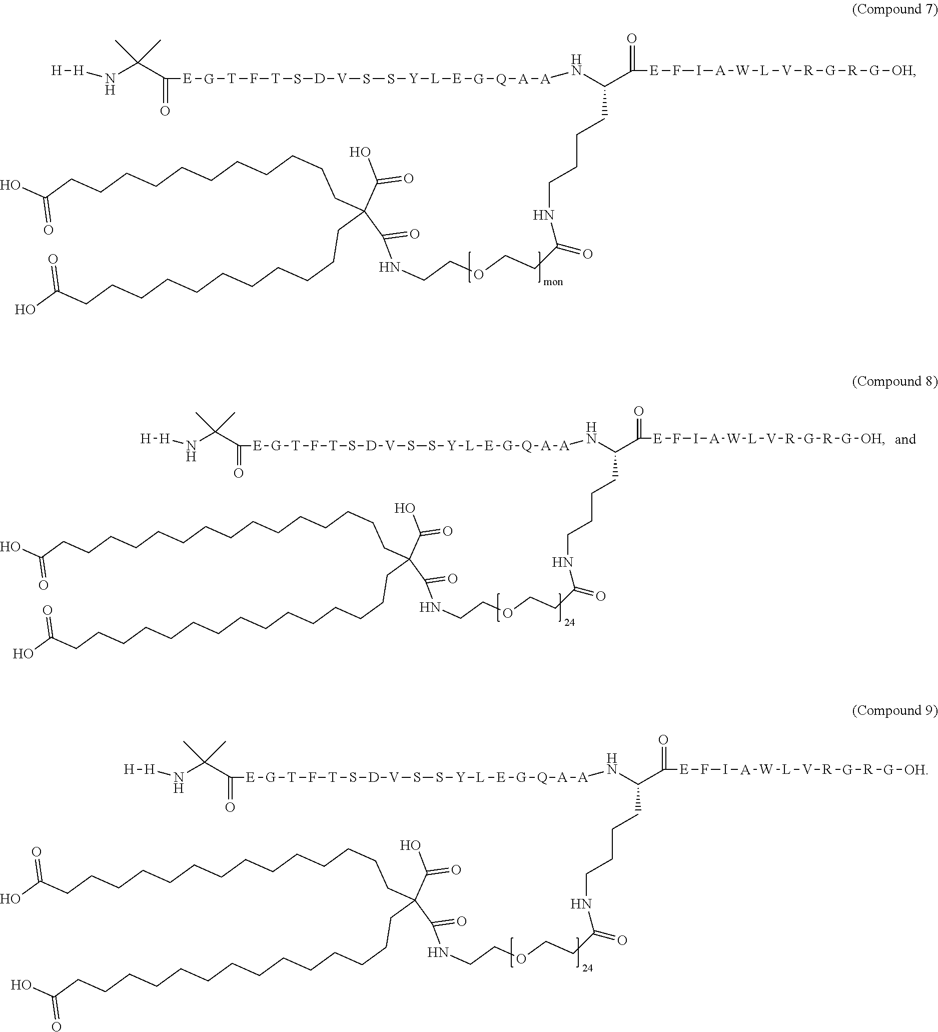

Another embodiment provides a compound of formula (I) as defined herein, or a pharmaceutically acceptable salt thereof, which is:

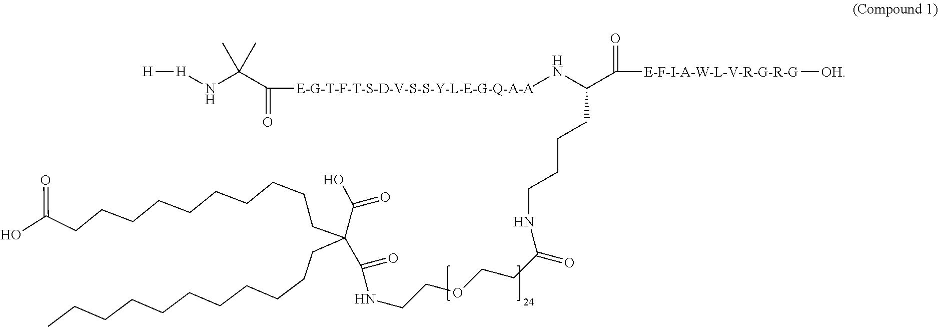

Another embodiment provides a compound of formula (I) as defined herein, or a pharmaceutically acceptable salt thereof, which is:

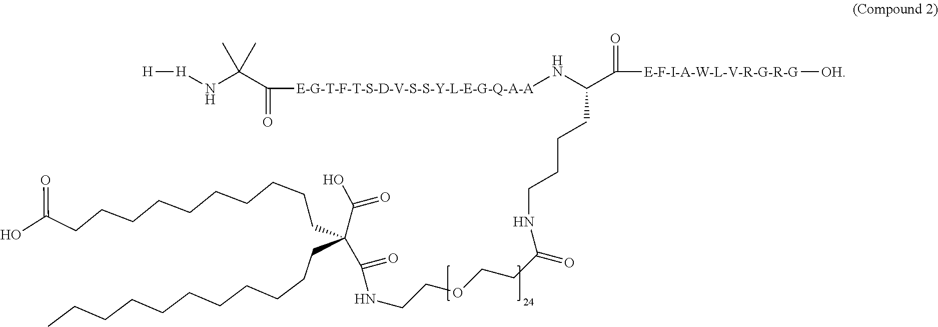

Another embodiment provides a compound of formula (I) as defined herein, or a pharmaceutically acceptable salt thereof, which is:

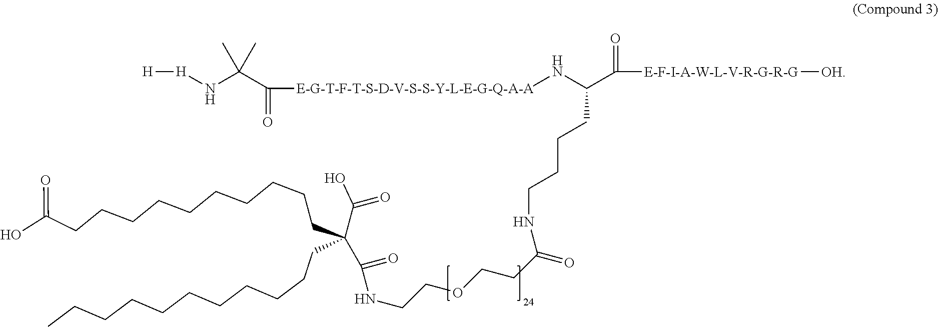

Another embodiment provides a pharmaceutical composition comprising a compound described herein or a pharmaceutically acceptable salt thereof and one or more pharmaceutically acceptable carriers.

Another embodiment provides a pharmaceutical composition in accordance with the previous embodiment wherein the compound is selected from Compound 1, 2, 3, 4, 5, 6, 7, 8, and 9.

Another embodiment provides a pharmaceutical composition in accordance with the previous embodiment wherein the compound is selected from Compound 1, 2, and 3.

Another embodiment provides a combination comprising a therapeutically effective amount of a compound described herein or a pharmaceutically acceptable salt thereof, and one or more therapeutically active agents.

Another embodiment provides a combination in accordance with the previous embodiment wherein the compound is selected from Compound 1, 2, 3, 4, 5, 6, 7, 8, and 9.

Another embodiment provides a combination in accordance with the previous embodiment wherein the compound is selected from Compound 1, 2, and 3.

Another embodiment provides a compound described herein or a pharmaceutically acceptable salt thereof, for use as a medicament.

Another embodiment provides a compound for use in accordance with the previous embodiment, wherein the compound is selected from Compound 1, 2, 3, 4, 5, 6, 7, 8, and 9.

Another embodiment provides a compound for use in accordance with the previous embodiment, wherein the compound is selected from Compound 1, 2, and 3.

Another embodiment provides a compound described herein or a pharmaceutically acceptable salt thereof, for use in the treatment of a disease or disorder selected from obesity, type 2 diabetes mellitus, insulin resistance, hyperinsulinemia, glucose intolerance, hyperglycemia, one or more diabetic complications (including but not limited to chronic kidney disease), diabetic nephropathy, dyslipidemia, metabolic syndrome, progressive liver disease, cardiovascular disease, and neuropathy. As a non-limiting example, the neuropathy is peripheral neuropathy (which may be, e.g., associated with diabetes).

Another embodiment provides a compound as described herein or a pharmaceutically acceptable salt thereof, for use in the treatment of a cardiovascular disease or disorder selected from hypertension, atherosclerosis, peripheral arterial disease, stroke, cardiomyopathy, atrial fibrillation, heart failure (for example, heart failure with reduced ejection fraction (HFrEF), heart failure with mid-range ejection fraction (HFmrEF), and heart failure with preserved ejection fraction (HFpEF), coronary heart disease and arrhythmias (for example, atrial arrhythmias and ventricular arrhythmias).

Another embodiment provides a compound for use in accordance to the previous two embodiments, wherein the compound is selected from Compound 1, 2, 3, 4, 5, 6, 7, 8, and 9.

Another embodiment provides a compound for use in accordance to the previous embodiment, wherein the compound is selected from Compound 1, 2, and 3.

Another embodiment provides a method for treating a patient in need of a therapy being susceptible to an agonist of the Glucagon-like Peptide 1 Receptor (GLP1R), comprising administering to the patient a therapeutically effective amount of a compound as described herein or a pharmaceutically acceptable salt thereof.

Another embodiment provides a method of treatment in accordance to the previous embodiment wherein the compound is selected from Compound 1, 2, 3, 4, 5, 6, 7, 8, and 9.

Another embodiment provides a method of treatment in accordance to the previous embodiment wherein the compound is selected from Compound 1, 2, and 3.

Another embodiment provides a method of treatment in accordance with the previous embodiments, wherein the patient suffers from a disease or disorder selected from obesity, type 2 diabetes mellitus, insulin resistance, hyperinsulinemia, glucose intolerance, hyperglycemia, one or more diabetic complications (including but not limited to chronic kidney disease), diabetic nephropathy, dyslipidemia, metabolic syndrome, progressive liver disease, cardiovascular disease and neuropathy. As a non-limiting example, the neuropathy is peripheral neuropathy (which may be, e.g., associated with diabetes).

Another embodiment provides a method of treatment in accordance to the previous embodiments wherein the patient suffers from a cardiovascular disease or disorder selected from hypertension, atherosclerosis, peripheral arterial disease, stroke, cardiomyopathy, atrial fibrillation, heart failure (for example, heart failure with reduced ejection fraction (HFrEF), heart failure with mid-range ejection fraction (HFmrEF) and heart failure with preserved ejection fraction (HFpEF), coronary heart disease and arrhythmias (for example, atrial arrhythmias and ventricular arrhythmias).

### FURTHER ASPECTS

Depending on the choice of the starting materials and procedures, the compounds can be present in the form of one of the possible stereoisomers or as mixtures thereof, for example as pure optical isomers, or as stereoisomer mixtures, such as racemates and diastereoisomer mixtures, depending on the number of asymmetric carbon atoms. The compounds as described herein are not limited and the compounds include all such possible stereoisomers, including racemic mixtures, diastereomeric mixtures, and optically pure forms. Optically active (R)- and (S)-stereoisomers may be prepared using chiral synthons or chiral reagents, or resolved using conventional techniques. If the compound contains a double bond, the substituent may be E or Z configuration. If the compound contains a disubstituted cycloalkyl, the cycloalkyl substituent may have a cis- or trans-configuration. All tautomeric forms are also intended to be included.

As used herein, the terms “salt” or “salts” refers to an acid addition or base addition salt of a compound of the present disclosure. “Salts” include in particular “pharmaceutical acceptable salts”. The term “pharmaceutically acceptable salts” refers to salts that retain the biological effectiveness and properties of the compounds of this disclosure and, which typically are not biologically or otherwise undesirable. In many cases, the compounds of the present disclosure are capable of forming acid and/or base salts by virtue of the presence of basic nitrogen atoms, for example as found in amino and pyridine groups or other groups similar thereto and/or acidic protons, for example as found in carboxylic acid or 5-oxo-4,5-dihydro-1,2,4-oxadiazol groups, or other groups similar thereto.

Pharmaceutically acceptable acid addition salts can be formed with inorganic acids and organic acids. Inorganic acids from which salts can be derived include, for example, hydrochloric acid, hydrobromic acid, sulfuric acid, nitric acid, phosphoric acid, and the like. Organic acids from which salts can be derived include, for example, acetic acid, propionic acid, glycolic acid, oxalic acid, maleic acid, malonic acid, succinic acid, fumaric acid, tartaric acid, citric acid, benzoic acid, mandelic acid, methanesulfonic acid, ethanesulfonic acid, toluenesulfonic acid, sulfosalicylic acid, and the like.

Pharmaceutically acceptable base addition salts can be formed with inorganic and organic bases. Inorganic bases from which salts can be derived include, for example, ammonium salts and metals from columns I to XII of the periodic table. In certain embodiments, the salts are derived from sodium, potassium, ammonium, calcium, magnesium, iron, silver, zinc, and copper; particularly suitable salts include ammonium, potassium, sodium, calcium, and magnesium salts. Organic bases from which salts can be derived include, for example, primary, secondary, and tertiary amines, substituted amines including naturally occurring substituted amines, cyclic amines, basic ion exchange resins, and the like. Certain organic amines include isopropylamine, benzathine, cholinate, diethanolamine, diethylamine, lysine, meglumine, piperazine, and tromethamine.

In another aspect, compounds of the present disclosure are provided in sodium, potassium, ammonium, calcium, magnesium, iron, silver, zinc, copper, isopropylamine, benzathine, cholinate, diethanolamine, diethylamine, lysine, meglumine, piperazine, or tromethamine salt form.

In another aspect, compounds of the present disclosure are provided in acetate, ascorbate, adipate, aspartate, benzoate, besylate, bromide/hydrobromide, bicarbonate/carbonate, bisulfate/sulfate, camphorsulfonate, caprate, chloride/hydrochloride, chlortheophyllonate, citrate, ethandisulfonate, fumarate, gluceptate, gluconate, glucuronate, glutamate, glutarate, glycolate, hippurate, hydroiodide/iodide, isethionate, lactate, lactobionate, laurylsulfate, malate, maleate, malonate, mandelate, mesylate, methylsulphate, mucate, naphthoate, napsylate, nicotinate, nitrate, octadecanoate, oleate, oxalate, palmitate, pamoate, phosphate/hydrogen phosphate/dihydrogen phosphate, polygalacturonate, propionate, sebacate, stearate, succinate, sulfosalicylate, sulfate, tartrate, tosylate trifenatate, trifluoroacetate, or xinafoate salt form.

Any formula given herein is also intended to represent unlabeled forms as well as isotopically labeled forms of the compounds. Isotopically labeled compounds have structures depicted by the formulae given herein except that one or more atoms are replaced by an atom having a selected atomic mass or mass number. Isotopes that can be incorporated into compounds described herein include, for example, isotopes of hydrogen.

Further, incorporation of certain isotopes, particularly deuterium (i.e., ²H or D) may afford certain therapeutic advantages resulting from greater metabolic stability, for example increased in vivo half-life or reduced dosage requirements or an improvement in therapeutic index or tolerability. It is understood that deuterium in this context is regarded as a substituent of a compound as described herein. The concentration of deuterium, may be defined by the isotopic enrichment factor. The term “isotopic enrichment factor” as used herein means the ratio between the isotopic abundance and the natural abundance of a specified isotope. If a substituent in a compound described herein is denoted as being deuterium, such compound has an isotopic enrichment factor for each designated deuterium atom of at least 3500 (52.5% deuterium incorporation at each designated deuterium atom), at least 4000 (60% deuterium incorporation), at least 4500 (67.5% deuterium incorporation), at least 5000 (75% deuterium incorporation), at least 5500 (82.5% deuterium incorporation), at least 6000 (90% deuterium incorporation), at least 6333.3 (95% deuterium incorporation), at least 6466.7 (97% deuterium incorporation), at least 6600 (99% deuterium incorporation), or at least 6633.3 (99.5% deuterium incorporation). It should be understood that the term “isotopic enrichment factor” can be applied to any isotope in the same manner as described for deuterium.

Other examples of isotopes that can be incorporated into compounds described herein include isotopes of hydrogen, carbon, nitrogen, oxygen, fluorine and sulfur, such as ³H, ¹¹C, ¹³C, ¹⁴C, ¹⁵N, ¹⁸F, ³⁵S respectively. Accordingly, it should be understood that included are any of the compounds described herein that also incorporate one or more of any of the aforementioned isotopes, including for example, radioactive isotopes, such as ³H and ¹⁴C, or those into which non-radioactive isotopes, such as ²H and ¹³C are present. Such isotopically labelled compounds are useful in metabolic studies (with ¹⁴C), reaction kinetic studies (with, for example ²H or ³H), detection or imaging techniques, such as positron emission tomography (PET) or single-photon emission computed tomography (SPECT) including drug or substrate tissue distribution assays, or in radioactive treatment of patients. In particular, an ¹⁸F or labeled compound may be particularly desirable for PET or SPECT studies. Isotopically-labeled compounds described herein can generally be prepared by conventional techniques known to those skilled in the art or by processes analogous to those described in the accompanying Examples and Preparations using an appropriate isotopically-labeled reagent in place of the non-labeled reagent previously employed.

As used herein, the term “pharmaceutical composition” refers to a compound described herein, or a pharmaceutically acceptable salt thereof, together with at least one pharmaceutically acceptable carrier, in a form suitable for oral or parenteral administration.

As used herein, the term “pharmaceutically acceptable carrier” refers to a substance useful in the preparation or use of a pharmaceutical composition and includes, for example, suitable diluents, solvents, dispersion media, surfactants, antioxidants, preservatives, isotonic agents, buffering agents, emulsifiers, absorption delaying agents, salts, drug stabilizers, binders, excipients, disintegration agents, lubricants, wetting agents, sweetening agents, flavoring agents, dyes, and combinations thereof, as would be known to those skilled in the art (see, for example, Remington The Science and Practice of Pharmacy, 22^{nd} Ed. Pharmaceutical Press, 2013, pp. 1049-1070).

The term “a therapeutically effective amount” of a compound described herein refers to an amount of that compound that will elicit the biological or medical response of a subject. As a non-limiting set of examples, such a therapeutically effective amount of a compound described herein could, for example, agonize GLP1R activity, ameliorate one or more symptoms, alleviate one or more conditions, slow or delay the progression of a disease, disorder or condition, or prevent a disease, disorder or condition.

As used herein, the term “a therapeutically effective amount” refers to the amount of a compound described herein that, when administered to a subject, at least partially alleviates, prevents and/or ameliorates a condition, or a disorder or a disease responsive to increasing or agonizing the activity of GLP1R. In another embodiment, the term “a therapeutically effective amount” refers to the amount of a compound described herein that, when administered to a subject, a cell, or a tissue; or a non-cellular biological material; or a medium, at least partially increases or agonizes the activity of GLP1R; or at least partially increases or agonizes the expression of GLP1R. In another embodiment, the term “a therapeutically effective amount” refers to the amount of a compound described herein that, when administered to a subject, causes an observable level of one or more desired biological or medicinal responses, for example selected from: lowering glucose levels (such as lowering blood glucose levels), increasing insulin sensitivity, improving glucose homeostasis, lowering triglyceride or cholesterol levels, reducing body weight, reducing food intake and reducing body fat mass (such as peripheral fat and/or visceral fat).

As used herein, the term “patient” or “subject” is interchangeable and refers to primates (e.g., humans, male or female; or non-human primates), dogs, rabbits, guinea pigs, pigs, rats and mice. In certain embodiments, the subject is a primate. In yet other embodiments, the subject is a human.

As used herein, the terms “agonize”, “agonism”, and “agonizing” refer to an increase of signaling of GLP1R, for example as measured by an increase in intracellular cyclic adenosine mono-phosphate (cAMP).

As used herein, the term “treat”, “treating”, or “treatment” of any disease, disorder or condition refers to alleviating or ameliorating the disease, disorder or condition (i.e., slowing or arresting the development or progression of the disease, disorder or condition, or at least one of the clinical symptoms thereof); or alleviating or ameliorating at least one physical parameter or biomarker associated with the disease, disorder or condition, including those which may not be discernible to the patient.

As used herein, the term “prevent”, “preventing”, or “prevention” of any disease, disorder or condition refers to the prophylactic treatment of the disease, disorder or condition; or delaying the onset or progression of the disease, disorder or condition.

As used herein, a subject is “in need of” a treatment if such subject would benefit biologically, medically or in quality of life from such treatment.

As used herein, the term “a”, “an”, “the”, and similar terms used (especially in the context of the claims) are to be construed to cover both the singular and plural unless otherwise indicated herein or clearly contradicted by the context.

All methods described herein can be performed in any suitable order unless otherwise indicated herein or otherwise clearly contradicted by context. The use of any and all examples, or exemplary language (e.g., “such as”) provided herein is intended merely to better illuminate the compositions and methods or uses provided herein and does not pose a limitation on the scope otherwise claimed.

Any asymmetric atom (e.g., carbon or the like) of the compound(s) described herein can be present in racemic or enantiomerically enriched, for example the (R)-, (S)- or (R,S)-configuration. In certain embodiments, each asymmetric atom has at least 50% enantiomeric excess, at least 60% enantiomeric excess, at least 70% enantiomeric excess, at least 80% enantiomeric excess, at least 90% enantiomeric excess, at least 95% enantiomeric excess, or at least 99% enantiomeric excess in the (R)- or (S)-configuration.

Accordingly, as used herein a compound as described herein may be in the form of one of the possible stereoisomers, rotamers, atropisomers, tautomers or mixtures thereof, for example, as substantially pure diastereomers, optical isomers (antipodes), racemates or mixtures thereof.

Any resulting mixtures of stereoisomers can be separated on the basis of the physicochemical differences of the constituents, into the pure or substantially pure geometric or optical isomers, diastereomers, racemates, for example, by chromatography and/or fractional crystallization.

Any resulting racemates of compounds described herein or of intermediates can be resolved into the optical antipodes by known methods, e.g., by separation of the diastereomeric salts thereof, obtained with an optically active acid or base, and liberating the optically active acidic or basic compound. In particular, a basic moiety may thus be employed to resolve the compounds described herein into their optical antipodes, e.g., by fractional crystallization of a salt formed with an optically active acid, e.g., tartaric acid, dibenzoyl tartaric acid, diacetyl tartaric acid, di-O,O′p-toluoyl tartaric acid, mandelic acid, malic acid or camphor-10-sulfonic acid. Racemic compounds described herein or racemic intermediates can also be resolved by chiral chromatography, e.g., high pressure liquid chromatography (HPLC) using a chiral adsorbent.

The compounds of the present application can be prepared by those skilled in the art of organic synthesis using commercially available starting materials, compounds known in the literature, or from readily prepared intermediates, by employing standard synthetic methods and procedures either known to those skilled in the art, or which will be apparent to the skilled chemist in light of the teachings herein.

The compounds described herein may be prepared by methods known in the art of organic synthesis as set forth in part by the following synthetic schemes. In the schemes described below, it is well understood that protecting groups for sensitive or reactive groups are employed where necessary in accordance with general principles of chemistry. Protecting groups are manipulated according to standard methods of organic synthesis as described for example in Protective Groups in Organic Synthesis, 3rd edition, John Wiley & Sons: New York, 1999 or Protecting Groups, 3rd edition, Thieme, Stuttgart, 2004. Protective groups are removed at a convenient stage of the compound synthesis using methods that are readily apparent to those skilled in the art.

Those skilled in the art will recognize if a stereocenter exists in the compounds disclosed herein. Resolution of the final product, an intermediate, or a starting material may be affected by any suitable method known in the art. See, for example, “Stereochemistry of Organic Compounds” by E. L. Eliel, S. H. Wilen, and L. N. Mander (Wiley-Interscience, 1994).

The compounds described herein may be made from commercially available starting materials or synthesized using known organic, inorganic, and/or enzymatic processes.

### Preparation of Compounds

The compounds described herein may be prepared in a number of ways well known to those skilled in the art of organic synthesis. By way of example, compounds of the present disclosure may be synthesized using the methods described below, together with synthetic methods known in the art of synthetic organic chemistry, or variations thereon as appreciated by those skilled in the art.

### General Synthetic Procedure

Compounds described herein may be manufactured as shown in detail in the experimental section (CHEMISTRY SECTION), for example:

### General Scheme (1): Synthesis of the Fatty Acid Moiety, Compound of Formula (i):

The general way of preparing compounds of formula (i) is outlined in General Scheme (I).

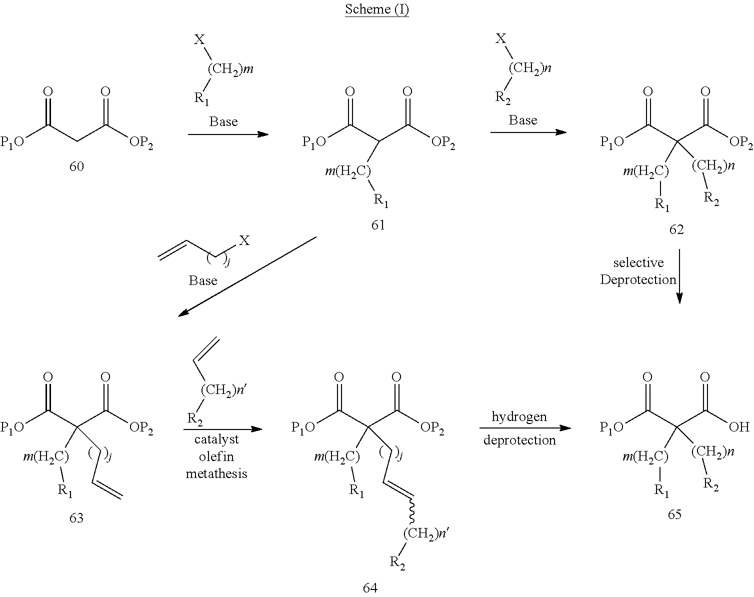

A malonic acid derivative (60) may be reacted with R₁—(CH₂)ₘ—X in the presence of a base, e.g. sodium hydride, potassium or cesium carbonates, sodium hydroxide, lithium diisopropyl amide, sodium bis(trimethylsilyl)amide, and the like, and in the presence or absence of a solvent such as DMF, THF or dimethyl acetamide and at around RT or above or below, yielding the alkylated intermediate (61), which is then reacted with R₂—(CH₂)ₙ—X in the presence of a base to provide the di-alkylated intermediate (62). The variables R₁, R₂, n and m have the meanings as defined herein, X is a leaving group selected from halogen (e.g. Br, Cl, I), trifluoromethanesulfonyloxy and the like, and P₁ and P₂ are carboxylic acid protective group such as for example methyl, ethyl, tert-butyl, methoxybenzyl, benzyl, trimethylsilyl, t-butyldimethylsilyl or 2-alkyl 1,3 oxazolines.

Depending on the protecting groups P₁ and P₂, intermediate (62) is then either reacted with a base, e.g. NaOH, KOH, or LiOH, or with an acid selected from, but not limited to, TFA, HCl, or BCl₃, or in case the protecting groups P₁ and P₂ are benzyl or methoxybenzyl, intermediate (62) is typically reacted with hydrogen in the presence of a catalyst such as, but not limited to, palladium-on-carbon, to provide compound (65), which corresponds to a compound of formula (i), i.e. when P, is hydrogen.

Alternatively, intermediate (61) may be reacted with CH₂═CH—(CH₂)ⱼ—X, wherein j is 1-10 and X is as defined herein, e.g. allyl bromide, in the presence of a base such as NaH, potassium or cesium carbonates, sodium hydroxide, lithium diisopropyl amide and the like, and in the presence or absence of a solvent such as DMF, THF or dimethyl acetamide to yield the unsaturated di-alkylated intermediate 63, which may be separated into its R or S enantiomer by chromatography. Intermediate 63 is then reacted in the presence of an excess, e.g. 2 equivalents of an alkylating reagent R₂—(CH₂)_{n′}—CH═CH₂, wherein n′ is 5-27, and an olefin metathesis catalyst, e.g. Grubbs II in the presence of a solvent such as DCM or THF to yield intermediate 64, which may be reacted with hydrogen in the presence of a catalyst, e.g. Pd/C in the presence of a solvent, e.g. THF, methanol or the like and optionally followed by a deprotection reaction, e.g. provided P₂ is not a benzyl-group, typically as disclosed in the reaction of intermediate 62 into intermediate 65; e.g. with NaOH, KOH, or LiOH, in methanol, ethanol or dioxane or with an acid selected from, but not limited to, TFA, HCl, or BCl₃. The double bond in the side chain may also be hydrogenated after the linker is attached to the fatty acid as shown in scheme (II). The parameters j and n′ together with a CH═CH group are chosen to provide a chain length determined by n in intermediate 65, i.e. (CH₂)ₙ.

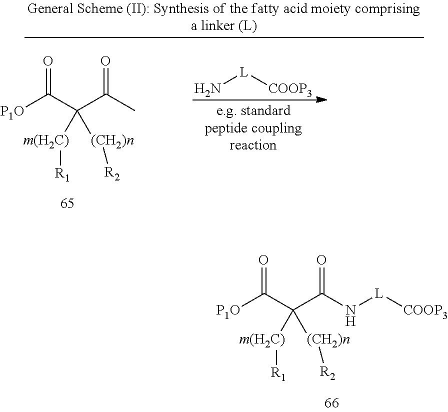

The general way of preparing intermediate 66 by using intermediate 65 is outlined in General Scheme (II). The fatty acid derivative 65 may be typically reacted with an amino acid derivative of formula H₂N-L-COOP₃, wherein P₃ is hydrogen or a carboxylic acid protective group (e.g., methyl, ethyl, tert-butyl, methoxybenzyl, benzyl, trimethylsilyl, t-butyldimethylsilyl or 2-alkyl 1,3 oxazolines) and L is a linker as described herein, with the proviso that the linker L in the amino acid derivative of formula H₂N-L-000P₃ is shown together with its terminal groups, i.e. NH₂ and COOP₃, in the presence of a coupling reagent, e.g. carbonyldiimidazole (DCC) in the presence or absence of a base, e.g. N,N-diisoproplyl ethylamine or K₂CO₃, and in the presence or absence of a solvent, e.g. DMF, to obtain the derivatized fatty acid derivative (66).

Standard peptide coupling reactions include, for example, conversion of the carboxylic acid group into an activated form thereof, e.g., to a corresponding pyrrolidine-2,5-dione group, e.g. by using standard N-hydrosuccinimide chemistry, or by reacting a carbonic acid group with reagents such as triphosgene, carbonyldiimidazole, 4-nitrophenyl chloroformate, or disuccinimidyl carbonate, to a corresponding carbonic acid halide, by using reagents such as thionyl chloride or oxalyl chloride, or by converting a carbonic acid group to a corresponding mixed anhydride using reagents such as ClC(O)O-isobutyl, 2,4,6-trichlorobenzoyl chloride or propyl phosphonic acid anhydride cyclic trimer (T3P), followed by reaction of the oxazolidine-2,5-dione, the acid halide, or the mixed anhydride in the presence or the absence of a base such as a tertiary amine (e.g. triethylamine or N,N-diisoproplyl ethylamine) or an inorganic base e.g. K₂CO₃. Alternatively, peptide coupling reactions reagents include dicyclohexylcarbodiimide (DCC), diisopropylcarbodiimide (DIC), 1-ethyl-3-(3-dimethyllaminopropyl)carbodiimide hydrochloride (EDC HCl), benzotriazole-1-yl-oxy-tris-pyrrolidino-phosphonium hexafluorophosphate (PyBOP), or benzotriazole-1-yl-oxy-tris-(dimethylamino)-phosphonium hexafluorophosphate (BOP) in presence of or absence of a reagent such as 1-hydroxybenzotriazole, 1-hydroxy-7-azabenzotriazole, or dimethylaminopyridine.

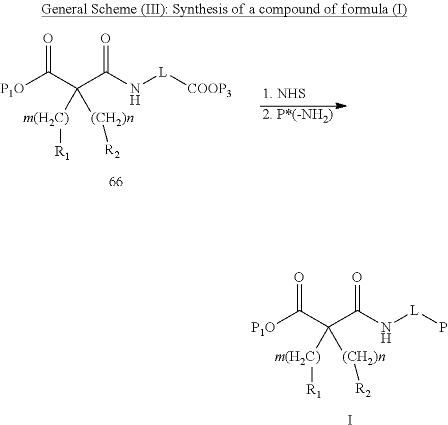

The general way of preparing compounds of formula (I) by using intermediate 66 is outlined in General Scheme (III). Provided P₃ is a protecting group (e.g. not hydrogen), the fatty acid derivative of formula (66) is converted to its carboxylic acid, e.g. using an acid, e.g., HCl or p-toluenesulfonic acid, in the presence or absence of a solvent, e.g., methanol, and is then converted into an activated carbonic acid ester, e.g. an NHS-ester, e.g. using DCC and N-hydroxysuccinimide (NHS) in the presence or absence of a solvent, e.g., DCM or THF, which is then reacted with a GLP-1 or GLP-1 analogue P* having a free —NH₂ group, e.g., in the presence of piperidine and a solvent, e.g., DMF or DMA; wherein the variables P* and P have the meanings as defined herein (as shown in General Scheme (III)).

A mixture of enantiomers, diastereomers, and cis/trans isomers resulting from the process described above can be separated into their single components by chiral salt technique, chromatography using normal phase, reverse phase or chiral column, depending on the nature of the separation.

Any resulting racemates of compounds of the present disclosure or of intermediates can be resolved into the optical antipodes by known methods, e.g., by separation of the diastereomeric salts thereof, obtained with an optically active acid or base, and liberating the optically active acidic or basic compound. In particular, a basic moiety may thus be employed to resolve the compounds of the present disclosure into their optical antipodes, e.g., by fractional crystallization of a salt formed with an optically active acid, e.g., tartaric acid, dibenzoyl tartaric acid, diacetyl tartaric acid, di-O,O′-p-toluoyl tartaric acid, mandelic acid, malic acid, or camphor-10-sulfonic acid. Racemic compounds of the present disclosure or racemic intermediates can also be resolved by chiral chromatography, e.g., high pressure liquid chromatography (HPLC) using a chiral adsorbent.

### Pharmaceutical Compositions

The pharmaceutical composition described herein comprises a compound as described herein, or a pharmaceutically acceptable salt thereof, and a pharmaceutically acceptable carrier. In a further embodiment, the composition comprises at least two pharmaceutically acceptable carriers, such as those described herein. A pharmaceutical composition may be formulated for particular routes of administration such as oral administration, parenteral administration (e.g., by injection, infusion, transdermal, or topical administration), and rectal administration. Topical administration may also pertain to inhalation or intranasal application. The pharmaceutical compositions described herein may be made up in a solid form (including, without limitation, capsules, tablets, pills, granules, powders, or suppositories), or in a liquid form (including, without limitation, solutions, suspensions or emulsions). Tablets may be either film coated or enteric coated according to methods known in the art. Typically, the pharmaceutical compositions are tablets or gelatin capsules comprising the active ingredient together with one or more of:
a) diluents, e.g., lactose, dextrose, sucrose, mannitol, sorbitol, cellulose and/or glycine;
b) lubricants, e.g., silica, talcum, stearic acid, its magnesium or calcium salt and/or polyethyleneglycol;
   for tablets also
c) binders, e.g., magnesium aluminum silicate, starch paste, gelatin, tragacanth, methylcellulose, sodium carboxymethylcellulose and/or polyvinylpyrrolidone; if desired
d) disintegrants, e.g., starches, agar, alginic acid or its sodium salt, or effervescent mixtures; and
e) absorbents, colorants, flavors and sweeteners.

Pharmaceutical compositions suitable for injectable use typically include sterile aqueous solutions (where water soluble) or dispersions and sterile powders for the extemporaneous preparation of sterile injectable solutions or dispersion.

For intravenous administration, suitable carriers include physiological saline, bacteriostatic water, Cremophor EL™ (BASF, Parsippany, N.J.) or phosphate buffered saline (PBS). In all cases, the composition should be sterile and should be fluid to the extent that easy syringability exists. Preferred pharmaceutical formulations are stable under the conditions of manufacture and storage and must be preserved against the contaminating action of microorganisms such as bacteria and fungi. In general, the relevant carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, amino acids, sorbitol, sodium chloride in the composition. Prolonged absorption of the injectable compositions can be brought about by including in the composition an agent which delays absorption, for example, aluminum monostearate and gelatin. In some embodiments, a multifunctional excipient such as recombinant albumin may be incorporated into the formulation process to facilitate the stabilization of the instant compounds from degradation or aggregation, to improve solubility and assist in the administration and release of the active component. (BioPharm International, 2012, Vol 23, Issue 3, pp 40-44).

Certain injectable compositions are aqueous isotonic solutions or suspensions, and suppositories are advantageously prepared from fatty emulsions or suspensions. Said compositions may be sterilized and/or contain adjuvants, such as preserving, stabilizing, wetting or emulsifying agents, solution promoters, salts for regulating the osmotic pressure and/or buffers. In addition, they may also contain other therapeutically valuable substances. Said compositions are prepared according to conventional mixing, granulating or coating methods, respectively, and contain 0.1-75%, or contain 1-50%, of the active ingredient.

Sterile injectable solutions can be prepared by incorporating the active compound in the required amount in an appropriate solvent with one or a combination of ingredients enumerated above, as required, followed by filtration sterilization. Generally, dispersions are prepared by incorporating the active compound into a sterile vehicle which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

The compounds described herein whether in free form or in pharmaceutically acceptable salt form exhibit valuable pharmacological properties, for example, as agonists of GLP1R, e.g., as indicated in in vitro and in vivo tests provided herein, and are therefore indicated for therapy or for use as research chemicals, e.g., as tool compounds.

### Utility Lists

Compounds described herein may be useful in the treatment of metabolic and related diseases, disorders and conditions, e.g., selected from:

Obesity, type 2 diabetes mellitus, insulin resistance, hyperinsulinemia, glucose intolerance, hyperglycemia, one or more diabetic complications (including but not limited to chronic kidney disease), diabetic nephropathy, dyslipidemia, metabolic syndrome, progressive liver disease, cardiovascular disease and neuropathy. As a non-limiting example, the neuropathy is peripheral neuropathy (which may be, e.g., associated with diabetes).

The progressive liver disease may be, for example, non-alcoholic fatty liver disease (FLD or NAFLD), and for example non-alcoholic steatohepatitis (NASH).

The cardiovascular disease may be selected from: Hypertension, atherosclerosis, peripheral arterial disease, stroke, cardiomyopathy, atrial fibrillation, heart failure (for example heart failure with reduced ejection fraction (HFrEF), heart failure with mid-range ejection fraction (HFmrEF) and heart failure with preserved ejection fraction (HFpEF), coronary heart disease and arrhythmias (for example atrial arrhythmias and ventricular arrhythmias).

The compounds of the invention may be useful in the treatment of several diseases, disorders or conditions co-occurring in a subject (termed ‘co-morbidities’). Co-morbidities, for example, may be those in subjects which are type 2 diabetic and are additionally obese and/or additionally exhibit heart failure and/or NASH. For example an obese subject may also exhibit type 2 diabetes and/or exhibit cardiovascular disease (for example heart failure). Such subject may also exhibit a progressive liver disease (for example NASH). For example, an obese subject may also exhibit type 2 diabetes and/or exhibit cardiovascular disease (for example heart failure) and/or exhibit a progressive liver disease (for example NASH). The subject may also have high blood pressure and/or high blood cholesterol level. The subject may also suffer from peripheral neuropathy.

As used herein, the indications disclosed in the above utility sections may be referred to hereinafter as the “afore-mentioned lists”.

In an embodiment, the disease, disorder or condition is selected from obesity, type 2 diabetes, atherosclerosis, heart failure (in particular heart failure with preserved ejection fraction) and NASH.

In an embodiment, the disease, disorder or condition is selected from obesity, type 2 diabetes, atherosclerosis and heart failure (in particular heart failure with preserved ejection fraction).

Another aspect of the disclosure relates to a method of treating, preventing, inhibiting, or eliminating a disease or disorder in a patient associated with modulation of GLP1R. The method comprises administering to a patient in need of a treatment for diseases or disorders associated with modulation of GLP1R an effective amount of a compound as described herein or a pharmaceutically acceptable salt thereof or a pharmaceutical composition comprising a compound described herein or a pharmaceutically acceptable salt thereof and one or more pharmaceutically acceptable carriers.

Thus, as a further aspect, the provided herein is the use of a compound described herein or a pharmaceutically acceptable salt thereof, in therapy. In a further embodiment, the therapy is treatment of a disease, disorder or condition which may be treated by agonism of GLP1R. In another embodiment, the therapy is treatment of a disease, disorder or condition selected from any of the afore-mentioned lists.

Thus, as a further aspect, provided herein is a compound described herein or a pharmaceutically acceptable salt thereof for use in therapy. In a further embodiment, the therapy is treatment of a disease, disorder or condition which may be treated by agonism of GLP1R. In another embodiment, the therapy is treatment of a disease, disorder or condition selected from any of the afore-mentioned lists.

In another aspect, provided herein is a method of treating a disease, disorder or condition in a patient, which is treatable by agonism of GLP1R, comprising administration of a therapeutically effective amount of a compound described herein or a pharmaceutically acceptable salt thereof.

In another embodiment, provided herein is a method of treating a disease, disorder or condition in a subject in need thereof, the method comprising administering to the subject a therapeutically effective amount of a compound described herein wherein the disease, disorder or condition is selected from any of the afore-mentioned lists.

In a further aspect, provided herein is the use of a compound described herein or a pharmaceutically acceptable salt thereof, for the manufacture of a medicament. In a further embodiment, the medicament is for treatment of a disease which may be treated by agonism of GLP1R. In another embodiment, the disease is selected from any of the afore-mentioned lists.

Moreover, the invention provides the use of any compound or a pharmaceutically acceptable salt thereof described herein for treating a disease, disorder, or condition selected from any of the afore-mentioned lists.

The term “metabolic disorders” or “metabolic diseases” refers to an associated cluster of traits that includes, but is not limited to, obesity, glucose intolerance, insulin resistance, hyperinsulinemia, excess visceral adiposity, hypertension, dyslipidemia characterized by high triglycerides, low high-density lipoprotein (HDL)-cholesterol, and high low-density lipoprotein (LDL) cholesterol. Subjects having metabolic disease or disorder are at risk of developing of type 2 diabetes mellitus and, for example, atherosclerosis.

The term “obesity” in human adults refers to a Body Mass Index (BMI) of 30 or greater (Centers for Disease Control and Prevention). Such subject may also be referred to as obese. This is referred to as Class I obesity. Class II obesity includes individuals with a BMI of 35-39.9 and Class Ill obesity refers to individuals with a BMI of greater than 40. Body mass index (BMI) is a measure of body fat based on height and weight. The formula for calculation is BMI=weight in kilograms/height in meters². In an embodiment the human subject suffering from obesity has a BMI of ≥30 or ≥35 or a BMI in the range ≥35 to <40 or ≥30 to <40. The amount <40 can, for example, be 39.9. In some embodiments the obesity is severe obesity or morbid obesity, wherein the human subject has a BMI of ≥40.

The term “type 2 diabetes mellitus” is a condition characterized by persistently high glucose levels both in the fasted and fed state which results from a combination of impaired glucose utilization and excess glucose production. This may result from either inadequate production of insulin from the pancreas or peripheral insulin resistance.

The term “insulin resistance” as used herein refers to a condition where a normal quantity of insulin cannot induce the expected physiological response and cannot activate downstream pathways. In many examples insulin beyond the physiologic range either endogenously produced or exogenously administered, is sufficient to induce a complete or partial biologic response to induce the expected physiological response.

The term “hyperinsulinemia” refers to a condition where excess insulin may be detected in the blood.

The term “glucose intolerance” encompasses any disorder characterized by a clinical symptom or a combination of clinical symptoms that is associated with an elevated level of basal or post-prandial glucose and/or an elevated level of insulin or abnormal glucose stimulated insulin release or HOMA-IR (homeostatic model assessment of insulin resistance) in a subject relative to a healthy individual. Elevated levels of glucose and/or insulin may be manifested in the following diseases, disorders and conditions: obesity, metabolic syndrome, impaired glucose tolerance, type II diabetes, gestational diabetes, type I diabetes, insulin resistance, hyperinsulinemia, lipodystrophy, lipoatrophy and various MODY (maturity onset diabetes of the young) mutations. The GLP1R agonists of the present disclosure, and compositions thereof, can be used, for example, to achieve and/or maintain glucose homeostasis, e.g. to reduce glucose level in the bloodstream and/or to reduce insulin level to a range found in a healthy subject.

The term “hyperglycemia”, as used herein, refers to a condition in which an elevated amount of glucose circulates in the blood plasma of a subject relative to a healthy individual. Hyperglycemia can be diagnosed using methods known in the art, including measurement of fasting blood glucose levels as described herein.

The term “diabetic complications” are problems caused by persistently high blood glucose levels that damage other organs including kidneys, peripheral limbs, and eyes (e.g. retinopathies) or induce vascular disease and neuropathy. Impaired vascular function contributes to erectile dysfunction and can lead to increased risk of skin infections. Diabetes also increases the risk for heart disease and bone and joint disorders. Other long-term complications of diabetes include excess risk of cancer including hepatocellular carcinoma, endometrial cancer, breast cancer, and pancreatic cancer.

The term “diabetic nephropathy” is a condition resulting from diabetes and caused by damage to blood vessels and other cells in the kidney that reduces kidney function.

The term “dyslipidemia” refers to complex disorders of lipoprotein metabolism, including lipoprotein overproduction or abnormal metabolism. Dyslipidemias may be manifested by elevation of the total cholesterol, low-density lipoprotein (LDL) cholesterol and triglyceride concentrations, and a decrease in high-density lipoprotein (HDL) cholesterol concentration in the blood.

The term “metabolic syndrome” refers to a cluster of risk factors that raises the risk for cardiovascular disease including coronary artery disease, heart failure with reduced ejection fraction, heart failure with preserved ejection fraction, cerebrovascular disease and peripheral vascular disease. These risk factors include: abdominal fat, high blood sugar (at least 110 milligrams per deciliter (mg/dl)) after fasting; high triglycerides (at least 150 mg/dL) in the bloodstream; low HDL (less than 40 mg/dl); and, blood pressure of 130/85 mmHg or higher (World Health Organization).

The term “progressive liver disease” refers to the progression from a benign state of hepatosteatosis evidenced by fibrosis and cirrhosis, which predispose to hepatocellular carcinoma. The progression of obesity associated non-alcoholic fatty liver (NAFL) to NASH, fibrosis and cirrhosis is well documented.

The term “non-alcoholic fatty liver disease (FLD)”, also known as NAFLD is a condition wherein excess lipid accumulates in hepatocytes, which can result from either excess de novo lipogenesis in the liver or abnormal clearance and oxidation of fatty acids. NAFLD is excluded from other causes of liver disease including alcoholic liver disease and viral liver disease. NAFLD includes three histologic entities that reflect progression of the disease: fatty liver, hepatosteatosis and fibrosis or cirrhosis. The most common cause of NAFLD is obesity, although NAFLD can also be seen in lean individuals. Accumulation of fat may progress inflammation accompanied by infiltration of macrophages and changes in hepatocyte histology including ballooning, termed steatohepatitis and referred to as non-alcoholic steatohepatitis (NASH). NASH may progress to fibrosis with interlobular bridging fibrosis or cirrhosis. As used herein, the term NASH may encompass steatohepatitis, hepatocellular ballooning and lobular inflammation.

The term “cardiovascular diseases” are diseases related to the heart or blood vessels.

The term “atherosclerosis” refers to vascular disease characterized by irregularly distributed lipid deposits in the intima of large and medium-sized arteries, sometimes causing narrowing of arterial lumens and proceeding eventually to fibrosis and calcification. Lesions are usually focal and progress slowly and intermittently. Limitation of blood flow accounts for most clinical manifestations, which vary with the distribution and severity of lesions.

The term “peripheral arterial disease” refers to when a build-up of fatty deposits in the arteries restricts blood supply to leg muscles.

The term “stroke” refers to when the blood supply to part of the brain is cut off.

The term “cardiomyopathy” is defined as acquired or congenital structural abnormalities of the atrial or ventricular myocardium that may affect cardiac function, or physiology, and conduction.

The term “heart failure” refers to when the heart has reduced ability to pump blood and can include heart failure with preserved ejection fraction (HFpEF), heart failure with reduced ejection fraction (HFrEF) and heart failure with mid-range ejection fraction (HFmrEF).

The term “coronary heart disease”, also called coronary artery disease, is a narrowing of the arteries that supply blood to the heart.

The term “arrhythmias” refers to abnormal heart rhythm and can include atrial arrhythmias, atrial fibrillation and ventricular arrhythmias.

The term “neuropathy” refers to when nerves are damaged. The term includes peripheral neuropathy which develops when nerves in the extremities such as hands, feet and arms are damaged. Diabetes is a common cause of peripheral neuropathy.

Another embodiment provides a compound of formula (I) as defined herein or a pharmaceutically acceptable salt thereof, which is:

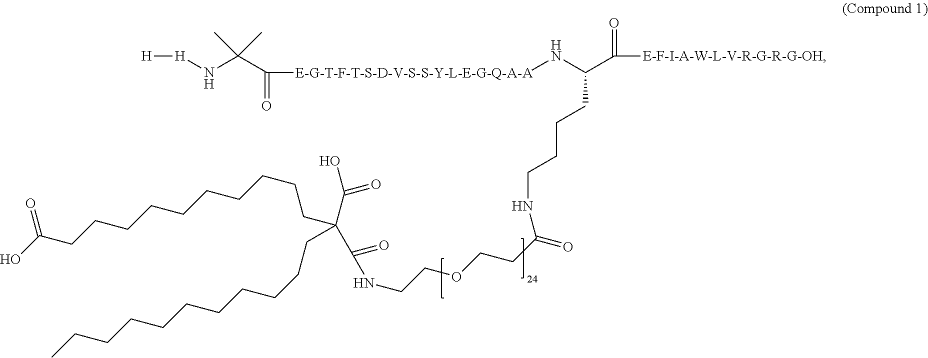

for use in the treatment of a disease or disorder selected from obesity, type 2 diabetes mellitus, insulin resistance, hyperinsulinemia, glucose intolerance, hyperglycemia, diabetic complications (including but not limited to chronic kidney disease), diabetic nephropathy, dyslipidemia, metabolic syndrome, progressive liver disease, cardiovascular disease and neuropathy (in particular peripheral neuropathy, e.g. associated with diabetes.

Another embodiment provides a compound of formula (I) as defined herein or a pharmaceutically acceptable salt thereof, which is:

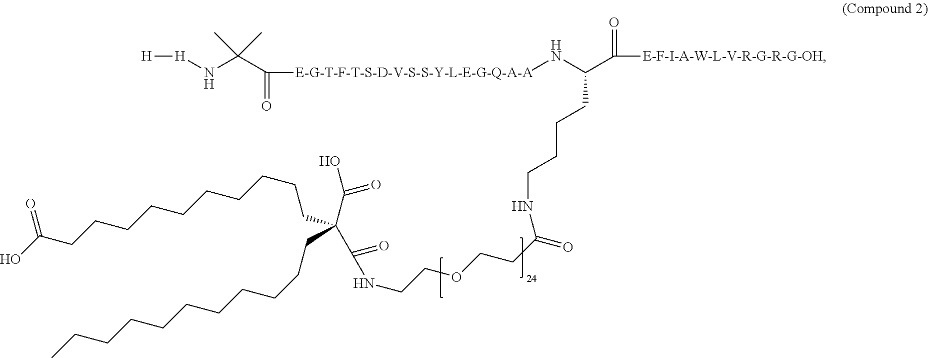

for use in the treatment of a disease or disorder selected from obesity, type 2 diabetes mellitus, insulin resistance, hyperinsulinemia, glucose intolerance, hyperglycemia, diabetic complications (including but not limited to chronic kidney disease), diabetic nephropathy, dyslipidemia, metabolic syndrome, progressive liver disease, cardiovascular disease and neuropathy (in particular peripheral neuropathy, e.g. associated with diabetes.

Another embodiment provides a compound of formula (I) as defined herein or a pharmaceutically acceptable salt thereof, which is:

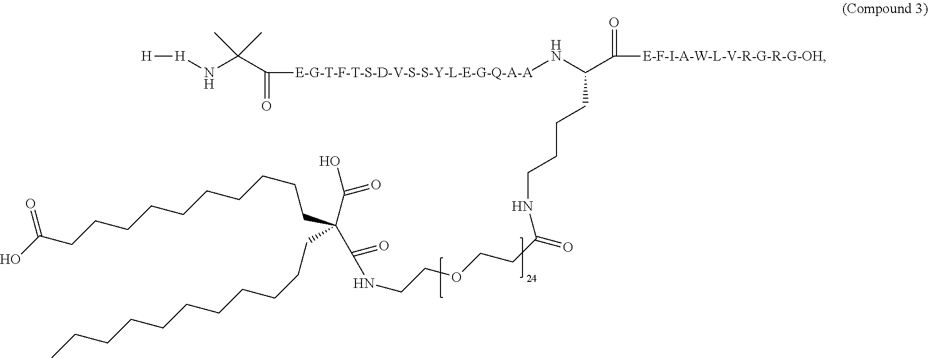

for use in the treatment of a disease or disorder selected from obesity, type 2 diabetes mellitus, insulin resistance, hyperinsulinemia, glucose intolerance, hyperglycemia, diabetic complications (including but not limited to chronic kidney disease), diabetic nephropathy, dyslipidemia, metabolic syndrome, progressive liver disease, cardiovascular disease and neuropathy (in particular peripheral neuropathy, e.g. associated with diabetes.

Another embodiment provides a compound of formula (I) as defined herein or a pharmaceutically acceptable salt thereof, which is:

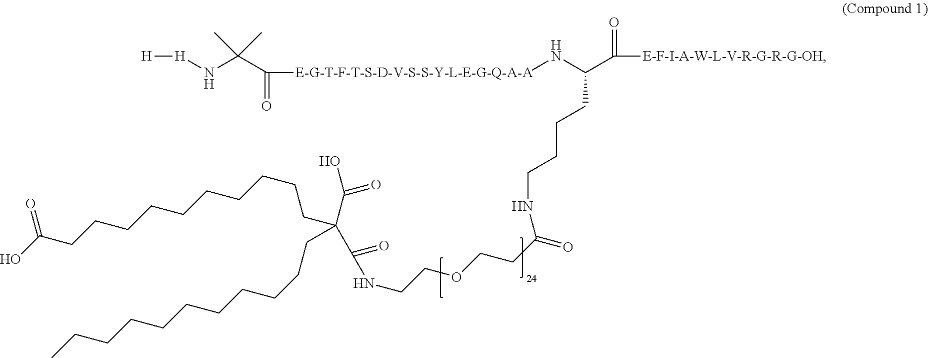

for use in the treatment of a disease or disorder selected from hypertension, atherosclerosis, peripheral arterial disease, stroke, cardiomyopathy, atrial fibrillation, heart failure (for example heart failure with reduced ejection fraction (HFrEF), heart failure with mid-range ejection fraction (HFmrEF)) and heart failure with preserved ejection fraction (HFpEF), coronary heart disease and arrhythmias (for example atrial arrhythmias and ventricular arrhythmias).

Another embodiment provides a compound of formula (I) as defined herein or a pharmaceutically acceptable salt thereof, which is:

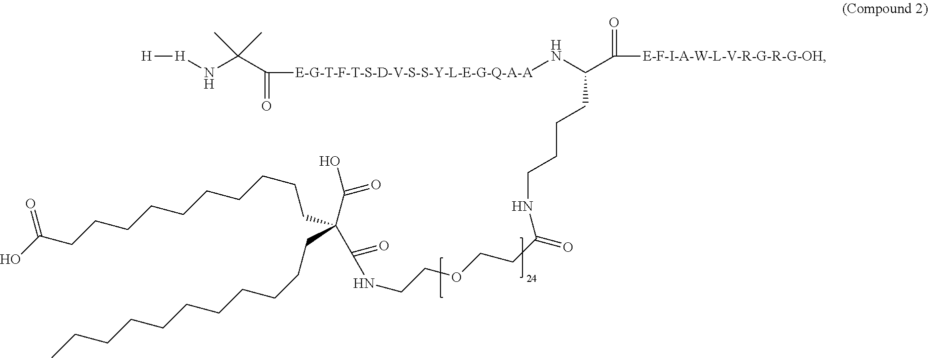

for use in the treatment of a disease or disorder selected from hypertension, atherosclerosis, peripheral arterial disease, stroke, cardiomyopathy, atrial fibrillation, heart failure (for example heart failure with reduced ejection fraction (HFrEF), heart failure with mid-range ejection fraction (HFmrEF)) and heart failure with preserved ejection fraction (HFpEF), coronary heart disease and arrhythmias (for example atrial arrhythmias and ventricular arrhythmias).

Another embodiment provides a compound of formula (I) as defined herein or a pharmaceutically acceptable salt thereof, which is:

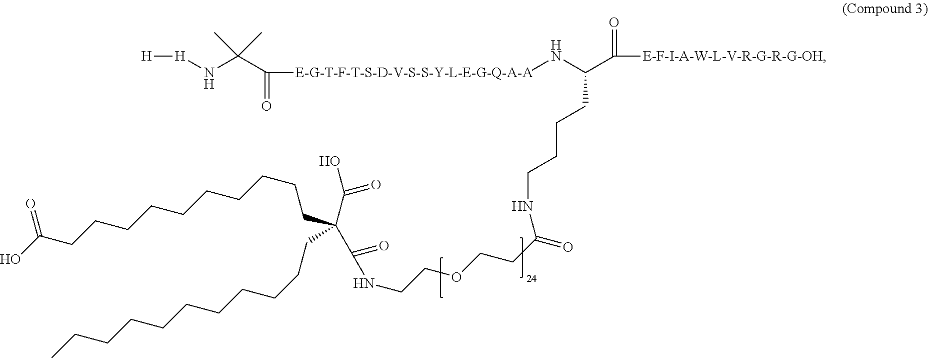

for use in the treatment of a disease or disorder selected from hypertension, atherosclerosis, peripheral arterial disease, stroke, cardiomyopathy, atrial fibrillation, heart failure (for example heart failure with reduced ejection fraction (HFrEF), heart failure with mid-range ejection fraction (HFmrEF)) and heart failure with preserved ejection fraction (HFpEF), coronary heart disease and arrhythmias (for example atrial arrhythmias and ventricular arrhythmias).

### Dosage Forms

The pharmaceutical composition or combination as described herein can be in unit dosage of about 1-100 mg of active ingredient(s) for a subject of about 50-70 kg. The therapeutically effective dosage of a compound, the pharmaceutical composition, or the combinations thereof, is dependent on the species of the subject, the body weight, age and individual condition, the disorder or disease or the severity thereof being treated.

### Combination Aspects

Any compound described herein may be administered either simultaneously with, or before or after, one or more other therapeutic agent. Any compound described herein may be administered separately, by the same or different route of administration, or together in the same pharmaceutical composition as the other agents. A therapeutic agent is, for example, a chemical compound, peptide, peptide conjugates and fusions, antibody, antibody fragment or nucleic acid, which is therapeutically active or enhances the therapeutic activity when administered to a subject in combination with a compound described herein.

Thus, in another aspect, provided herein is a combination, in particular a pharmaceutical combination, comprising (e.g., a therapeutically effective amount of) a compound described herein, or a pharmaceutically acceptable salt thereof, and one or more other therapeutically active agents.

In one embodiment, provided herein is a combination comprising a compound described herein and at least one other therapeutic agent as a combined preparation for simultaneous, separate or sequential use in therapy. In one embodiment, the therapy is the treatment of a disease, disorder or condition selected from the afore-mentioned lists.

Products provided as a combined preparation include a composition comprising a compound described herein and one or more additional therapeutic agent(s) together in the same pharmaceutical composition, or the compound described herein and the other therapeutic agent(s) in separate form, e.g., in the form of a kit.

In one embodiment, provided herein is a pharmaceutical combination comprising a compound described herein and one or more additional therapeutic agent(s). Optionally, the pharmaceutical combination may comprise a pharmaceutically acceptable carrier, as described above.

In one embodiment, provided herein is a kit comprising two or more separate pharmaceutical compositions, at least one of which contains a compound described herein. In one embodiment, the kit comprises means for separately retaining said compositions, such as a container, divided bottle, or divided foil packet. An example of such a kit is a blister pack, as typically used for the packaging of tablets, capsules and the like.

The kit may be used for administering different dosage forms, for example, oral and parenteral, for administering the separate compositions at different dosage intervals, or for titrating the separate compositions against one another. To assist compliance, the kit typically comprises directions for administration.

In the combination therapies described herein, any compound described herein and the other therapeutic agent may be manufactured and/or formulated by the same or different manufacturers.

Moreover, any compound described herein and the other therapeutic may be brought together into a combination therapy: (i) prior to release of the combination product to physicians (e.g., in the case of a kit comprising the compound described herein and the other therapeutic agent); (ii) by the physician themselves (or under the guidance of the physician) shortly before administration; (iii) in the patient themselves, e.g., during sequential administration of the compound described herein and the other therapeutic agent.

Also provided herein is a combination comprising a compound as described herein and one or more additional therapeutic agent for use in a method of treating a disease, disorder or condition selected from any of the afore-mentioned lists.

Also provided herein is the use of a combination comprising a compound as described herein and one or more additional therapeutic agents for treating a disease, disorder or condition selected from any of the afore-mentioned lists.

In one embodiment, the other therapeutic agent may be selected from:
1. Antidiabetic agents, such as insulin, insulin derivatives and mimetics; insulin secretagogues such as the sulfonylureas (e.g. chlorpropamide); or DPPIV (dipeptidyl peptidase IV) inhibitors such as vildagliptin;
2. Hypolipidemic agents such as 3-hydroxy-3-methyl-glutaryl coenzyme A (HMG-CoA) reductase inhibitors, e.g. lovastatin; squalene synthase inhibitors; FXR (farnesoid X receptor) and LXR (liver X receptor) ligands; bile acid sequestrants such as cholestyramine and colesevelam; fibrates; nicotinic acid or aspirin;
3. Anti-obesity agents such as orlistat;
4. Anti-hypertensive agents, e.g. loop diuretics such as ethacrynic acid; angiotensin converting enzyme (ACE) inhibitors such as benazepril; inhibitors of the N^{α}—K-ATPase membrane pump such as digoxin; neutralendopeptidase (NEP) inhibitors; ACE/NEP inhibitors such as omapatrilat; angiotensin II antagonists such as valsartan; angiotensin receptor-neprilysin inhibitors (ARNi) such as sacubitril/valsartan (LCZ696); renin inhibitors such as ditekiren; β-adrenergic receptor blockers such as timolol; inotropic agents such as digoxin; calcium channel blockers such as amlodipine; aldosterone receptor antagonists; or aldosterone synthase inhibitors;
5. Agonists of peroxisome proliferator-activator receptors, such as fenofibrate;
6. Compounds that bind the corticotropin-releasing hormone receptors, such as Urocortin 2.

### EXAMPLES

The disclosure is further illustrated by the following examples and synthesis schemes, which are not to be construed as limiting this disclosure in scope or spirit to the specific procedures herein described. It is to be understood that the examples are provided to illustrate certain embodiments and that no limitation to the scope of the disclosure is intended thereby. It is to be further understood that resort may be had to various other embodiments, modifications, and equivalents thereof which may suggest themselves to those skilled in the art without departing from the spirit of the present disclosure and/or scope of the appended claims.

Compounds of the present disclosure may be prepared by methods known in the art of organic synthesis. In all of the methods it is understood that protecting groups for sensitive or reactive groups may be employed where necessary in accordance with general principles of chemistry. Protecting groups are manipulated according to standard methods of organic synthesis (T. W. Green and P. G. M. Wuts (1999) Protective Groups in Organic Synthesis, 3rd edition, John Wiley & Sons). These groups are removed at a convenient stage of the compound synthesis using methods that are readily apparent to those skilled in the art.

### EXPERIMENTAL SECTION

### Analytical Methods, Materials, and Instrumentation

Unless otherwise noted, reagents and solvents were used as received from commercial suppliers. Proton nuclear magnetic resonance (NMR) spectra were obtained on either Bruker Avance spectrometer or Varian Oxford 400 MHz spectrometer unless otherwise noted. Spectra are given in ppm (δ) and coupling constants, J, are reported in Hertz. Tetramethylsilane (TMS) was used as an internal standard. Chemical shifts are reported in ppm relative to dimethyl sulfoxide (δ 2.50), methanol (δ 3.31), chloroform (δ 7.26) or other solvent as indicated in NMR spectral data. A small amount of the dry sample (2-5 mg) is dissolved in an appropriate deuterated solvent (1 mL). The chemical names were generated using ChemBioDraw Ultra v17 from CambridgeSoft.

### Abbreviations

AC₅₀ concentration at half-maximal compound effect
ACN acetonitrile
A_{inf} plateau value of Hill curve at high concentrations
A₀ plateau value of Hill curve at low concentrations
Aib α-aminoisobutyric acid
ALS autosampler
AUC_{inf} area under the plasma concentration-time curve from time zero to infinity
br broad
BSA bovine serum albumin
BW body weight
cAMP cyclic adenosine monophospate
cat # catalog number
CHO Chinese Hamster Ovary cells
Cmax maximum plasma concentration
CO₂ carbon dioxide
cynoGLP1R cynomolgus glucagon-like peptide 1 receptor
d doublet
dd doublet of doublets
DCC N,N′-dicyclohexylcarbodiimide
DCM dichloromethane
DCU N,N′-dicyclohexylurea
DEA N,N-diethylaniline
DERET dissociation enhanced resonance energy transfer
DIEA/DIPEA diethylisopropylamine
DIO diet-induced obese
DMEM Dulbecco's Modified Eagle Media
DMF N,N-dimethylformamide
DMSO dimethylsulfoxide
DSC N,N′-disuccinimidyl carbonate
DMA dimethylacetamide
DMAP 4-(N,N-dimethylamino)pyridine
EA enzyme acceptor
EC effective concentration
EC₀ effective concentration of a compound that gives no response
EC₅₀ effective concentration of a compound that gives a half-maximal response
EC₁₀₀ effective concentration of a compound that gives a maximal (100%) response
Eₘₐₓ efficacy: maximum response achievable from a dosed agent
EDC or EDCI N-ethyl-N′-(3-dimethylaminopropyl)carbodiimide
EDTA ethylenediaminetetraacetic acid
Ex9-39 exendin 9-39
ELSD evaporative light scattering detector
equiv equivalents
ESI electrospray ionization
EtOAc ethyl acetate
FBS fetal bovine serum
FI food intake
Fmoc 9-Fluorenylmethoxycarbonyl
FRET fluorescence resonance energy transfer
g gram(s)
GLP1 glucagon-like peptide 1
GLP1R glucagon-like peptide 1 receptor
GPCR G-protein coupled receptor
G418 geneticin, a selection antibiotic
Grubbs II Dichloro[1,3-bis(2,4,6-trimethylphenyl)-2-imidazolidinylidene](benzylidene)(tricyclohexylphosphine) ruthenium(II)
h hour(s)
HATU (1-[Bis(dimethylamino)methylene]-1H-1,2,3-triazolo[4,5-b]pyridinium 3-oxid hexafluorophosphate
HDF high fat diet
HESI heated electrospray ionization
hGLP1R human glucagon-like peptide 1 receptor
HPLC high-pressure liquid chromatography
HTRF homogenous time resolved fluorescence
IBMX 3-isobutyl-1-methylxanthine
kg kilogram(s)
L liter
LCMS liquid chromatography and mass spectrometry
MeOH methanol
MS mass spectrometry
MTBE methyl tert-butyl ether
m multiplet
mg milligram(s)
min minutes
mL milliliter
mmol millimole
mM millimolar
m/z mass to charge ratio
nM nanomolar
nmol nanomole
NMP N-methyl-2-pyrrolidinone
NMR nuclear magnetic resonance
NPLC normal-phase liquid chromatography
p pentet
Pbf 2,2,4,6,7-Pentamethyldihydrobenzofuran-5-sulfonyl-
PBS phosphate-buffered saline
Pd/C palladium on carbon
PEG polyethylene glycol
PK pharmacokinetic
PD pharmacodynamic
ppm parts per million
QC quality control
QD once a day
03D once every 3 days
Q1W once a week
RCF relative centrifugal force
RPM revolutions per minute
R, retention time
RT room temperature
rotovap rotary evaporator
s singlet
s.c. or SC subcutaneous
sec seconds
SEM standard error of the mean
SFC supercritical fluid chromatography
SM starting material(s)
t triplet
TEA triethylamine
TFA trifluoroacetic acid
THF tetrahydrofuran
Tₘₐₓ time taken to reach maximal plasma concentration
T_{1/2} half-life
v/v volume/volume
μg microgram
μL microliter
μM micromolar

### Biological Assays and Data

Compounds described herein were tested in the following cellular assays that measures the intracellular cAMP concentration. The cAMP is generated by the activation of GLP1R. The data obtained is shown in Tables 1-3. EC₅₀ is defined as the concentration of the compound that leads to half of the maximum response (after baseline correction). Eₘₐₓ is defined as the maximum response observed for the test compound, normalized to the maximum response observed for the endogenous ligand (GLP1(7-36)) to GLP1R.

Human GLP1R cAMP Agonist Assay

The agonist activity of compounds was determined using the GloSensor™ cAMP Assay (Promega Corp.), which measures changes in the intracellular concentration of cAMP after ligand activation of GPCRs. The assay uses a biosensor encoded by pGloSensor™-22F cAMP plasmid (Promega, cat #E2301) with cAMP binding domains fused to a mutant form of Photinus pyralis luciferase. Binding to cAMP causes conformational changes that promote large increases in light output, which can be measured by a luminescence detector. HEK293-SNAP-hGLP1R-GloSensor cells stably overexpressing the human GLP1 receptor (hGLP1R) and pGloSensor™-22F were seeded in white 384-well poly-D-Lysine coated plates (Greiner Bio One, cat #781945) in CO₂-independent media (Gibco cat #18045-088 with 1.0% FBS, 2 mM L-glutamine, penicillin and streptomycin) and incubated overnight at 37° C., 5% CO₂ with humidity. The assay was started the following morning by adding an equal volume of CO₂-independent media containing 4% v/v dilution of the GloSensor substrate (Promega, cat #E1291) to all wells. The cell plate was incubated at RT for 2 h in the dark. The Biomek i7 (Beckman Coulter) instrument was used for the liquid handling steps. To generate duplicate dose response curves, 3-fold serially diluted compounds were added in to the cell assay plate to a final volume of 60 μL with final concentrations ranging from 100 nM through 0.03 pM in CO₂-independent media containing 0.1% BSA, 0.5 mM IBMX and 0.4% DMSO. EC₁₀₀ control wells containing GLP1(7-36) peptide (Bachem, cat #H-6795) at a final concentration of 2 nM and EC₀ control wells containing no peptide were tested concurrently in the same plate and using the same assay buffer as the tested compounds. This plate was incubated at RT in the dark for 12 min after adding the compounds to the cells. Luminescence was then measured with an Envision 2104 Multilabel reader with “TRF Light Unit, 337 nm” (PerkinElmer) using the Ultra-Sensitive protocol setting “384-well US luminescence detector” with the 384-well luminescence aperture, 0.1 sec per well. cAMP activity was calculated as percent of the GLP1(7-36) EC₁₀₀ control wells: [(sample signal−mean EC₀ signal)/(mean EC₁₀₀ of GLP1(7-36) signal−mean EC₀ signal)}*100. Curve fitting for EC₅₀ determinations was performed in the Helios module of the software package DAVID. The 4-parameter logistic model, Hill slope was used: y=A_{inf}+(A₀−A_{inf})/(1+(x/AC₅₀₎^{Hill Slope}), where y is the functional response; x is the compound concentration; A₀ is the minimum value (at 0 dose); A_{inf} is the maximum value (at infinite dose); AC₅₀ corresponds to the point of inflection (i.e., the point on the sigmoid shaped curve halfway between A₀ and A_{inf}). The EC₅₀ value was represented by the AC₅₀ value calculated from Helios in μM. Eₘₐₓ is the maximal activity detected within the concentration range, derived from the fitted curve.

### Generation of the HEK293-SNAP-hGLP1R Cell Line

327 μL of Opti-MEM medium (Gibco, cat #31985-062) was mixed with 12 μL of FuGENE® HD (Promega, cat #E2311) and incubated at RT for 5 min. Then 8.2 μL (4 μg, 0.485 μg/μL solution) of pSNAP-hGLP1R plasmid (Cisbio, cat #PSNAP-GLP1) encoding human GLP1R (NCBI Reference Sequence: NM_002062.3) fused with a SNAP tag was added in to the Fugene HD/Opti-MEM mix, and incubated at RT for 20 min. A suspension of HEK293 cells (ATCC® CRL-1573™) was prepared at 800,000 cells/mL. Then, the plasmid/FuGene HD mixture was added to 8 mL of cells and mixed gently. 2 mL of the new mix were added to 4 wells in a 6-well plate and 2 mL of un-transfected cells were added to two wells as control. The plate was incubated at 37° C. until 100% confluence. The antibiotic selection [800 μg/mL G418 (Geneticin, Gibco, cat #10131-035)] was done after cell trypsinization at a dilution of 2500 cells/mL. 1 mL cell suspension was added to 20 mL selection medium in a 10 cm culture dish (2500 cells in total) and in parallel, 4 mL diluted cell suspension were added to 20 mL selection medium in a 10 cm culture dish (10000 cells in total). The rest of the cells were cultured in a T150 flask. In addition, HEK293 cells were cultured in a T75 flask in selection medium as negative control. Finally, single clones were picked from a 10 cm culture dish and cultured until there were enough cells for gene expression analysis and an HTRF cAMP assay. Clone 2 showed the highest GLP1R-dependent cAMP response and was expanded for the generation of the GloSensor stable cell line.

### Generation of HEK293-SNAP-hGLP1R-GloSensor Stable Cell Line

The HEK293 cells stably overexpressing SNAP-hGLP1R (described above) were plated at a density of 3 million cells in a 10 cm dish containing 17 mL of DMEM complete growth medium (Gibco, cat #11965-092)+10% Fetal Bovine Serum (FBS, Gibco, cat #16140-071). The following day, cells were transfected as follows. The DNA complex was prepared as 0.020 μg/μL pGloSensor™-22F cAMP plasmid (Promega, cat #E2301; GenBank® accession is GU174434) by adding 37 μg of plasmid DNA in 1758 μL Opti-MEM solution. Then, 112 μL of FuGENE® HD reagent was added to that by mixing carefully. After 5-10 min incubation at RT, 850 μL of complex per well was added to the cells, and mixed thoroughly. After 24 h incubation at 37° C., 5% CO₂ with humidity, the media was removed and cells were rinsed with PBS. Then, the selection medium [600 μg/mL G418 and 600 μg/mL hygromycin B (Gibco, cat #10687010)] was added. The medium was changed twice a week until no more dead cells were observed. Once cell clones were visible, single cells were isolated by pipetting up and down after addition of 10 μL of 0.05% Trypsin-EDTA solution. These single cell-derived clones were then cultured in six well plates with selection medium (600 μg/mL G418+600 μg/mL hygromycin B) until enough cells were available to be tested for cAMP agonist response in the GloSensor luminescence assay described herein. The HEK293-SNAP-hGLP1R stable cell clone that yielded the desired response was used for human GLP1R cAMP agonist assay. These data are indicative of the relative potency of the tested compounds.

TABLE 1
hGLP1R CAMP Assay Summary

| | EC₅₀ | EC₅₀ | Eₘₐₓ | Eₘₐₓ | | |
|---|---|---|---|---|---|---|
| | mean | SEM | mean | SEM | | |
| compound | (μM) | (μM) | (%) | (%) | n | solvent |
| Compound 1 | 1.72E−05 | 1.32E−06 | 105 | 4 | 6 | DMSO |
| Compound 2 | 1.99E−05 | 2.06E−06 | 115 | 2 | 5 | DMSO |
| Compound 3 | 1.51E−05 | 9.80E−07 | 108 | 1 | 6 | DMSO |
| Compound 4 | 2.94E−05 | 1.30E−05 | 119 | 2 | 3 | DMSO |
| Compound 5 | 8.83E−05 | 3.53E−06 | 111 | 2 | 3 | DMSO |
| Compound 6 | 5.73E−06 | 2.31E−06 | 111 | 7 | 3 | DMSO |
| Compound 7 | 2.45E−05 | 2.15E−06 | 112 | 5 | 3 | DMSO |
| Compound 8 | 1.05E−04 | 1.06E−05 | 106 | 0 | 2 | DMSO |
| Compound 9 | 2.02E−04 | 6.72E−06 | 107 | 2 | 3 | DMSO |
| semaglutide** | 1.44E−05 | 7.50E−07 | 109 | 3 | 6 | DMF |
| GLP1(7-36) | 3.71E−06 | 2.60E−07 | 109 | 1 | 6 | PBS + |
| | | | | | | 0.1% |
| | | | | | | BSA |

**semaglutide acetate salt (Tables 1-6) was purchased from Bachem (Catalogue No. H-7894) and was dissolved in DMF as stated in the row “solvent”.

Cynomolgus GLP1R cAMP Agonist Assay

The agonist activity of the described Compounds was further tested using an HTRF cAMP assay (CisBio, cat #62AM4PEC), which measures changes in the intracellular concentration of cAMP after ligand activation of GPCRs. This assay is based on a competitive format involving a specific anti-cAMP monoclonal antibody labeled with Eu³⁺ cryptate (donor fluorophore) and cAMP coupled to d2 (acceptor fluorophore). This enables the direct characterization of compounds acting on G protein-coupled receptors in cells. Native cAMP produced by cells competes with d2-labeled cAMP for binding to anti-cAMP antibody-Eu³⁺ cryptate. HEK293-cynoGLP1R F6 cells stably overexpressing cynomolgus GLP1 receptor (cynoGLP1R) were seeded in white 384-well poly-D-Lysine coated plates (Greiner Bio One, cat #781945) at 5000 cells/well in DMEP complete medium (Gibco, cat #11965-092, 10% Heat Inactivated FBS, 0.5 mg/ml Geneticin; Gibco Life Technologies, cat #10131027) and incubated overnight at 37° C., 5% CO₂ with humidity. The assay was performed the next day. Peptides were diluted in Stimulation Buffer [1×HBSS (Life Technologies, cat #14065-056), 20 mM HEPES (Life Technologies, cat #15630), 0.1% BSA (Sigma, cat #A0281) and 0.5 mM IBMX]. To generate triplicate dose response curves, 3-fold serially diluted compounds (at 2 times concentration) were diluted in DMSO. Cells were washed with ELx405 Select, BioTek plate washer, leaving 10 μL/well Assay Buffer [1×HBSS (Life Technologies, cat #14065-056), 20 mM HEPES (Life Technologies, cat #15630)]. Plates were centrifuged briefly and 10 μL of 2 times diluted peptides were added per well. Plates were again centrifuged briefly and incubated at RT for 30 min. 20×d2 and Eu³⁺ cryptate were diluted in Lysis Buffer provided with the kit. After a 30 min incubation with peptides, 10 μL of diluted d2 were added per well followed by 10 μL of diluted Eu³⁺ cryptate. Plates were covered with black lids and incubated at RT for 1 h following brief centrifugation. The HTRF signal was then measured with an Envision 2104 Multilabel reader (Perkin Elmer) with fluorescence emission set at two different wavelengths (665 nm and 620 nm). Curve fitting for EC₅₀ determinations was performed in the Helios module of the software package DAVID. 4-Parameter Logistic curve fitting was performed on the section of the plate where the standard curve compound was placed to obtain the 4 standard parameters: std_crv_ac50 (standard curve AC₅₀), std_crv_a0 (standard curve A₀), std_crv_ainf (standard curve A_{inf}), std_crv_hill (standard curve Hill slope). Then, these 4 parameter values were used to apply the standard curve transformation to each well using formula: y=std_crv_ac50*[(X−std_crv_a0)/(std_crv_ainf−X)]\hat{ }(1/std_crv_hill). y is the functional response; x is the compound concentration. 4-Parameter Logistic curve fitting was performed on the transformed data to obtain the AC₅₀ in μM for all tested compounds, which represents the EC₅₀ value for this assay. Eₘₐₓ was expressed as percent of the GLP1(7-36) EC₁₀₀: [(sample Aₘₐₓ−sample A₀)/(GLP1(7-36) Aₘₐₓ−GLP1(7-36) A₀)]*100.

### Generation of HEK293-cynoGLP1R Stable Cell Line

HEK293 cells were plated the day before transfection at a density of 1×10⁶ cells in 8 mL of DMEM complete growth medium+10% FBS in a T25 flask. The following day, cells were transfected as follows. The DNA complex was prepared as 0.020 μg/μl by adding 8.8 μg pcDNA3.1 (+) Neo plasmid encoding cyno GLP1R cDNA [codon optimized, GeneArt (Thermo Fisher Scientific); NCBI Reference Sequence: NP_001274592] in 414 μL of OptiMEM solution. Then, 26 μL of FuGENE® HD reagent was added to that by mixing carefully. After 5-10 min incubation at RT, 400 μL of complex per well were added to the cells, and mixed thoroughly. After 48 h incubation at 37° C., 5% CO₂ with humidity cells were transferred to a 15 cm dish in the presence of 0.5 mg/mL geneticin. The HEK293T-cynoGLP1R stable cell clone that showed the highest activity in a functional cAMP assay (Clone F6) was selected for further analysis in a cyno GLP1R cAMP cellular agonist assay.

These data are indicative of the relative potency of the tested compounds.

TABLE 2
cynoGLP1R CAMP Assay Summary

| | EC₅₀ | EC₅₀ | Eₘₐₓ | Eₘₐₓ | | |
|---|---|---|---|---|---|---|
| | mean | SEM | mean | SEM | | |
| compound | (μM) | (μM) | (%) | (%) | n | solvent |
| Compound 1 | 1.59E−04 | 2.08E−05 | 111 | 4.5 | 6 | DMSO |
| Compound 2 | 2.18E−04 | 2.41E−05 | 119 | 7.3 | 7 | DMSO |
| Compound 3 | 2.94E−04 | 3.63E−05 | 108 | 4.2 | 3 | DMSO |
| Compound 4 | 4.68E−04 | 8.05E−05 | 112 | 4.2 | 5 | DMSO |
| Compound 5 | 5.77E−04 | 1.09E−04 | 103 | 4.0 | 3 | DMSO |
| Compound 6 | 6.61E−05 | 1.51E−05 | 108 | 6.1 | 3 | DMSO |
| Compound 7 | 3.18E−04 | 2.05E−05 | 108 | 8.4 | 4 | DMSO |
| Compound 9 | 2.24E−03 | 3.81E−04 | 111 | 4.6 | 3 | DMSO |
| Semaglutide** | 4.91E−05 | 3.4E−06 | 110 | 3.3 | 6 | DMF |
| GLP1(7-36) | 2.17E−05 | 2.48E−06 | 100 | 0 | 6 | PBS + |
| | | | | | | 0.1% |
| | | | | | | BSA |

Mouse GLP1R cAMP Agonist Assay

The cAMP agonist activity of compounds was tested using a similar procedure as the cynomolgus GLP1R cAMP assay (see above), except for the fact that HEK293-mGLP1R CRE-Luc (Clone C3) cells stably overexpressing mouse GLP1 receptor (mGLP1R) were used (generation described below).

### Generation of HEK293-mGLP1R CRE-Luc Stable Cell Line

HEK293T CRE-Luc cells were plated at a density of 3×10⁶ cells in 17 mL of DMEM complete growth medium+10% FBS in a 10 cm dish. The following day, cells were transfected as follows. The DNA complex was prepared as 0.020 μg/μL by adding 37 μg of plasmid DNA encoding mouse GLP1R cDNA (GeneCopoeia, cat #EX-Mm23901-M67; NCBI Reference Sequence: NM_021332.2) in 1758 μL Opti-MEM solution. Then, 112 μL of FuGENE® HD reagent were added to that by mixing carefully. After 5-10 min incubation at RT, 850 μL of DNA complex per well were added to the cells, and mixed thoroughly. After 24 h incubation at 37° C., 5% CO₂ with humidity, the media was removed; and the cells were rinsed with PBS and split. Then, selection medium [2 μg/mL puromycin (Corning, cat #61-385-RA) and 100 μg/mL hygromycin (Gibco, cat #10687010)] was added. The medium was changed thrice a week until no more dead cells were observed. Once cell clones were visible, single cells were isolated. The HEK293T-mGLP1R-CRE-Luc stable cell clone that showed the maximum gene expression (Clone C3) was used for the mouse GLP1R cAMP cellular agonist assay.

These data are indicative of the relative potency of the tested compounds.

TABLE 3
mGLP1R CAMP Assay Summary

| | EC₅₀ | EC₅₀ | Eₘₐₓ | Eₘₐₓ | | |
|---|---|---|---|---|---|---|
| | mean | SEM | mean | SEM | | |
| compound | (μM) | (μM) | (%) | (%) | n | solvent |
| Compound 1 | 1.26E−04 | 1.52E−05 | 111 | 3.1 | 12 | DMSO |
| Compound 3 | 1.69E−04 | 2.09E−05 | 110 | 3.6 | 8 | DMSO |
| Compound 2 | 1.55E−04 | 1.42E−05 | 117 | 1.9 | 13 | DMSO |
| Compound 5 | 5.44E−04 | 8.68E−05 | 107 | 3.5 | 4 | DMSO |
| Compound 4 | 4.28E−04 | 1.27E−04 | 118 | 4.7 | 6 | DMSO |
| Compound 9 | 6.86E−04 | 1.80E−04 | 109 | 3.5 | 4 | DMSO |
| Compound 6 | 3.40E−05 | 2.76E−05 | 104 | 3.1 | 6 | DMSO |
| Compound 7 | 1.55E−04 | 2.65E−05 | 96 | 3.9 | 6 | DMSO |
| semaglutide** | 3.37E−05 | 3.08E−05 | 106 | 2.8 | 5 | DMF |
| GLP1(7-36) | 7.78E−06 | 1.04E−06 | 100 | 0 | 8 | PBS + |
| | | | | | | 0.1% |
| | | | | | | BSA |

### Human GLP1R β-Arrestin Recruitment Assay

The extent to which agonists recruited β-arrestin was measured using the PathHunter® β-arrestin assay (DiscoverX). This assay measures binding of β-arrestin to the receptor using an enzyme complementation approach. Two inactive portions of a β-galactosidase enzyme (termed Prolink and Enzyme Acceptor, or ‘EA’) are tagged so that the human GLP1R (hGLP1R) contains the Prolink portion and β-arrestin contains the EA portion. When β-arrestin is recruited to the receptor the enzyme becomes active and generates luminescence in the presence of a chemiluminescent substrate (PathHunter® Detection Kit, DiscoverX cat #93-0001). Luminescence can be measured on a relevant detector. CHO-hGLP1R-β-arrestin cells stably overexpressing hGLP1R with a Prolink tag and β-arrestin with an EA tag were seeded at 20 μL per well in white 384-well poly-D-Lysine coated plates (Greiner Bio One, cat #781945) in Plating Reagent 2 (DiscoverX, cat #93-0563R2A), and incubated overnight at 37° C., 5% CO₂ with humidity. The following day, agonists were prepared at 5 times the final required concentration. To generate triplicate dose response curves, compounds were serially diluted 3-fold in assay buffer (HBSS, 10 mM Hepes and 0.1% BSA), then added to the cell assay plate to a final volume of 25 μL and final top concentrations starting at 3 μM or less, depending on the compound. EC₁₀₀ control wells containing GLP1(7-36) peptide (Bachem, cat #H-6795) at a final concentration of 1 μM and EC₀ control wells containing no compound were tested concurrently in the the same plate and using the same assay buffer as the tested compounds. The plate was incubated at 37° C., 5% CO₂ with humidity for 2 h after adding the compounds to the cells. Then the detection reagent was prepared (19 parts cell assay buffer, 5 parts substrate reagent 1, and 1 part substrate reagent 2 as per manufacturers recommendations, DiscoverX cat #93-0001), and 12 μL were added per well to the cell assay plate. The plate was incubated for an additional hour in the dark at RT. Luminescence was then measured with an Envision 2104 Multilabel reader with “TRF Light Unit, 337 nm” (Perkin Elmer) using the Ultra-Sensitive protocol setting “384-well US luminescence detector” with the 384-well luminescence aperture, 0.1 sec per well. β−arrestin recruitment was calculated and expressed as percent of the GLP1(7-36) EC₁₀₀ control wells: [(sample signal−mean EC₀ signal)/(mean EC₁₀₀ of GLP1(7-36) signal−mean EC₀ signal)]*100 using Microsoft Excel. Curve fitting for EC₅₀ determinations was performed using GraphPad Prism. The 4-parameter logistic model, Hill slope was used: Y=Bottom+(Top−Bottom)/(1+10\hat{ }((Log EC₅₀−X)*Hill Slope)), where Y is the functional response; X is the compound concentration; bottom is A₀ or the minimum value (at 0 dose); top is A_{inf} or the maximum value (at infinite dose); EC₅₀ is the point of inflection (i.e., the point on the sigmoid shaped curve halfway between A₀ and A_{inf}). The EC₅₀ value was calculated in μM. Eₘₐₓ is the maximal activity detected within the concentration range, derived from the fitted curve relative to GLP1 (7-36).

### Generation of CHO-hGLP1R-β-Arrestin Cell Line

PathHunter® CHO-K1-EA parental cells (DiscoverX, cat #93-0164) were plated at a density of 2×10⁶ cells per T75 cm² flask in 22 mL of complete medium (AssayComplete Cell Culture kit 107, DiscoverX, cat #92-3107G). The following day, the medium was replaced with 22 mL of fresh medium with no antibiotics and cells were transfected as follows.

Plasmid/Fugene® HD Transfection mix was prepared in Opti-MEM media (3:1 Ratio of Reagent:DNA). 25 μg (34 μL) of pCMV-PK1-GLP1R plasmid [(DiscoverX pCMV PK vector bundle, cat #93-0491 with sequence inserted encoding full-length human GLP1R-NCBI Reference Sequence: NM_002062, synthesized by GeneArt (Thermo Fisher Scientific)], was added to 1129 μL Opti-MEM for a total volume of 1163 μL. Then, 74 μL of FuGENE® HD Reagent was added by mixing carefully. After 5-10 min incubation at RT, 1125 μL of complex solution were added to the cells and incubated for 48 h at 37° C. Subsequently, the medium was removed and selection medium containing 300 μg/mL hygromycin (Gibco, cat #10687010) and 500 μg/mL geneticin (Gibco, cat #10131035) was added. The medium was changed every 2-3 days until no more dead cells were observed. Cells were detached, re-suspended at 300000 cells/mL and strained with 40 μm strainer. The cells were then FACS sorted using Aria G instrument into single cells in black, clear bottom poly-D-lysine coated 96-well plates in 100 μL medium. Medium was changed every 2-3 days by removing up to 80 μL and adding fresh medium containing selection antibiotics. Surviving single clones were expanded and tested. Single clone 1 was selected for the R-arrestin assay based on optimal signal and curve profile.

TABLE 4
β-arrestin Summary

| | EC₅₀ | EC₅₀ | Eₘₐₓ | Eₘₐₓ | | |
|---|---|---|---|---|---|---|
| | mean | SEM | mean | SEM | | |
| compound | (μM) | (μM) | (%) | (%) | n | solvent |
| Compound 1 | 0.192 | 0.034 | 92 | 1.8 | 7 | DMSO |
| Compound 2 | 0.133 | 0.021 | 94 | 5.1 | 4 | DMSO |
| Compound 3 | 0.149 | 0.030 | 91 | 4.5 | 4 | DMSO |
| Compound 4 | 0.235 | 0.004 | 84 | 10.1 | 4 | DMSO |
| Compound 5 | 0.254 | 0.058 | 77 | 6.1 | 3 | DMSO |
| Compound 6 | 0.070 | 0.006 | 75 | 2.6 | 3 | DMSO |
| Compound 7 | 0.178 | 0.015 | 82 | 12.2 | 3 | DMSO |
| Compound 9 | >2 | 0.000 | 37 | 3.5 | 3 | DMSO |
| Semaglutide** | 0.036 | 0.004 | 67 | 3.5 | 3 | DMF |
| GLP1(7-36) | 0.007 | 0.001 | 100 | 0.0 | 7 | PBS + |
| | | | | | | 0.1% |
| | | | | | | BSA |

Data assessed in the β-arrestin assays may correlate with gastrointestinal tolerability (reduction of nausea/emesis) of the compounds described herein in an inverse manner, i.e., the less active the compound is in the β-arrestin assay, the more tolerable it may be. [see e.g. Jones et. al. Nat. Commun. 2018, 9, 1602.]

### Human GLP1R DERET Internalization and Recycling Assays

The extent to which agonists internalize or allow recycling of the human GLP1R was determined based on an optimized version of a RealTime FRET-based ‘DERET’ (Dissociation Enhanced Resonance Energy Transfer) assay. The technology relies on labeling of the SNAP-tagged GPCR with a SNAP-Lumi-Terbium (donor fluorophore, Cisbio, cat #SSNPTBD). The compounds are incubated with the cells over-expressing the GPCR of interest in the presence of an excess of fluorescein (acceptor fluorophore). When the GPCR is on the cell surface, the donor signal is quenched by the acceptor and the donor/acceptor ratio is low. As the GPCR internalizes, the donor signal is no longer quenched, and the acceptor is no longer excited so the donor/acceptor ratio increases. The addition of an excess of antagonist blocks further receptor internalization, allowing the receptor to recycle back to the membrane leading to a subsequent reduction in the donor/acceptor ratio. HEK293-SNAP-hGLP1R-GloSensor cells (stably overexpressing SNAP-tagged hGLP1R) were seeded overnight in white 384-well poly-D-Lysine coated plates (Greiner Bio One, cat #781945) in regular DMEM growth medium (Gibco, cat #11965-092, 10% heat-inactivated FBS, 10 mM HEPES, 1× penicillin/streptomycin, 0.5 mg/mL geneticin (Gibco, cat #10131-035) and 0.25 mg/mL hygromycin B (Invitrogen, cat #10687010). On the assay day, cell medium was removed and 100 nM SNAP-Lumi-Tb reagent was added in Opti-MEM solution. The cells were incubated at 37° C. for 1 h. Cells were washed using a plate washer in assay buffer [1×HBSS (10× Gibco, cat #14065-056), 20 mM Hepes (Gibco, cat #15630-080), 1 mM CaCl₂ (Fluka, cat #21114-1 L), 1 mM MgCl₂ (Ambion, cat #AM9530G) pH7.4], and 20 μL buffer with 0.1% BSA was added to each well. After leaving cells to equilibrate for ˜15 min at 37° C., 10 μl of Fluorescein (sodium salt, Sigma, cat #F6377, diluted in buffer) was added at 25 μM final concentration. To generate triplicate dose response curves, compounds were serially diluted 3-fold in assay buffer, then added to the cell assay plate to a final volume of 40 μL and final top concentrations starting at 3 μM or less (depending on the case). In the same plate and assay buffer as the tested compounds, a GLP1(7-36) peptide (Bachem, cat #H-6795) control curve was included at a final top concentration of 1 μM in order to establish EC₁₀₀. EC₀ wells with buffer only were also included. The plate FRET fluorescence was measured immediately using a Perkin Elmer Envision with LANCE/DELFIA D400 single mirror, excitation filter X320, and emission filters M615_203 (donor emission) and M515 (acceptor emission), and then measured every 30 min. Peak Internalization was reached at 120 min, at which point 10 μM (final) Exendin 9-39 (Bachem, cat #H8740, GLP1R antagonist) was added to all wells in order to block agonist binding further. The measurements were continued for additional 180 min in order to establish how well the receptor recycled back to the membrane. Plates were kept at 37° C. between reads. Data was expressed as the ratio of donor/acceptor emissions using Microsoft Excel and plotted in GraphPad Prism. In order to determine EC₅₀ and Eₘₐₓ for internalization, data was calculated and expressed as percent of the GLP1(7-36) EC₁₀₀ control wells: [(sample signal−mean EC₀ signal)/(mean EC₁₀₀ of GLP1(7-36) signal−mean EC₀ signal)]*100 using Microsoft Excel. Curve fitting for EC₅₀ determinations was performed using GraphPad Prism. The 4-parameter logistic model, Hill slope was used:

Y=Bottom+(Top−Bottom)/(1+10\hat{ }((Log EC₅₀−X)*Hill Slope)),

where Y is the functional response; X is the compound concentration; bottom is A₀ or the minimum value (at 0 dose); top is A_{inf} or the maximum value (at infinite dose); EC₅₀ is the point of inflection (i.e., the point on the sigmoid shaped curve halfway between A₀ and A_{inf}). The EC₅₀ value was calculated in μM. Eₘₐₓ is the maximal activity that was measured within the concentration range, derived from the fitted curve relative to GLP1(7-36). To determine receptor recycling parameters, relative Eₘₐₓ was calculated at each time point post-Ex9-39 addition and a curve fitted over time using the 4-parameter sigmoidal fit. Using this model, we determined a T_{1/2} rate at which the receptor recycled back to the membrane. We also determined a maximum percentage of receptor recycled as a proportion of the amount internalized initially.

TABLE 5
Internalization Assay Summary

| | EC₅₀ | EC₅₀ | Eₘₐₓ | Eₘₐₓ | | |
|---|---|---|---|---|---|---|
| | mean | SEM | mean | SEM | | |
| compound | (μM) | (μM) | (%) | (%) | n | solvent |
| Compound 1 | 0.535 | 0.05 | 88 | 5.4 | 3 | DMSO |
| Compound 2 | 0.331 | 0.07 | 96 | 3.0 | 3 | DMSO |
| Compound 3 | 0.372 | 0.08 | 93 | 6.7 | 3 | DMSO |
| Compound 4 | 0.056 | 0.01 | 100 | 3.2 | 3 | DMSO |
| Compound 5 | 0.079 | 0.03 | 105 | 4.4 | 3 | DMSO |
| Compound 6 | 0.153 | 0.06 | 92 | 8.4 | 3 | DMSO |
| Compound 7 | 0.367 | 0.08 | 77 | 4.8 | 3 | DMSO |
| Compound 9 | >1 | 0.00 | 22 | 0.0 | 3 | DMSO |
| semaglutide** | 0.070 | 0.01 | 91 | 5.9 | 4 | DMF |
| GLP1(7-36) | 0.027 | 0.00 | 100 | 0.0 | 8 | PBS + |
| | | | | | | 0.1% |
| | | | | | | BSA |

TABLE 6
Recycling Assay Summary

| | T_{1/2} | T_{1/2} | Receptor | Receptor | | |
|---|---|---|---|---|---|---|
| | mean | SEM | recycled | recycled | | |
| compound | (min) | (min) | mean (%) | SEM (%) | n | solvent |
| Compound 1 | 62.3 | 3.7 | 65 | 6.5 | 3 | DMSO |
| Compound 2 | 71.3 | 5.2 | 68 | 4.6 | 3 | DMSO |
| Compound 3 | 74.2 | 0.5 | 69 | 5.8 | 3 | DMSO |
| Compound 4 | 105.5 | 6.4 | 65 | 2.6 | 3 | DMSO |
| Compound 5 | 135.2 | 9.7 | 62 | 5.5 | 3 | DMSO |
| Compound 6 | 69.1 | 0.2 | 70 | 4.8 | 3 | DMSO |
| Compound 7 | 76.4 | 5.6 | 71 | 4.0 | 3 | DMSO |
| Compound 9 | NC | NC | NC | NC | 3 | DMSO |
| Semaglutide** | 100.6 | 5.1 | 64 | 3.9 | 3 | DMF |
| GLP1(7-36) | 61.1 | 1.9 | 41 | 1.7 | 6 | PBS + 0.1% |
| | | | | | | BSA |

NC: not calculated

The lower the internalization rate and the faster the receptor recycling rate the more the receptor is retained at the membrane. Table 5 shows receptor internalization data and table 6 shows receptor recycling data. In the latter, a lower T_{1/2} value is indicative of a more rapid return of receptors to the cell surface and an increased number of receptors available to interact with the compound [see e.g. Jones et. al. Nat. Commun. 2018, 9, 1602.]. This may correlate with the tolerability of the described Compounds in an inverse manner, i.e. the less potent the more tolerable.

### Pharmacokinetic Experiments

### Mouse PK:

For all in vivo experiments, Compounds disclosed herein were prepared in a stock solution in PBS where Compounds had a concentration of 1 mg/mL. These stock solutions were then diluted with saline to obtain the concentrations disclosed in the experiments.

For all in vivo experiments, semaglutide was purchased as a clinical formulation called OZEMPIC, which is a stock solution of 1.34 mg/mL semaglutide. The stock solution comprises the inactives disodium phosphate dihydrate, 1.42 mg; propylene glycol, 14.0 mg; phenol, 5.50 mg; and water for injections. OZEMPIC has a pH of approximately 7.4. This stock solution was then diluted with saline to obtain the concentrations disclosed herein.

For all compounds: to obtain pharmacokinetic parameters, three C57BL/6 mice (20-30 weeks of age) fed a high fat diet (60% calories from fat) from 6 weeks of age were dosed subcutaneously (s.c.) with compound in saline at 0.24 mg/kg using a dose volume of 5 mL/kg, wherein the concentration of a compound was 48 μg/mL. Following compound administration, blood samples were collected in EDTA-coated tubes, via tail nick, at 0.5, 1, 3, and 6 h post dose on day 0 and then at 24, 48, 72, 96, 168, 240, 336, and 408 h post dose (i.e., days 1, 2, 3, 4, 7, 10, 14, and 17). Plasma portion was obtained by centrifugation (13,000 rpm, 4° C., for 5 min); and a 30 μL aliquot of mouse plasma was transferred into a 96-well plate for bioanalysis. Calibration standards and QC samples were prepared in blank mouse plasma (plasma of untreated mice). The PK samples were diluted 2 times with blank mouse plasma (10 μL sample plus 10 μL blank mouse plasma) and were extracted using a protein precipitation procedure involving addition of 150 μL methanol containing internal standard. The samples were vortexed and centrifuged at 4000 rpm for 15 min at 4° C. A 125 μL aliquot of supernatant was transferred to a 96-well plate and 100 μL of water was added to each well and vortexed. Samples were analyzed and quantified by LC-MS/MS using the conditions outlined below.

### LC/MS/MS Method

### Mass Spectrometer: Thermo QExactive HFX

### Liquid Chromatograph: Thermo Vanquish

### Autosampler (ALS): Thermo Vanquish

### HPLC Conditions

### LC Column: Waters Acquity UPLC Protein BEH C4, 50×2.1 mm, 1.7 um

### Solvent A: 100:0.1 (v:v) Water:Formic Acid

### Solvent B: 100:0.1 (v:v) Acetonitrile:Formic Acid

Injection volume: 10 μL
Column oven temperature: 40° C.
ALS temperature: 4° C.

TABLE 7
Gradient

| Time (min) | % A | % B | Flow [μL/min] |
|---|---|---|---|
| 0.00 | 70 | 30 | 500 |
| 0.50 | 70 | 30 | 500 |
| 3.50 | 5 | 95 | 500 |
| 4.00 | 5 | 95 | 500 |
| 4.01 | 70 | 30 | 500 |
| 4.50 | 70 | 30 | 500 |

### MS Conditions

### Ion Source: HESI

### Polarity: Positive

Aux gas heater temperature: 380° C.
Sheath gas flow rate: 60
Aux gas flow rate: 14
Sweep gas flow rate: 3
Ion spray voltage: 3500 V
Capillary temperature: 320° C.

TABLE 8
DIO mice PK data: (Stability Assessment)

| | Dose | | Cₘₐₓ | AUC_{inf} | |
|---|---|---|---|---|---|
| compound | (mg/kg) | Tₘₐₓ (day) | (nmol/L) | (day*nmol/L) | T_{1/2} (day) |
| Compound 1 | 0.24 | 1.0 ± 0.0 | 308 ± 58.1 | 854 ± 59.2 | 1.4 ± 0.05 |
| Compound 2 | 0.24 | 1.0 ± 0.0 | 338 ± 54.6 | 1150 ± 115 | 1.5 ± 0.04 |
| Compound 3 | 0.24 | 1.0 ± 0.0 | 261 ± 41.9 | 683 ± 72.5 | 1.0 ± 0.05 |
| Compound 4 | 0.24 | 1.3 ± 0.6 | 241 ± 37.6 | 892 ± 37.4 | 1.7 ± 0.2 |
| Compound 5 | 0.24 | 1.0 ± 0.0 | 548 ± 106 | 1650 ± 150 | 2.1 ± 0.2 |
| Compound 6 | 0.24 | 0.25 ± 0 | 284 ± 48.2 | 371 ± 70 | 0.5 ± 0.01 |
| Compound 7 | 0.24 | 1.0 ± 0.0 | 354 ± 47.1 | 1050 ± 216 | 1.37 ± 0.14 |
| semaglutide | 0.12 | 0.21 ± 0.07 | 219 ± 25.5 | 194 ± 26.3 | 0.35 ± 0.09 |

Cynomolgous Monkey PK:

To obtain pharmacokinetic parameters, obese male cynomolgus monkeys were given a single s.c. dose of compound with formulation concentration of 30, 60 or 90 μg/mL in saline using a dose volume of 0.5 mL/kg. Monkeys were dosed in the morning prior to feeding but were not fasted. The animals were bled via the saphenous vein at defined intervals (pre dose, 0.25, 0.5, 1, 3, 7, 24, 48, 96, 168, 240, 336, and 504 h post dose). Blood was drawn into vacutainer tubes containing K₂EDTA and stored on ice until centrifugation. The plasma portion was obtained by centrifugation for 10 min at 1000-2000 RCF (generally 1300 RCF) at 4° C. 50 μL aliquot of monkey plasma was transferred into a 96-well plate for bioanalysis. Calibration standards and QC samples were prepared in blank cynomolgous obese monkey plasma (plasma of untreated obese monkeys). The PK samples were diluted 2 times with blank obese monkey plasma (10 μL sample plus 10 μL blank obese monkey plasma) and were extracted using a protein precipitation procedure involving addition of 150 μL methanol containing an internal standard. The samples were vortexed and centrifuged at 4000 rpm for 15 min at 4° C. A 125 μL aliquot of supernatant was transferred to a 96-well plate and 100 μL of water was added to each well and vortexed. Samples were analyzed and quantified by LC-MS/MS using the conditions outlined.

TABLE 9
Obese Monkey PK data: (Stability Assessment)

| | Dose | | | AUC_{inf} | |
|---|---|---|---|---|---|
| | (mg/ | Tₘₐₓ | Cₘₐₓ | (day*nmol/ | T_{1/2} |
| compound | kg) | (day) | (nmol/L) | L) | (day) |
| Compound 1 | 0.045 | 1.0 ± 0.0 | 119 ± 29.0 | 1020 ± 202 | 4.8 ± 0.4 |
| Compound 2 | 0.015 | 2.3 ± 1.2 | 28.2 ± 1.7 | 294 ± 26.7 | 6.0 ± 1.1 |
| Compound 3 | 0.015 | 2.3 ± 1.2 | 24.4 ± 8.3 | 196 ± 9.8 | 3.8 ± 0.4 |
| Compound 5 | 0.030 | 1.3 ± 0.6 | 31.4 ± 10.4 | 264 ± 101 | 4.4 ± 0.4 |
| semaglutide | 0.004 | 1.0 ± 0.0 | 7.11 ± 2.2 | 35.5 ± 7.1 | 2.8 ± 0.2 |

Data assessed in Tables 8 and 9 provide evidence for a superior in vivo stability against metabolic degradation of the present Compounds as compared to semaglutide.

### Efficacy Study: Acute Food Intake Study:

Food intake (FI) following a single SC subcutaneous (s.c.) dose of each tested compound with formulation concentration of 24, 38 or 48 μg/ml in saline (dose volume of 5 mL/kg) (e.g., Compound 1) was assessed in diet-induced obese (DIO) male mice (C57BL/6 mice fed a high fat diet (60% calories from fat) from 6 weeks of age). Males 24-30 weeks of age were used in the studies. Animals were housed one per cage in a normal light cycle (6:00 am-6:00 pm lights on, otherwise lights off) room, under an approved IACUC protocol. Mean food intake (FI) (24 h food intake measured over a 3-day period prior to study start) was used as a baseline. At the start of the study, food weight was recorded and animals were subcutaneously dosed with a test compound. Food intake weight was measured 24 h post dosing of a test compound. Food intake (FI) after 24 hours in obese mice was assessed following the subcutaneous administration of a single dose of a compound at the indicated dosage; the resulting data are shown in Table 10. As a comparison, the effect of semaglutide was assessed.

TABLE 10
Food Intake in DIO model mice after single s.c. administration

| | FI @ 24 h | |
|---|---|---|
| compound | (% baseline) | Dose |
| Compound 1 | −88 ± 2 | 240 μg/kg |
| Compound 2 | −91 ± 5 | 240 μg/kg |
| Compound 3 | −97 ± 3 | 240 μg/kg |
| Compound 4 | −54 ± 9 | 240 μg/kg |
| Compound 5 | −72 ± 3 | 120 μg/kg |
| | −81 ± 5 | 190 μg/kg |
| | −89 ± 2 | 240 μg/kg |
| Compound 6 | −67 ± 8 | 240 μg/kg |
| Compound 7 | −49 ± 12 | 240 μg/kg |
| Compound 9 | −44 ± 12 | 240 μg/kg |
| semaglutide | −86 ± 1 | 120 μg/kg |

### Efficacy Studies

Efficacy (food intake and body weight reduction) following treatment of compound(s) (e.g., Compound 1) was assessed in diet-induced obese (DIO) male mice (C57BL/6 mice fed a high fat diet (60% calories from fat) from 6 weeks of age). Males 24-30 weeks of age were used in the studies (n=7/group). Animals were housed one per cage in a normal light cycle room, under an approved IACUC protocol. Mice were assigned to vehicle (saline) or treatment group(s) based on the means of body weights (BW) and food intake (FI) (24 h food intake, measured over a 3-day period prior to study start). At the start of the study, body and food weights were recorded, and animals were subcutaneously dosed with vehicle or compound (12, 24, 29.6, 38, or 48 ug/ml in saline using a dose volume of 5 ml/kg). Compound(s) or vehicle were given QD or when indicated 03D (every 3 days). Semaglutide was dosed QD. Body weight and food intake were measured daily. Doses of Compound 1 and semaglutide were selected based on (i) max efficacy assessed in separate studies and in-line with published data (Semaglutide) and (ii) equimolar concentration. Doses of the stereoisomers (compound 2 and 3) were selected based on maximum efficacy of compound 1 assessed in a separate study.

Body weight loss in obese mice following the subcutaneous administration of a compound after 18, 24, and 30 days is shown in Table 11. Test compounds and vehicle were dosed QD or 03D depending on the dosage; semaglutide was dosed QD; all compounds were dissolved in saline.

TABLE 11
Body weight loss in DIO model mice after s.c. administration

| | Averaged | | Weight loss | |
|---|---|---|---|---|
| compound | Initial BW (g) | Dose | (% baseline) | Day |
| Compound 5 | 53.4 ± 0.4 | 60 μg/kg QD | −7.4 ± 1.4 | 24 |
| | | 120 μg/kg Q3D | −10.7 ± 1.1 | |
| | | 240 μg/kg Q3D | −19.0 ± 1.2 | |
| Compound 1 | 58.2 ± 0.5 | 148 μg/kg Q3D | −17.1 ± 2.2 | 30 |
| | | 240 μg/kg QD | −33.4 ± 3.8 | |
| semaglutide | | 120 μg/kg QD | −17.8 ± 1.2 | |
| Compound 1 | 49.3 ± 0.3 | 190 μg/kg Q3D | −19.5 ± 3.0 | 18 |
| Compound 2 | | | −14.1 ± 1.1 | |
| Compound 3 | | | −25.6 ± 2.9 | |
| Compound 1 | 49.3 ± 0.3 | 240 μg/kg Q3D | −20.5 ± 2.8 | 18 |
| Compound 2 | | 240 μg/kg QD | −20.5 ± 3.0 | |
| Compound 3 | | 240 μg/kg Q3D | −24.2 ± 2.7 | |

### Cyno Efficacy Study:

The pharmacokinetic (PK)/pharmacodynamics (PD) relationship of a novel long-acting GLP1R agonist (Compound 1) was assessed in obese cynomolgus monkeys by evaluating its effect on BW and FI. Efficacy was defined as a reduction in FI and BW. Tolerability, assessed as reduced and/or absence of emesis and retained interest in a selection of fruits and vegetables and peanuts as treats.

Monkeys were acclimated to the study diet (5TUR diet, 1 g pellets (TestDiet Cat #1815639-310)) over the course of at least one week. Following the acclimation period, food intake was recorded, for one week prior to the first day of dosing, to establish baseline food intake (FI). Baseline body weight was calculated as the average of two independent measurements obtained within a seven-day period, prior to the first day of dosing.

Ten male obese cynomolgus monkeys were given a subcutaneous (s.c.) injection of either vehicle (n=4), or compound (n=6) (0.03 mg/kg using a dose volume of 0.5 mL/kg, hence a 0.06 mg/mL concentration). Test compound was dissolved in saline at the indicated concentration. Monkeys were dosed in the morning prior to feeding but were not fasted. Food intake was measured daily throughout the study. Monkeys were given a pre-weighed amount of food, 200 g/day; one-half of their allotment was given in the morning and the remainder in the afternoon. Leftover food was weighed the following morning to determine daily FC. Body weight (BW) was measured twice per week.

TABLE 12
Change in body weight in obese monkeys
Change in Body Weight Over Time

| | Compound 1 | Vehicle (saline) |
|---|---|---|
| Time in Days | 30 μg/kg s.c. Q1W | s.c. Q1W |
| 0 | 0.0% ± 0.0% | 0.0% ± 0.0% |
| 4 | −1.6% ± 1.1% | −0.2% ± 0.3% |
| 7 | −1.2% ± 0.7% | 0.5% ± 0.2% |
| 11 | −1.8% ± 1.2% | 0.6% ± 0.2% |
| 14 | −2.2% ± 1.0% | 0.6% ± 0.5% |
| 18 | −2.7% ± 1.5% | 0.9% ± 0.6% |
| 21 | −2.6% ± 1.4% | 1.0% ± 0.4% |
| 25 | −3.1% ± 1.7% | 1.6% ± 0.8% |
| 28 | −3.1% ± 1.6% | 1.2% ± 0.9% |
| 32 | −3.5% ± 2.1% | 1.7% ± 1.0% |
| 35 | −4.2% ± 1.9% | 1.4% ± 1.0% |
| 39 | −4.2% ± 2.2% | 1.2% ± 0.8% |
| 42 | −3.7% ± 2.2% | 1.8% ± 1.2% |

TABLE 13
Change in food intake in obese monkeys
Relative Change of Food Intake (FI) Over Time

| | Compound 1 | | Vehicle | |
|---|---|---|---|---|
| Time in | 30 μg/kg | | (saline) | |
| Days | s.c. Q1W | +/− SEM | s.c. Q1W | +/− SEM |
| 0 | 0.0% | 0.0% | 0.0% | 0.0% |
| 1 | −35.0% | 16.0% | −2.5% | 4.1% |
| 2 | −52.8% | 18.6% | −5.4% | 6.0% |
| 3 | −50.2% | 17.9% | −12.6% | 7.3% |
| 4 | −38.2% | 15.1% | −0.7% | 8.2% |
| 5 | −27.6% | 12.1% | −4.0% | 2.3% |
| 6 | −20.4% | 10.4% | 1.3% | 2.1% |
| 7 | −10.2% | 4.8% | 8.8% | 8.7% |
| 8 | −31.3% | 14.1% | −1.9% | 6.7% |
| 9 | −40.2% | 17.3% | −3.0% | 8.5% |
| 10 | −39.2% | 15.9% | 0.7% | 0.6% |
| 11 | −29.6% | 13.3% | 5.5% | 5.9% |
| 12 | −26.0% | 13.0% | −6.8% | 7.6% |
| 13 | −23.0% | 10.3% | −2.8% | 2.4% |
| 14 | −23.3% | 11.2% | −5.3% | 2.8% |
| 15 | −29.0% | 14.3% | 8.1% | 7.4% |
| 16 | −42.7% | 18.4% | 3.7% | 5.8% |
| 17 | −39.2% | 16.1% | −15.2% | 21.8% |
| 18 | −35.7% | 15.4% | 6.2% | 5.3% |
| 19 | −25.0% | 14.8% | −0.4% | 4.2% |
| 20 | −24.4% | 12.7% | 2.5% | 14.1% |
| 21 | −25.0% | 14.8% | 7.7% | 6.9% |
| 22 | −35.7% | 16.8% | −0.5% | 0.4% |
| 23 | −49.4% | 16.2% | −0.7% | 5.6% |
| 24 | −37.5% | 16.4% | −8.2% | 11.1% |
| 25 | −32.0% | 15.9% | 12.3% | 11.8% |
| 26 | −28.3% | 14.7% | 1.9% | 2.8% |
| 27 | −19.7% | 16.1% | 3.8% | 14.0% |
| 28 | −25.0% | 14.6% | −4.0% | 6.8% |
| 29 | −28.8% | 15.2% | 9.9% | 9.1% |
| 30 | −42.2% | 18.4% | 3.1% | 4.8% |
| 31 | −42.1% | 18.7% | −19.9% | 10.6% |
| 32 | −32.7% | 15.7% | 9.9% | 9.7% |

Compound 1 suppresses food intake and reduces body weight in obese monkeys (see Tables 12 and 13). All data are expressed as mean±SEM, n=7/group.

### Tolerability Assessment:

Surprisingly, it was found that the compounds described herein were much better tolerated when administered to obese monkeys (assessed against semaglutide as a comparator). While no monkeys showed any signs of vomiting after administration of compounds 1, 2, 5, or 9, and 1/6 monkeys showed vomiting with compound 3, all monkeys receiving semaglutide vomited (see table 14).

TABLE 14
Emesis and FI assessment pursuant to a single
dose s.c. administration of a compound

| | | Single | Emesis (number of monkeys |
|---|---|---|---|
| | FI reduction | dose | vomited/total animals |
| | observed | S.C. | in the study) |
| Semaglutide | Yes | 30 μg/kg | Yes (3/3) |
| Compound 1 | Yes | 30 μg/kg | No (0/2) |
| Compound 2 | Yes | 30 μg/kg | No (0/6) |
| Compound 3 | Yes | 30 μg/kg | Yes (1/6) |
| Compound 5 | Yes | 15 μg/kg | No (0/3) |
| | Yes | 30 μg/kg | No (0/3) |
| Compound 9 | Yes | 30 μg/kg | No (0/3) |

### Chemistry Section

### A: Analytical Section

### LCMS Methods:

### Method A

| Flow | 1 mL/min | | |
|---|---|---|---|
| Eluents | A: Water (0.1% formic acid); | | |
| | B: ACN (0.1% formic acid) | | |
| Gradient | Time | % A | % B |
| | 0.00 | 95 | 40 |
| | 1.40 | 5 | 98 |
| | 2.05 | 5 | 98 |
| | 2.10 | 95 | 40 |

| | |
|---|---|
| Column | Acquity BEH 1.7 μm 2.1 × 50 mm |
| Column | 50° C. |
| Temperature | |
| Mass spectrometer | Single Quadrupole ESI scan range 120-1600 |
| UPLC | Waters Acquity |

### Method B

| Flow | 1.5 mL/min | | |
|---|---|---|---|
| Eluents | A: Water (0.037% TFA); B: ACN (0.018% TFA) | | |
| Gradient | Time | % A | % B |
| | 0.00 | 95 | 5 |
| | 0.80 | 5 | 95 |
| | 1.20 | 5 | 95 |
| | 1.21 | 95 | 5 |
| | 1.55 | 95 | 5 |

| | |
|---|---|
| Column | Kinetex ® 5 μm 30 × 2.1 mm S/N: H17-247175 |
| Column | 50° C. |
| Temperature | |
| Ionization source | ESI |
| Instrument | SHIMADZU LCMS-2020 |
| Detector | PDA (220 nm & 254 nm) |
| Scan range | 100-1000 |

### Method C

| Flow | 1.0 mL/min | | |
|---|---|---|---|
| Stop Time | 5.20 min | | |
| Eluents | A: Water (0.1% TFA); B: ACN (0.1% TFA) | | |
| Gradient | Time | % A | % B |
| | 0.00 | 98 | 2 |
| | 4.40 | 2 | 98 |
| | 5.15 | 2 | 98 |
| | 5.19 | 98 | 2 |

| | |
|---|---|
| Column | AcQuity UPLC BEH C18 1.7 μm 2.1 × 50 mm |
| Column | 50° C. |
| Temperature | |
| UV | 210-400 nm |

### Method D

| Flow | 1.0 mL/min | | |
|---|---|---|---|
| Stop Time | 2.00 min | | |
| Eluents | A: Water (0.1% formic acid); B: ACN (0.1% formic acid) | | |
| Gradient | Time | % A | % B |
| | 0.00 | 98 | 2 |
| | 0.10 | 98 | 2 |
| | 1.50 | 2 | 98 |
| | 1.80 | 2 | 98 |
| | 1.90 | 98 | 2 |
| | 2.00 | 98 | 2 |

| | |
|---|---|
| Column | AcQuity UPLC BEH C18 1.7 μm 2.1 × 30 mm |
| Column | 50° C. |
| Temperature | |
| UV | 210-400 nm |

### Method E

| Flow | 1.0 mL/min | | |
|---|---|---|---|
| Stop Time | 2.20 min | | |
| Eluents | A: Water (0.1% formic acid); B: ACN (0.1% formic acid) | | |
| Gradient | Time | % A | % B |
| | 0.00 | 60 | 40 |
| | 1.40 | 2 | 98 |
| | 2.05 | 2 | 98 |
| | 2.09 | 60 | 40 |

| | |
|---|---|
| Column | AcQuity UPLC BEH C18 1.7 μm 2.1 × 30 mm |
| Column | 50° C. |
| Temperature | |
| UV | 210-400 nm |

### Method F

| Flow | 1.0 mL/min | | |
|---|---|---|---|
| Stop Time | 5.20 min | | |
| Eluents | A: Water (0.1% formic acid); B: ACN (0.1% formic acid) | | |
| Gradient | Time | % A | % B |
| | 0.00 | 98 | 2 |
| | 4.40 | 2 | 98 |
| | 5.15 | 2 | 98 |
| | 5.19 | 98 | 2 |

| | |
|---|---|
| Column | AcQuity UPLC BEH C18 1.7 μm 2.1 × 50 mm |
| Column | 50° C. |
| Temperature | |
| UV | 210-400 nm |

### Method G

| Flow | 1.0 mL/min | | |
|---|---|---|---|
| Stop Time | 5.20 min | | |
| Eluents | A: Water (0.1% formic acid); B: ACN (0.1% formic acid) | | |
| Gradient | Time | % A | % B |
| | 0.00 | 60 | 40 |
| | 3.40 | 2 | 98 |
| | 5.15 | 2 | 98 |
| | 5.19 | 60 | 40 |

| | |
|---|---|
| Column | AcQuity UPLC BEH C18 1.7 μm 2.1 × 50 mm |
| Column | 50° C. |
| Temperature | |
| Temperature | |
| UV | 210-400 nm |
| Mass Range | 100-2050 Da |

### Method H

| Flow | 1.0 mL/min | | |
|---|---|---|---|
| Stop Time | 5.20 min | | |
| Eluents | A: Water (0.1% formic acid); B: ACN (0.1% formic acid) | | |
| Gradient | Time | % A | % B |
| | 0.00 | 60 | 40 |
| | 3.40 | 2 | 98 |
| | 5.15 | 2 | 98 |
| | 5.19 | 60 | 40 |

| | |
|---|---|
| Column | AcQuity UPLC BEH C18 1.7 μm 2.1 × 50 mm |
| Column | 50° C. |
| Temperature | |
| UV | 210-400 nm |

### Method I

| Flow | 1.0 mL/min | | |
|---|---|---|---|
| Stop Time | 5.20 min | | |
| pH | 10.2 | | |
| Eluents | A: Water (5 mM NH₄OH); B: ACN (5 mM NH₄OH) | | |
| Gradient | Time | % A | % B |
| | 0.00 | 60 | 40 |
| | 3.40 | 2 | 98 |
| | 5.15 | 2 | 98 |
| | 5.19 | 60 | 40 |

| | |
|---|---|
| Column | AcQuity UPLC BEH C18 1.7 μm 2.1 × 50 mm |
| Column | 50° C. |
| Temperature | |
| UV | 210-400 nm |

### Method J

| Flow | 1.0 mL/min | | |
|---|---|---|---|
| Stop Time | 2.20 min | | |
| Eluents | A: Water (0.1% formic acid); B: ACN (0.1% formic acid) | | |
| Gradient | Time | % A | % B |
| | 0.00 | 98 | 2 |
| | 0.06 | 98 | 2 |
| | 1.76 | 2 | 98 |
| | 2.00 | 2 | 98 |
| | 2.16 | 98 | 2 |

| | |
|---|---|
| Column | AcQuity UPLC CSH C18 1.7 μm 2.1 × 50 mm |
| Column | 50° C. |
| Temperature | |
| UV | 210-400 nm |
| Mass Range | 100-2050 Da |

### Method K

| Flow | 1.0 mL/min | | |
|---|---|---|---|
| Stop Time | 5.20 min | | |
| Eluents | A: Water (0.1% TFA); B: ACN (0.1% TFA) | | |
| Gradient | Time | % A | % B |
| | 0.00 | 98 | 2 |
| | 0.06 | 98 | 2 |
| | 1.76 | 2 | 98 |
| | 2.00 | 2 | 98 |
| | 2.16 | 98 | 2 |

| | |
|---|---|
| Column | AcQuity UPLC CSH C18 1.7 μm 2.1 × 50 mm |
| Column | 80° C. |
| Temperature | |
| UV | 210-400 nm |

### B: Synthetic Section

### Intermediate 1: Benzyl 11-bromoundecanoate

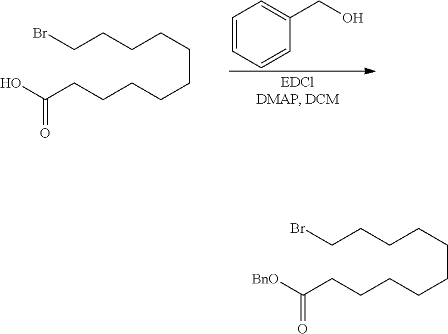

To a mixture of 11-bromoundecanoic acid (4.60 kg, 17.3 mol, 1.1 equiv) in DCM (26.5 kg), was added EDCI (3.8 kg, 20.2 mol, 1.28 equiv) in portions at 0° C. along with DMAP (98 g, 0.8 mol, 0.05 equiv). Benzyl alcohol (1.70 kg, 15.7 mol, 1.00 equiv) was then added dropwise. After stirring at 20° C. for 4 h, water (70.0 kg) was added dropwise. The reaction mixture was then concentrated under vacuum. Heptane (23.2 kg) and 19% NaCl solution (17 kg) were added and the phases separated. The organic phase was washed with 5% Na₂CO₃, 25.0 kg; 19% NaCl solution, 25.0 kg (×2), 5.2% HCl aqueous solution, 25.0 kg; 19% NaCl solution, 25.0 kg, water (5.0 kg), and brine (5.0 kg). The organic phase was then concentrated under vacuum at 50° C. to provide Intermediate 1 which was used as is for next step. ¹H NMR (400 MHz, Chloroform-d) δ ppm 1.18-1.36 (m, 10H) 1.37-1.47 (m, 2H) 1.64 (quin, J=7.33 Hz, 2H) 1.85 (dt, J=14.56, 7.06 Hz, 2H) 2.35 (t, J=7.58 Hz, 2H) 3.40 (t, J=6.88 Hz, 2H) 5.11 (s, 2H) 7.28-7.45 (m, 5H).

### Intermediate 2: 1,11-dibenzyl 11-(tert-butyl) docosane-1,11,11-tricarboxylate

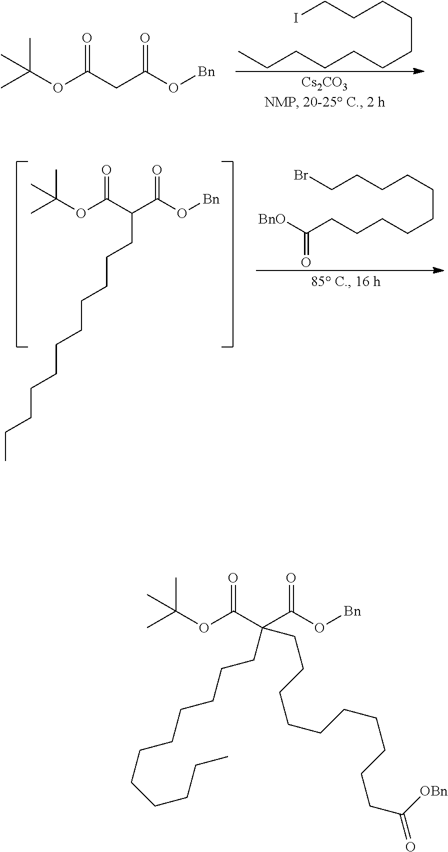

To a solution of benzyl tert-butyl malonate (3.0 kg, 12.0 mol, 1.0 equiv) in NMP (30 L) was added 1-iodoundecane (3.55 kg, 12.58 mol, 1.05 equiv) and Cs₂CO₃ (11.76 kg, 36.09 mol, 3.0 equiv) at 20° C. The resulting mixture was stirred at 20° C. for 6 h and Intermediate 1 (5.53 kg, 15.6 mol, 1.3 equiv) was then added. The reaction mixture was heated up to 85° C. and stirred for 12 h. The mixture was then cooled down to 20° C. and a mixture of water (30 kg) and heptane (10 kg) were added. After stirring for 30 min, the organic phase was separated and washed 3 times with the mixture of brine (5 kg) and MeOH (4 kg). The organic layer was dried over Na₂SO₄, filtered, and concentrated in vacuo. Further purification was accomplished by column chromatography: eluting with heptane/EtOAc=1/0 to 100/1 to provide Intermediate 2. ¹H NMR (400 MHz, Chloroform-d) δ ppm 0.84-0.94 (m, 3H) 1.12 (m, J=6.60 Hz, 4H) 1.19-1.33 (m, 28H) 1.35 (s, 9H) 1.66 (quin, J=7.40 Hz, 2H) 1.85 (t, J=8.44 Hz, 4H) 2.37 (t, J=7.52 Hz, 2H) 5.14 (s, 2H) 5.16 (s, 2H) 7.30-7.42 (m, 10H).

### Intermediate 3: 13-(benzyloxy)-2-((benzyloxy)carbonyl)-13-oxo-2-undecyltridecanoic acid

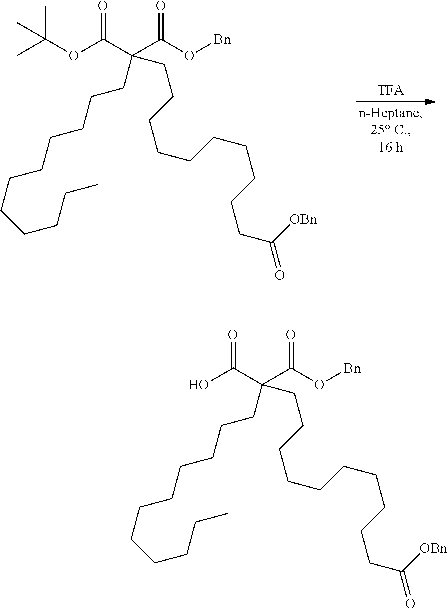 [SMILES: CCCCCCCCCCCC(CCCCCCCCCCC(=O)OCc1ccccc1)(C(=O)OCc1ccccc1)C(=O)OC(C)(C)C ; CCCCCCCCCCCC(CCCCCCCCCCC(=O)OCc1ccccc1)(C(=O)O)C(=O)OCc1ccccc1]

To a solution of Intermediate 2 (6.0 kg, 8.8 mol, 1.0 equiv) in heptane (21 L) was added dropwise TFA (10.0 kg, 88.4 mol, 10.0 equiv) at 20±5° C. After stirring at 20±5° C. for 8 h, most of TFA was removed under reduced pressure and the resulting residue was re-dissolved in heptane (42 L, 7V) and washed with brine (42 L×3). After separation of the phases, the organic phase was concentrated to provide the crude product as a yellow oil. The crude product was purified by column chromatography: eluting with heptane to heptane:EtOAc=10/1 to provide Intermediate 3. ¹H NMR (400 MHz, Chloroform-d) δ ppm 0.87-0.94 (m, 3H) 0.94-1.05 (m, 2H) 1.19 (br. s., 14H) 1.23-1.37 (m, 16H) 1.65 (quin, J=7.40 Hz, 2H) 1.78-1.91 (m, 2H) 1.93-2.05 (m, 2H) 2.37 (t, J=7.52 Hz, 2H) 5.14 (s, 2H) 5.27 (s, 2H) 7.31-7.44 (m, 10H).

The pure enantiomers of the racemic Intermediate 3 were separated via chiral SFC to provide enantiopure Intermediates 3A and 3B which were used to prepare Compounds 2 and 3 respectively. The parameters for obtaining the enantiopure Intermediates 3A and 3B were:

Instrument: Thar 350 preparative SFC (SFC-18)

### Column: ChiralPak AD, 300×50 mm I.D., 10 μm

Mobile phase: A for CO2 and B for Ethanol

### Gradient: B 40%

Flow rate: 200 mL/min
Back pressure: 100 bar
Column temperature: 38° C.

### Wavelength: 210 nm

Cycle time: ˜3.7 min
Peak 1: R enantiomer (3A)
Peak 2: S enantiomer (3B)

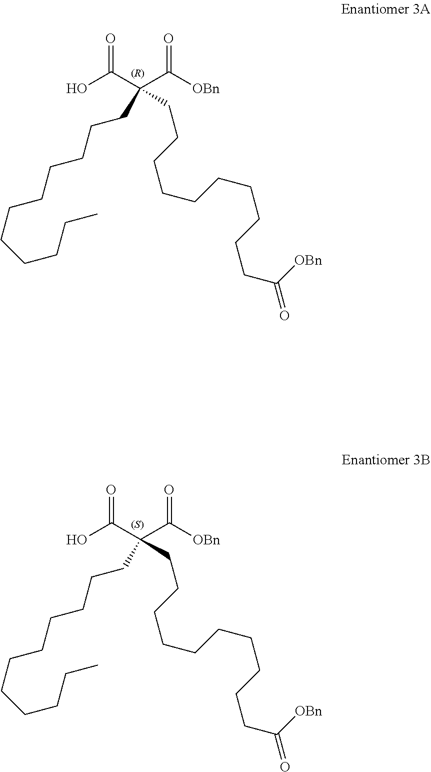

The absolute configuration of the enantiomer 3A was determined by a derivative thereof (shown below); i.e. enantiomer 3A was reacted in a 1st step with oxalylchloride in DMF, followed by reacting the resulting acid chloride with (S)-1-(4-nitrophenyl)-ethan-1-amine, which was then treated with hydrogen in the presence of Pd/C to yield the structure shown below, from which a single X-ray crystal was obtained. Peak 2 of the enantiomeric mixture separated by chiral SFC was therefore associated with the S-configuration and assigned to enantiomer 3B pursuant to the determination of the absolute configuration of enantiomer 3A being R.

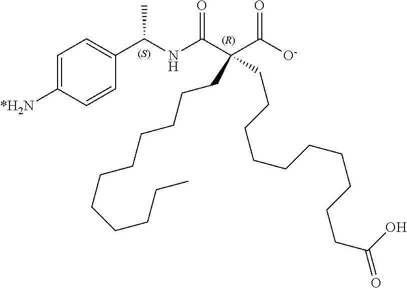

### Derivative of (R)-Enantiomer 3A—Configuration Determined Via X-Ray

### (R)-2-(((S)-1-(4-ammoniophenyl)ethyl)carbamoyl)-2-(10-carboxydecyl)tridecanoate

### Intermediate 4: 14-((benzyloxy)carbonyl)-3,15-dioxo-1-phenyl-14-undecyl-2,19,22,25,28,31,34,37,40,43,46,49,52,55,58,61,64,67,70,73,76,79,82,85,88-pentacosaoxa-16-azahennonacontan-91-oic acid

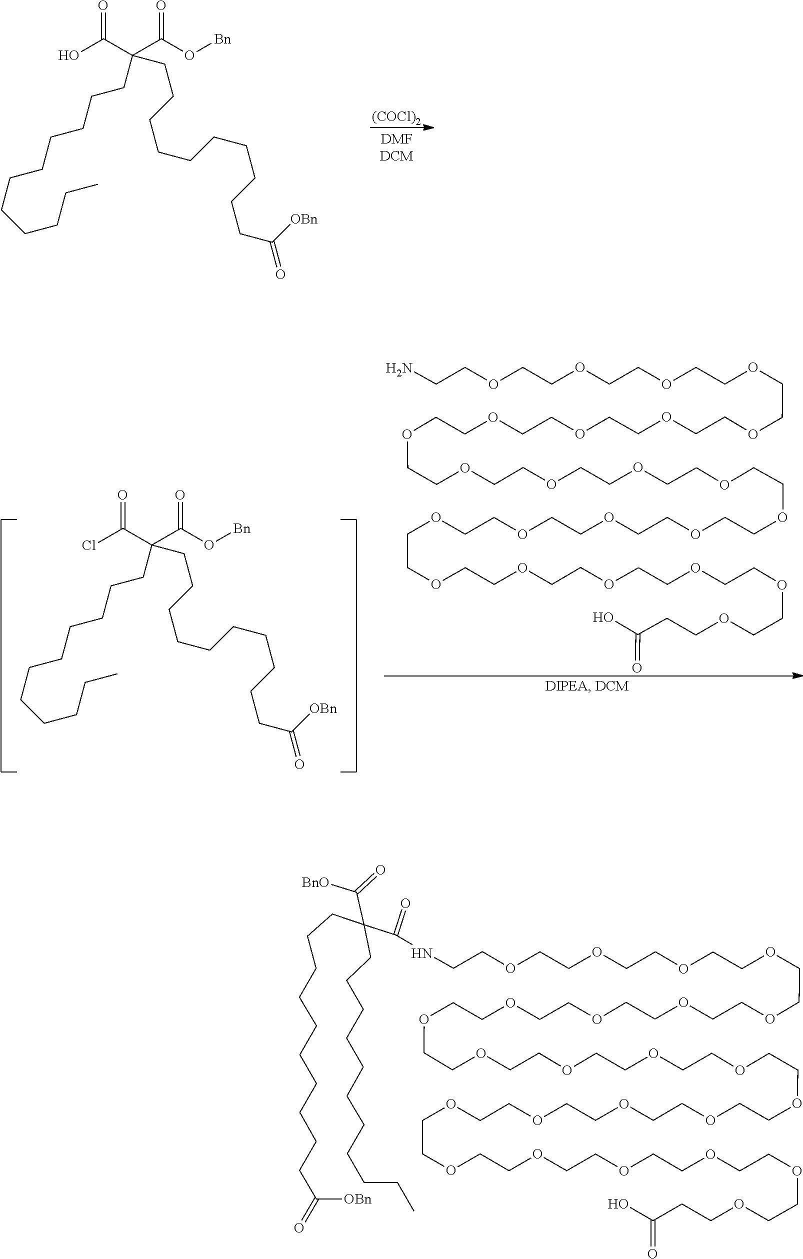

To a flask was added Intermediate 3 (640 g, 1.03 mol), DCM (8.3 kg), and DMF (3 g). The resulting mixture was stirred at 25° C. and oxalyl chloride (170 g, 1.34 mol) was then added dropwise. Stirring was continued for another 2-3 h. Concentration of the reaction mixture and solvent swap with heptane gave a crude mixture to which 8.5 kg DCM was added to form a solution and which was used directly in the following step.

To a flask was added 1-amino-3, 6, 9, 12, 15, 18, 21, 24, 27, 30, 33, 36, 39, 42, 45, 48, 51, 54, 57, 60, 63, 66, 69, 72-tetracosaoxapentaheptacontan-75-oic acid (Amine-PEG24-Acid, 900 g, 0.79 mol), DCM (6.0 kg), and of DIPEA (203 g) and the resulting mixture was stirred at 25° C. The acyl chloride crude solution from step 1 was then added dropwise. The reaction mixture was stirred for another 1-2 h. Acidic resin (1.3 kg) was added and stirring was continued for 30 min. The mixture was then filtered. MgSO₄ (1.3 kg) was added and stirring was continued for 30 min. The mixture was filtered and concentrated to provide a crude residue. The crude residue was purified with Al₂O₃ with mobile phase including MTBE, DCM, MeOH. Then all of the desired fractions were collected and concentrated to provide Intermediate 4. H NMR (400 MHz, Chloroform-d) δ ppm 0.86-0.93 (m, 3H) 0.93-1.04 (m, 2H) 1.19 (br. s., 15H) 1.23-1.37 (m, 15H) 1.61-1.68 (m, 2H) 1.78 (td, J=12.44, 4.34 Hz, 2H) 1.92-2.05 (m, 2H) 2.37 (t, J=7.58 Hz, 2H) 2.62 (t, J=6.05 Hz, 2H) 3.49 (dd, J=6.72, 2.32 Hz, 2H) 3.52-3.59 (m, 2H) 3.59-3.73 (m, 92H) 3.80 (t, J=6.05 Hz, 2H) 5.13 (s, 2H) 5.18 (s, 2H) 7.31-7.42 (m, 10H) 8.09 (t, J=5.26 Hz, 1H).

### Intermediate 5: 77,87-dibenzyl 1-(2,5-dioxopyrrolidin-1-yl) 76-oxo-77-undecyl-3,6,9,12,15,18,21,24,27,30,33,36,39,42,45,48,51,54,57,60,63,66,69,72-tetracosaoxa-75-azaheptaoctacontane-1,77,87-tricarboxylate

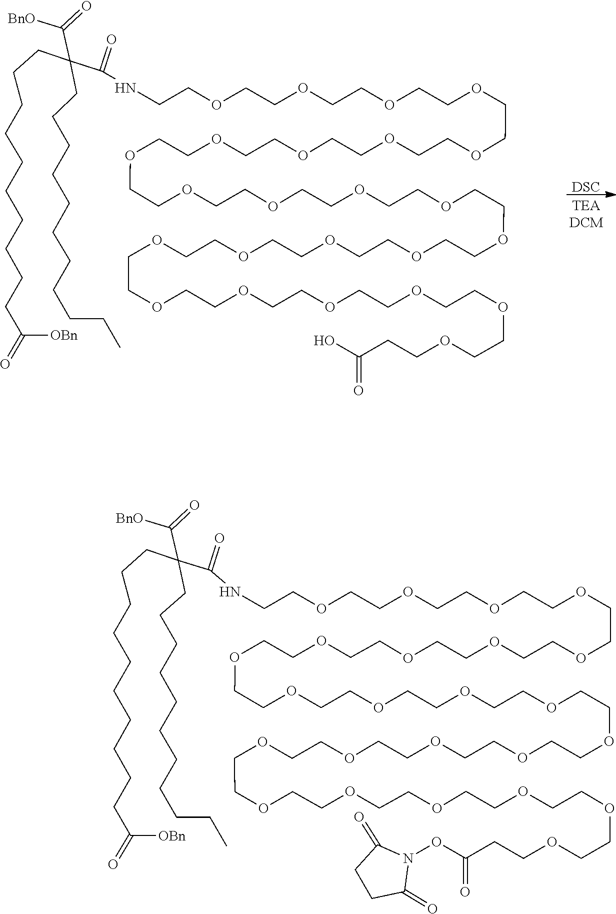 [SMILES: CCCCCCCCCCCC(CCCCCCCCCCC(=O)OCc1ccccc1)(C(=O)NCCOCCOCCOCCOCCOCCOCCOCCOCCOCCOCCOCCOCCOCCOCCOCCOCCOCCOCCOCCOCCOCCOCCOCCOCCC(=O)O)C(=O)OCc1ccccc1 ; "DSC" ; CCCCCCCCCCCC(CCCCCCCCCCC(=O)OCc1ccccc1)(C(=O)NCCOCCOCCOCCOCCOCCOCCOCCOCCOCCOCCOCCOCCOCCOCCOCCOCCOCCOCCOCCOCCOCCOCCOCCOCCC(=O)ON1C(=O)CCC1=O)C(=O)OCc1ccccc1]

To the solution of Intermediate 4 (920 g, 0.53 mol) in DCM (6.1 kg) was added TEA (11 g) and the resulting mixture was stirred to provide a clear solution. DSC (161 g, 0.63 mol) was then added and stirring was continued at 25° C. for 2 h. Acidic resin (180 g) was added and this mixture was stirred for 30 min. MgSO₄ (180 g) was added and stirring was continued for 30 min. The mixture was then filtered to provide a clear light yellow solution. Concentration under vacuum gave crude Intermediate 5, which was used directly in the next step. LCMS Method A: Rt=1.5 min, [M+H₃O+H]⁺²=933.9.

### Intermediate 6: 2-((75-((2,5-dioxopyrrolidin-1-yl)oxy)-75-oxo-3,6,9,12,15,18,21,24,27,30,33,36,39,42,45,48,51,54,57,60,63,66,69,72-tetracosaoxapentaheptacontyl)carbamoyl)-2-undecyltridecanedioic acid

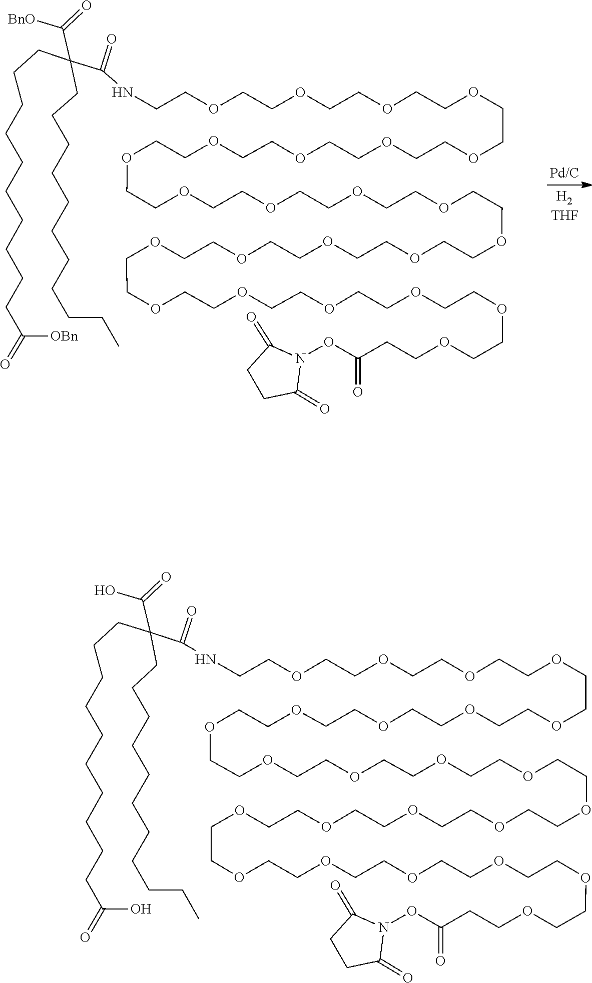 [SMILES: CCCCCCCCCCCC(CCCCCCCCCCC(=O)OCc1ccccc1)(C(=O)NCCOCCOCCOCCOCCOCCOCCOCCOCCOCCOCCOCCOCCOCCOCCOCCOCCOCCOCCOCCOCCOCCOCCOCCOCCC(=O)ON1C(=O)CCC1=O)C(=O)OCc1ccccc1 ; CCCCCCCCCCCC(CCCCCCCCCCC(=O)O)(C(=O)O)C(=O)NCCOCCOCCOCCOCCOCCOCCOCCOCCOCCOCCOCCOCCOCCOCCOCCOCCOCCOCCOCCOCCOCCOCCOCCOCCC(=O)ON1C(=O)CCC1=O]

To a hydrogenation reactor was added Intermediate 5 (986 g, 0.48 mol, 90% purity), THF (7.6 kg), and 10% Pd/C (110 g) followed by MgSO₄ (110 g) and the resulting mixture was purged with N₂ and then with H₂ and stirred at 25° C. for 3-24 h. After complete consumption of starting material, more MgSO₄ (220 g) was added and stirring was continued for an additional 30 min. The reaction mixture was filtered. The cake was washed with 100 mL THF and the filtrate was combined and concentrated to provide Intermediate 6. ¹H NMR (400 MHz, Chloroform-d) δ ppm 0.84-0.94 (m, 3H) 1.17 (br. s., 2H) 1.21-1.39 (m, 30H) 1.57-1.68 (m, 2H) 1.69-1.80 (m, 2H) 1.97-2.10 (m, 2H) 2.34 (t, J=7.21 Hz, 2H) 2.86 (s, 4H) 2.92 (t, J=6.48 Hz, 2H) 3.51-3.73 (m, 96H) 3.87 (t, J=6.48 Hz, 2H) 7.45 (t, J=4.46 Hz, 1H).

### Intermediate 7: 1-benzyl 3-(tert-butyl) 2-undecylmalonate

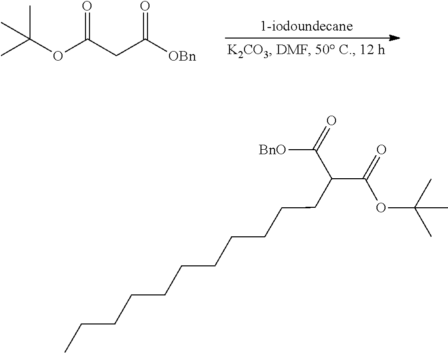 [SMILES: CC(C)(C)OC(=O)CC(=O)OCc1ccccc1 ; CCCCCCCCCCCC(C(=O)OCc1ccccc1)C(=O)OC(C)(C)C]

Benzyl tert-butyl malonate (110 g, 439 mmol, 1 equiv) was taken in DMF (800 mL). To the resulting mixture was added 1-iodoundecane (130 g, 461 mmol, 1.05 equiv) and K₂CO₃ (151 g, 1.10 mol, 2.5 equiv). The resulting suspension was stirred at 50° C. for 12 h. The reaction mixture was then diluted with Ethyl acetate (500 mL), then poured into ice water. The combined organic phases were washed with brine (150 mL) twice, dried with sodium sulfate, filtered, and concentrated under reduced pressure to provide a crude residue. The crude residue was purified by column chromatography (SiO₂, eluting with Petroleum ether/Ethyl acetate=1/0 to 0/1) to provide Intermediate 7 as a colorless oil. ¹H NMR (400 MHz, DMSO-d₆) δ ppm 7.46-7.26 (m, 5H), 5.27-4.98 (m, 2H), 3.44-3.26 (m, 1H), 1.72 (br d, J=6.8 Hz, 2H), 1.33 (s, 9H), 1.28-1.10 (m, 18H), 0.93-0.75 (m, 3H).

### Intermediate 8: 1-benzyl 3-(tert-butyl) 2-allyl-2-undecylmalonate

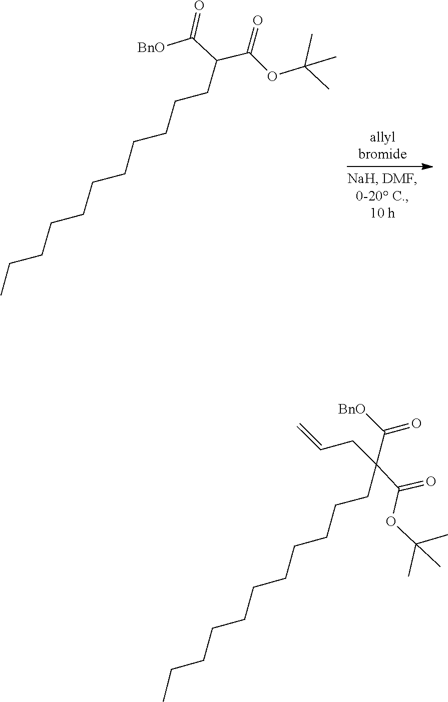

NaH (14.5 g, 364 mmol, 60%, 1.2 equiv) was added dropwise to DMF (1230 mL) at 0° C., and then Intermediate 7 (123 g, 304 mmol, 1 equiv) in DMF (123 mL) was added slowly. The resulting mixture was stirred at 0° C. for 0.5 h, and then the allyl bromide (40.4 g, 334 mmol, 29 mL, 1.1 equiv) was added dropwise. The reaction mixture was stirred at 20° C. for 9.5 h and then diluted with ethyl acetate (2500 mL). The mixture was poured into ice cold saturated NH₄Cl₄ (1200 mL). The organic phase was separated from the aqueous phase, and the aqueous phase was washed twice with ethyl acetate. The organic phases were then combined, washed three times with brine (500 mL), dried over sodium sulfate, filtered, and concentrated under reduced pressure to provide a residue. This residue was purified by column chromatography (SiO₂, eluting with Petroleum ether/Ethyl acetate=1/0 to 98/2) to provide Intermediate 8 as a yellow oil. ¹H NMR (400 MHz, DMSO-d₆) δ ppm 7.50-7.26 (m, 5H), 5.70-5.50 (m, 1H), 5.24-5.01 (m, 4H), 4.99-4.74 (m, 1H), 1.70 (br s, 2H), 1.45-1.34 (m, 3H), 1.27 (s, 9H), 1.25-1.15 (m, 18H), 0.85 (br t, J=6.8 Hz, 3H).

### Intermediate 8A and 8B: 1-benzyl 3-(tert-butyl) 2-allyl-2-undecylmalonate

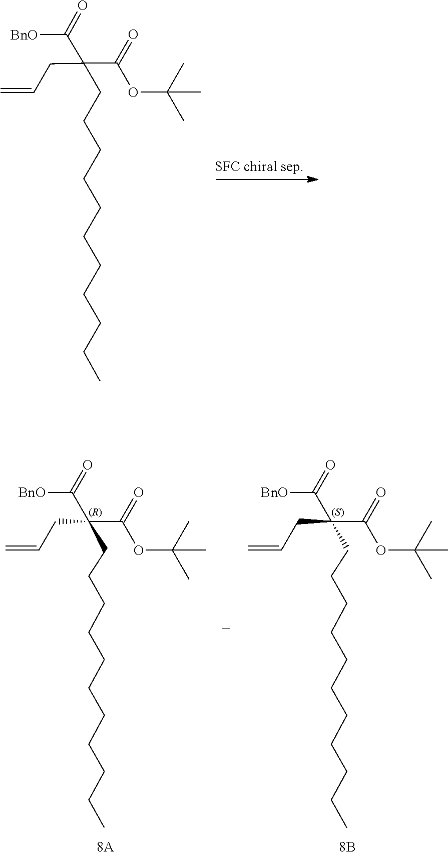

Intermediate 8 (159 g) was purified by SFC separation (Column: Chiralpak IG-3 100*4.6 mm I.D., 3 um; Mobile phase: A: CO2 B: isopropanol (0.05% DEA); Gradient: from 5% to 40% of B in 5 min and hold 40% for 2.5 min, then 5% of B for 2.5 min; Flowrate: 2.5 mL/min; Column temp.: 35° C. ABPR: 1500 psi) to provide Intermediate 8A (peak 1) and Intermediate 8B (peak 2). LCMS Method B: Rt=1.334 min, MS (ESI) m/z [M+Na]⁺=467.3. SFC: Intermediate 8A (peak 1), pure enantiomer (R); SFC: Intermediate 8B (peak 2), pure enantiomer (S).

### Intermediate 9: Benzyl dec-9-enoate

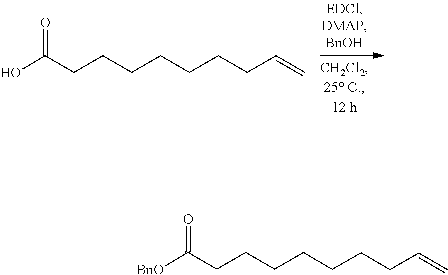 [SMILES: C=CCCCCCCCC(=O)O ; C=CCCCCCCCC(=O)OCc1ccccc1]

To a solution of dec-9-enoic acid (70.0 g, 411 mmol, 76.0 mL, 1 equiv) in DCM (1400 mL) was added BnOH (66.6 g, 616 mmol, 64.1 mL, 1.5 equiv), DMAP (5.02 g, 41.12 mmol, 0.1 equiv), EDCI (94.5 g, 493.3 mmol, 1.2 equiv) and DIEA (63.7 g, 493 mmol, 85.9 mL, 1.2 equiv). The resulting mixture was stirred at 25° C. for 12 h. The reaction mixture was then diluted with DCM (500 mL). The resulting solution was washed with brine (500 mL) twice, dried over Na₂SO₄, filtered, and concentrated under reduced pressure to provide a crude residue. This crude residue was purified by column chromatography (SiO₂, eluting with Petroleum ether/Ethyl acetate=1/0 to 0/1) to provide Intermediate 9 as a colorless oil. ¹H NMR (400 MHz, DMSO-d₆) δ ppm 7.46-7.29 (m, 5H), 5.78 (dd, J=10.4, 16.8 Hz, 1H), 5.13-5.05 (m, 2H), 5.03-4.77 (m, 2H), 2.38-2.26 (m, 2H), 2.13-1.92 (m, 2H), 1.62-1.48 (m, 2H), 1.38-1.28 (m, 2H), 1.28-1.15 (m, 6H).

### Intermediate 10A: 1,11-dibenzyl 11-(tert-butyl) (R) docos-8-ene-1,11,11-tricarboxylate

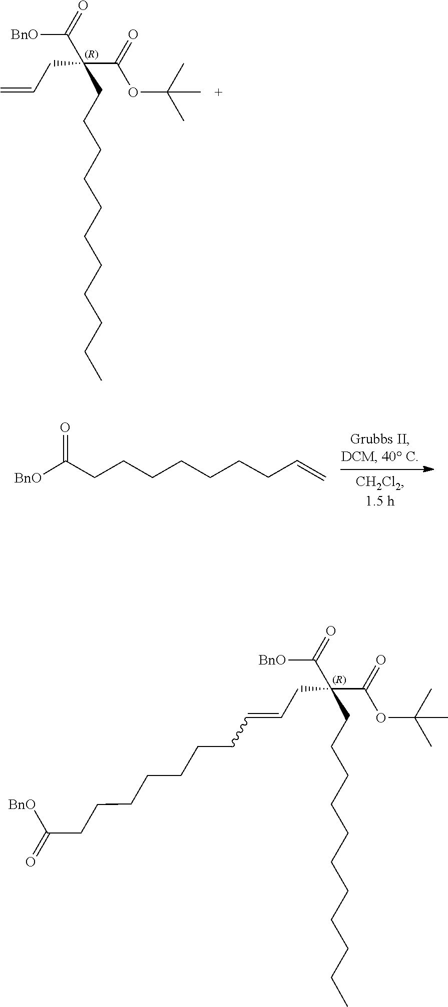

Intermediate 8A (26.0 g, 58.4 mmol, 1 equiv) and Intermediate 9 (30.4 g, 116 mmol, 2 equiv) were dissolved in CH₂Cl₂ (520 mL). Grubbs II (2.38 g, 3.80 mmol, 0.065 equiv) was then added and the resulting mixture was stirred at 40° C. for 1.5 h. The reaction mixture was then concentrated to provide a crude residue. This residue was purified by column chromatography (SiO₂, eluting with Petroleum ether/Ethyl acetate=1/0 to 0/1) to provide Intermediate 10A as a colorless oil. LCMS Method B: Rt=1.456 min, [M+Na]⁺=699.6.

### Intermediate 11A: (S)-13-(benzyloxy)-2-((benzyloxy)carbonyl)-13-oxo-2-undecyltridec-4-enoic acid

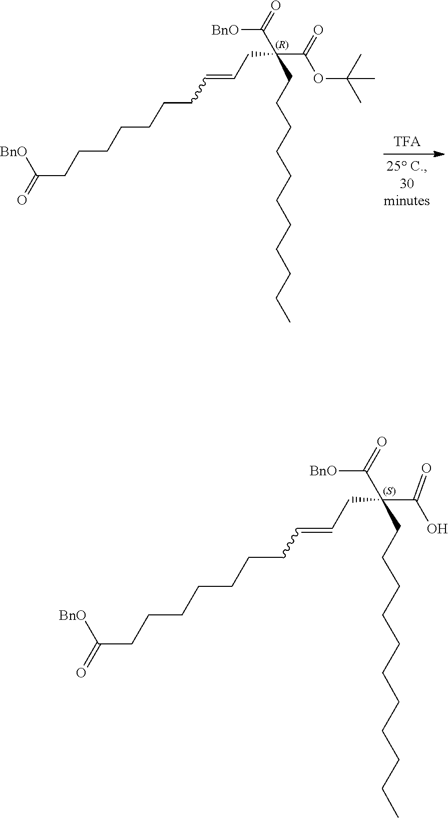

Intermediate 10A (50.0 g, 73.8 mmol, 1 equiv) was dissolved in TFA (500 mL), and the resulting mixture was stirred at 25° C. for 30 min. The reaction mixture was then concentrated to provide a crude residue, which was dissolved in ethyl acetate (500 mL), and then washed by saturated NaHCO₃ (500 mL) twice and brine (100 mL), dried over Na₂SO₄, filtered and concentrated to provide a crude residue. The residue was purified by column chromatography (SiO₂, eluting with Petroleum ether/Ethyl acetate=1/0 to 0/1) to provide Intermediate 11A as a colorless oil. ¹H NMR (400 MHz, DMSO-d₆) δ ppm 7.44-7.14 (m, 10H), 5.41 (br s, 1H), 5.22-4.86 (m, 5H), 2.49-2.41 (m, 2H), 2.37-2.28 (m, 2H), 1.95-1.83 (m, 2H), 1.76-1.65 (m, 2H), 1.58-1.46 (m, 2H), 1.34-0.98 (m, 25H), 0.90-0.76 (m, 3H). LCMS Method B: Rt=1.323 min, [M+H]⁺=622.3.

### Intermediate 12A: 1,11-dibenzyl 11-(2,5-dioxopyrrolidin-1-yl) (R)-docos-8-ene-1,11,11-tricarboxylate

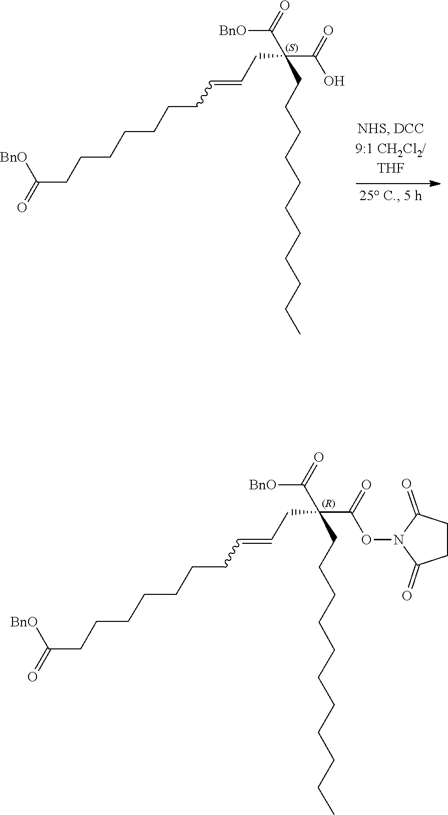

To a solution of Intermediate 11A (29.0 g, 46.7 mmol, 1 equiv) in CH₂Cl₂ (260 mL) and THF (29 mL), was added NHS (5.64 g, 49.0 mmol, 1.05 equiv) and DCC (11.5 g, 56.0 mmol, 11.3 mL, 1.2 equiv) at 25° C. and the resulting mixture was stirred at 25° C. for 5 h. The reaction mixture was then filtered and washed with CH₂Cl₂ (30 mL) thrice. The organic phase was concentrated to provide a crude residue. The crude residue was purified by column chromatography (SiO₂, eluting with Petroleum ether/Ethyl acetate=1/0 to 85/15) to provide Intermediate 12A as light yellow oil. ¹H NMR (400 MHz, Chloroform-d) δ ppm 7.37-7.20 (m, 10H), 5.53-5.34 (m, 1H), 5.24-5.16 (m, 1H), 5.15-5.12 (m, 2H), 5.16-5.12 (m, 2H), 5.07-5.00 (m, 2H), 2.73 (br s, 4H), 2.66-2.53 (m, 2H), 2.34-2.16 (m, 2H), 1.97-1.77 (m, 4H), 1.70-1.47 (m, 3H), 1.37-0.99 (m, 26H), 0.88-0.65 (m, 3H). LCMS Method B: Rt=1.348 min. [M+H]⁺=718.6.

### Intermediate 13A: (S)-14-((benzyloxy)carbonyl)-3,15-dioxo-1-phenyl-14-undecyl-2, 19, 22, 25, 28, 31, 34, 37, 40, 43, 46, 49, 52, 55, 58, 61, 64, 67, 70, 73, 76, 79, 82, 85, 88-pentacosaoxa-16-azahennonacont-11-en-91-oic acid

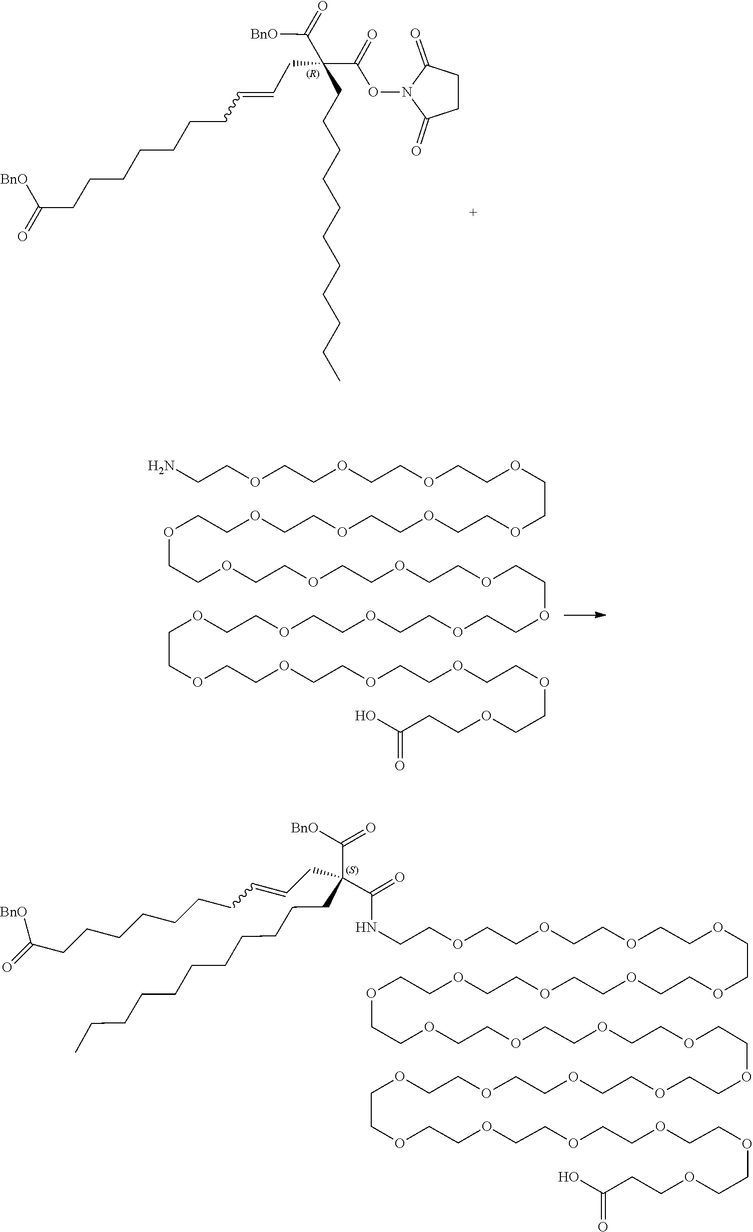

To Intermediate 12A (545 mg, 0.759 mmol) in DMF (3 mL) was added 1-amino-3, 6, 9, 12, 15, 18, 21, 24, 27, 30, 33, 36, 39, 42, 45, 48, 51, 54, 57, 60, 63, 66, 69, 72-tetracosaoxapentaheptacontan-75-oic acid (Amine-PEG24-Acid, 1131 mg, 0.987 mmol), and DIPEA (0.199 mL, 1.139 mmol). After 16 h, the reaction was complete. Volatiles were removed and the resulting residue was purified directly on RPLC (ISCO C18 Gold 150 g column, eluting with 10-100% ACN:water gradient with 0.1% TFA). Fractions containing product were combined, frozen, and lyophilized to provide Intermediate 13A as a thick oil. LCMS Method H: Rt=2.93 min. [M+H]⁺=1750.5.

### Intermediate 14A: 77,87-dibenzyl 1-(2,5-dioxopyrrolidin-1-yl)-(S)-76-oxo-77-undecyl-3, 6, 9, 12, 15, 18, 21,24, 27, 30, 33, 36, 39, 42, 45, 48, 51, 54, 57, 60, 63, 66, 69, 72-tetracosaoxa-75-azaheptaoctacont-79-ene-1,77,87-tricarboxylate

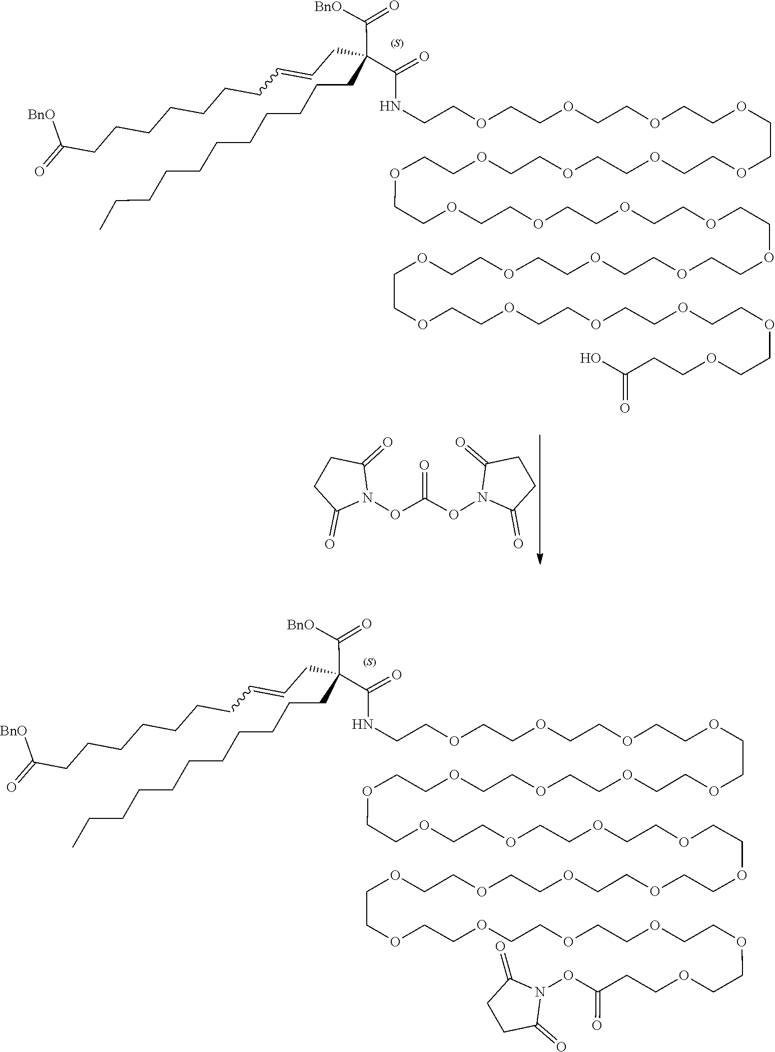

To Intermediate 13A (183 mg, 0.105 mmol) dissolved in 5 mL anhydrous DCM was added DSC (32.2 mg, 0.126 mmol) and DIPEA (0.027 mL, 0.157 mmol) and the resulting mixture was stirred for 16 h, after which the reaction was complete. The crude mixture was injected directly onto a DCM equilibrated ISCO Gold 40 gram column and purified by NPLC (eluting with 0-30% MeOH in DCM, silica). Fractions containing product were combined and concentrated to provide Intermediate 14A as a thick clear oil. LCMS Method H: Rt=2.79 min, [M+H]⁺=1847.5.

### Intermediate 15A: (S)-2-((75-((2,5-dioxopyrrolidin-1-yl)oxy)-75-oxo-3, 6, 9, 12, 15, 18, 21, 24, 27, 30, 33, 36, 39, 42, 45, 48, 51, 54, 57, 60, 63, 66, 69,72-tetracosaoxapentaheptacontyl)carbamoyl)-2-undecyltridecanedioic acid

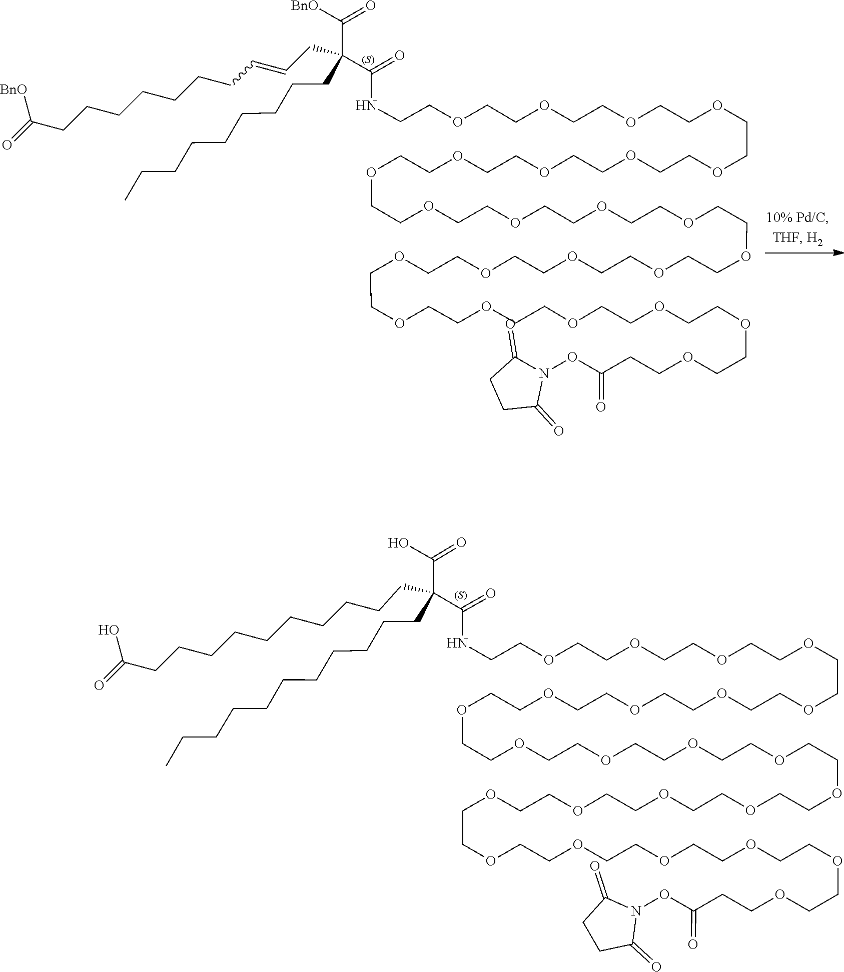

Intermediate 14A (165 mg, 0.089 mmol) was dissolved into 2 mL of anhydrous THF and the atmosphere evacuated and replaced three times with nitrogen. To this mixture was added 10% palladium on carbon (9.51 mg, 8.94 μmol) and the atmosphere was evacuated and replaced with hydrogen from a balloon with magnetic stirring. After 16 h, the reaction was complete. The reaction mixture was filtered through Celite® after dilution with 5 mL anhydrous DCM. The palladium on carbon and Pad were washed with 5 mL DCM twice and all organic phases were combined and concentrated to provide Intermediate 15A. LCMS Method F: Rt=3.29 min. [M+H]⁺=1669.5.

### Intermediate 10B: 1,11-dibenzyl 11-(tert-butyl) (S)-docos-8-ene-1,11,11-tricarboxylate

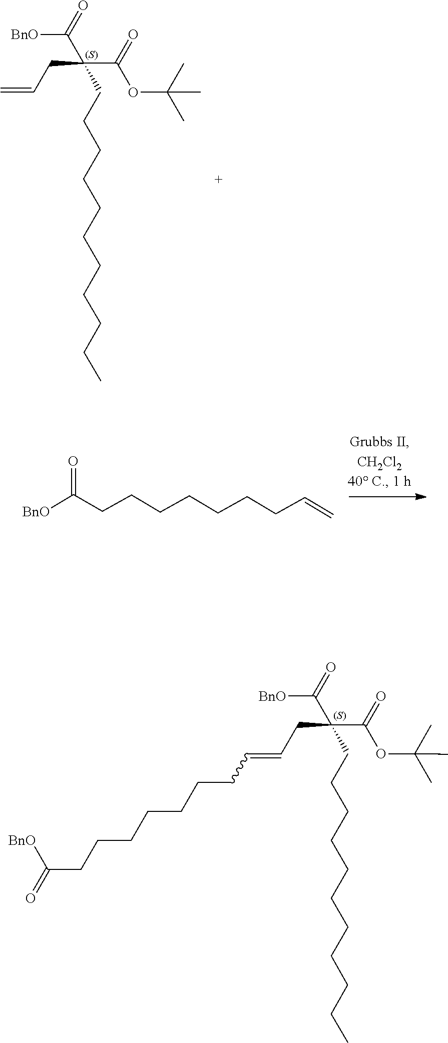

Intermediate 8B (36.0 g, 80.9 mmol, 1 equiv) and Intermediate 3 (42.1 g, 161 mmol, 2 equiv) were dissolved in CH₂Cl₂ (720 mL) and Grubbs II (3.30 g, 5.26 mmol, 0.065 equiv) was then added. The resulting mixture was stirred at 40° C. for 1 h and then concentrated in vacuo to provide a crude residue. The crude residue was purified by column chromatography (SiO₂, eluting with Petroleum ether/Ethyl acetate=1/0 to 0/1) to provide Intermediate 10B as a colorless oil. ¹H NMR (400 MHz, Chloroform-d) δ ppm 7.45-7.25 (m, 10H), 5.50-5.30 (m, 1H), 5.20-5.02 (m, 5H), 2.48-2.40 (m, 1H), 2.39-2.23 (m, 3H), 1.96-1.83 (m, 3H), 1.79-1.64 (m, 1H), 1.61-1.45 (m, 3H), 1.40-1.14 (m, 22H), 1.13-0.94 (m, 3H), 1.14-0.93 (m, 3H), 0.87-0.80 (m, 1H). LCMS Method B: RT=1.448 min. [M−56+H]⁺=622.3.

### Intermediate 11B: (R)-13-(benzyloxy)-2-((benzyloxy)carbonyl)-13-oxo-2-undecyltridec-4-enoic acid

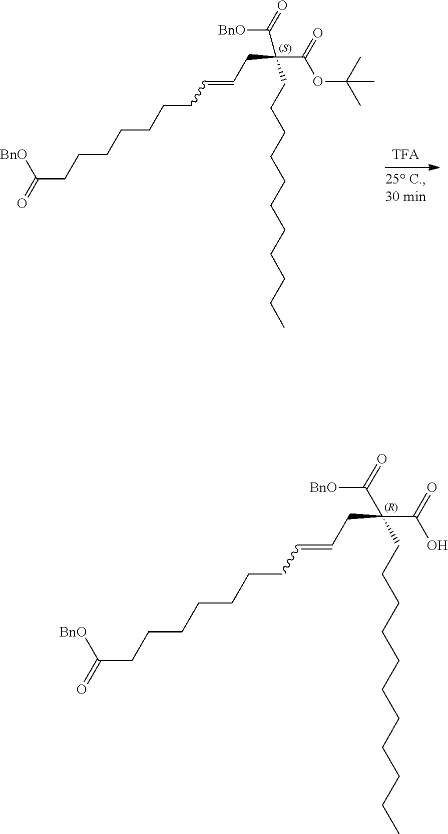

Intermediate 10B (62 g, 91.59 mmol, 1 equiv) was dissolved in TFA (620 mL) and the resulting mixture was stirred at 25° C. for 30 min. The reaction mixture was then concentrated in vacuo to provide a crude residue. The crude residue was dissolved in ethyl acetate (800 mL) and then washed by sat. NaHCO₃ (200 mL) twice and brine (100 mL), dried using Na₂SO₄, filtered, and concentrated to provide a crude residue. The crude residue was purified by column chromatography (SiO₂, eluting with Petroleum ether/Ethyl acetate=1/0 to 0/1) to provide Intermediate 11B as a yellow oil. ¹H NMR (400 MHz, DMSO-d₆) δ 13.15-12.55 (m, 1H), 7.54-6.92 (m, 10H), 5.54-5.33 (m, 1H), 5.25-4.94 (m, 5H), 2.47 (br d, J=7.2 Hz, 1H), 2.33 (br t, J=7.3 Hz, 2H), 1.97-1.83 (m, 2H), 1.79-1.63 (m, 2H), 1.60-1.45 (m, 2H), 1.38-0.96 (m, 26H), 0.92-0.76 (m, 3H). LCMS Method B: RT=1.323 min, MS (ESI) m/z [M+H]⁺=621.6.

### Intermediate 12B: 1,11-dibenzyl 11-(2,5-dioxopyrrolidin-1-yl) (S)-docos-8-ene-1,11,11-tricarboxylate

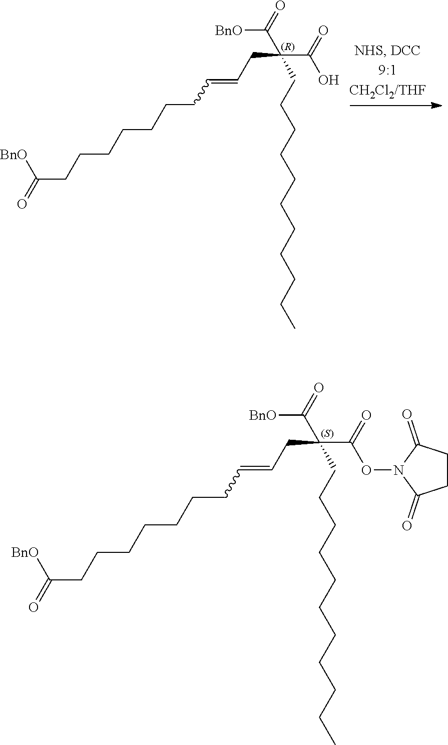

To a solution of Intermediate 11B (27 g, 43.4 mmol, 1 equiv) in CH₂Cl₂ (243 mL) and THF (27 mL) was added NHS (5.26 g, 45.6 mmol, 1.05 equiv) and DCC (10.7 g, 52.1 mmol, 10.5 mL, 1.2 equiv) and the resulting mixture was stirred at 25° C. for 6 h. The reaction mixture was then filtered and washed by CH₂Cl₂ (30 mL) thrice to provide the filtrate which was then concentrated to provide a crude residue. The crude residue was purified by column chromatography (SiO₂, eluting with Petroleum ether/Ethyl acetate=1/0 to 85/15) to provide Intermediate 12B as a light yellow oil. ¹H NMR (400 MHz, Chloroform-d) δ 13.15-12.55 (m, 1H), 7.54-6.92 (m, 10H), 5.54-5.33 (m, 1H), 5.25-4.94 (m, 5H), 2.47 (br d, J=7.2 Hz, 1H), 2.33 (br t, J=7.3 Hz, 2H), 1.97-1.83 (m, 2H), 1.79-1.63 (m, 2H), 1.60-1.45 (m, 2H), 1.38-0.96 (m, 26H), 0.92-0.76 (m, 3H). LCMS Method B: Rt=1.348 min, [M+H]⁺=718.5.

### Intermediate 13B: (R)-14-((Benzyloxy)carbonyl)-3,15-dioxo-1-phenyl-14-undecyl-2,19,22,25,28,31,34,37,40,43,46,49,52,55,58,61,64,67,70,73,76,79,82,85,88-pentacosaoxa-16-azahennonacont-11-en-91-oic acid

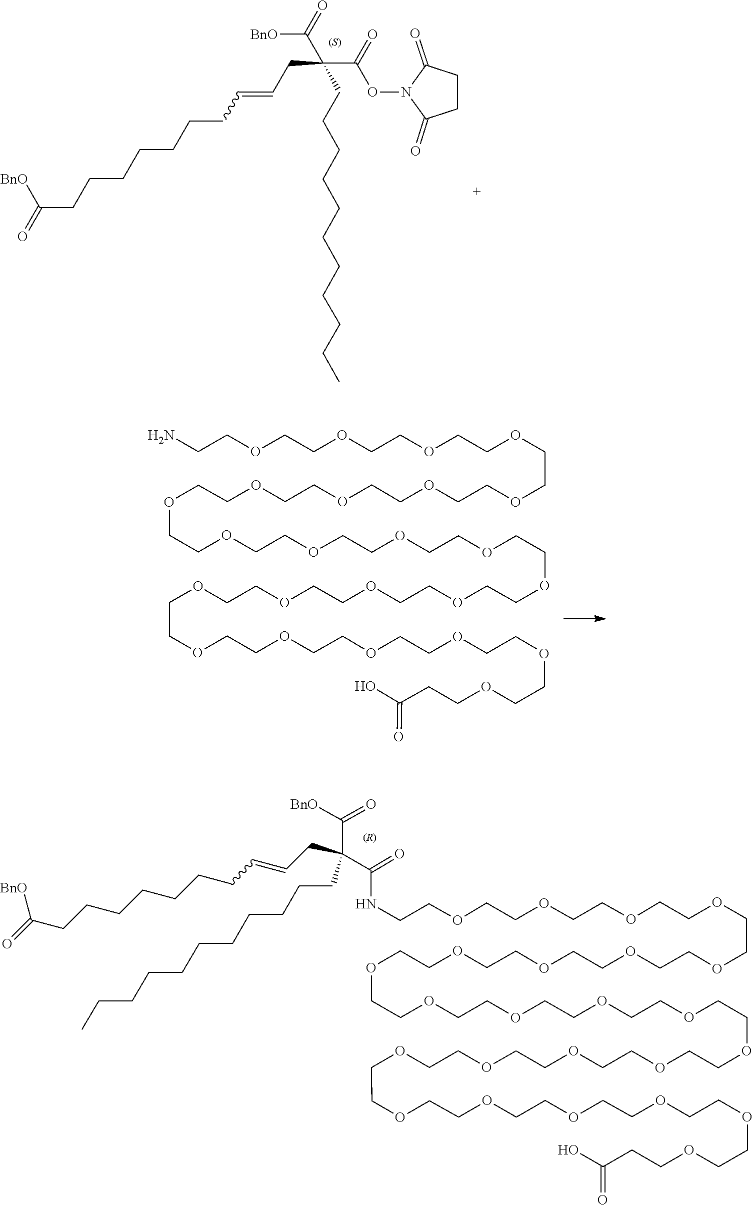

Intermediate 12B (1.07 g, 1.49 mmol) was treated with 1-amino-3,6,9,12,15,18,21,24,27,30,33,36,39,42,45,48,51,54,57,60,63,66,69,72-tetracosaoxapentaheptacontan-75-oic acid (Biopharm, 1.88 g, 1.64 mmol), DIPEA (390 μL, 2.236 mmol) and DMAP (18 mg, 0.05 mmol). After 16 h, the reaction was complete. Volatiles were removed and the resulting residue was purified by RPLC (ISCO 018 Gold 150 g column, eluting with 10-100% ACN:water gradient with 0.1% TFA). Fractions containing product were combined, frozen and lyophilized to provide Intermediate 13B as a thick oil. LCMS Method C: Rt=4.04 min [M+2H]²⁺=875.8.

### Intermediate 14B: 77,87-Dibenzyl 1-(2,5-dioxopyrrolidin-1-yl)-(R)-76-oxo-77-undecyl-3,6,9,12,15,18,21,24,27,30,33,36,39,42,45,48,51,54,57,60,63,66,69,72-tetracosaoxa-75-azaheptaoctacont-79-ene-1,77,87-tricarboxylate

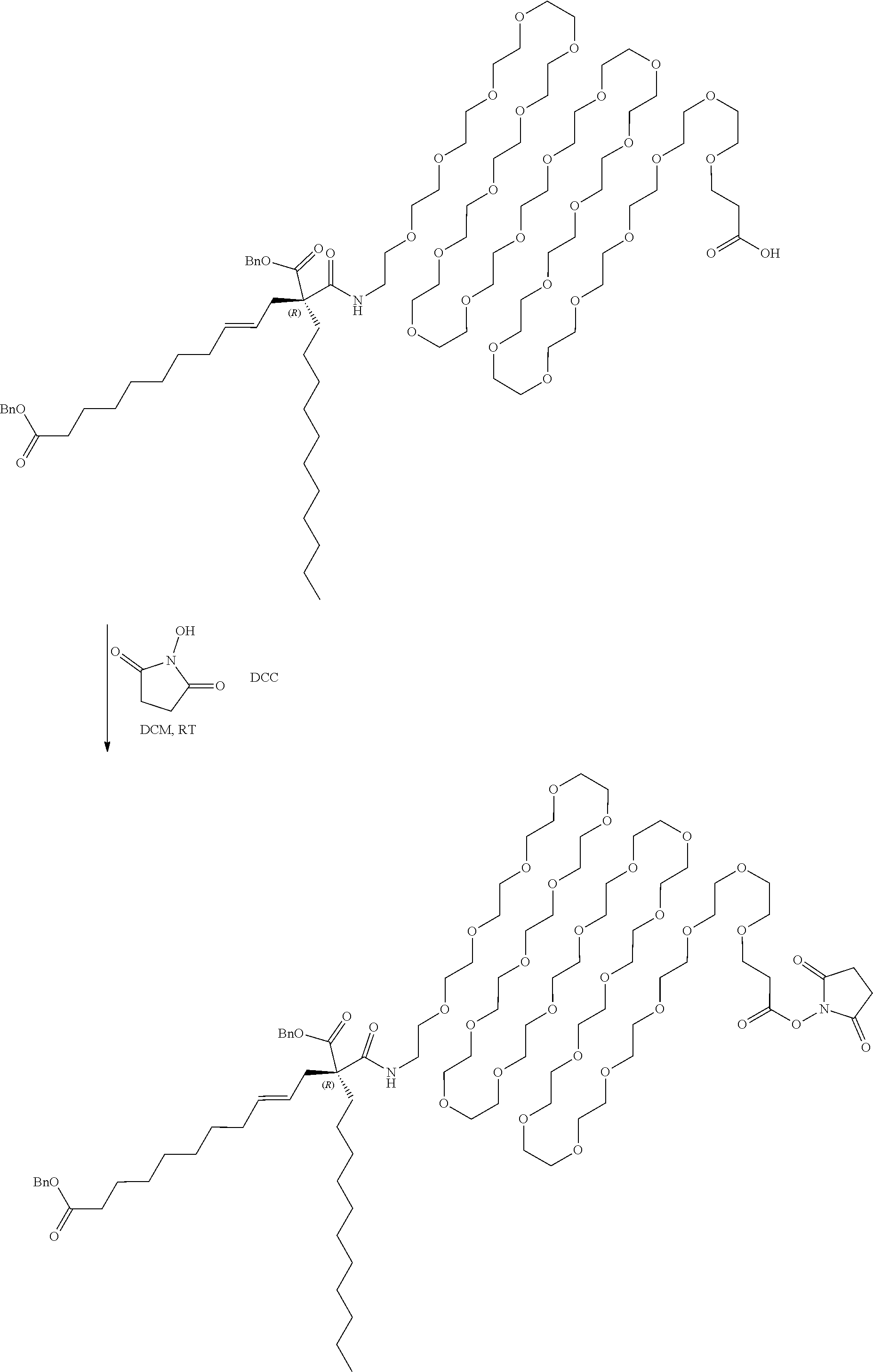

Intermediate 13B (312 mg, 0.178 mmol) was dissolved into 1.8 mL anhydrous DCM along with 1-hydroxypyrrolidine-2,5-dione (24.63 mg, 0.214 mmol) and then treated with 1 M DCC in DCM (Aldrich, 196 μL) which produced immediate precipitation of the dicyclohexyl urea byproduct. After 16 h, the reaction was complete and the reaction mixture was then injected directly onto a DCM equilibrated ISCO Gold 40 gram column and purified by NPLC (eluting 0-30% MeOH in DCM, silica). Fractions containing product were combined and concentrated to provide Intermediate 14B as a thick clear oil. LCMS Method F: Rt=4.21 min, [M+H+H₂O]⁺=1864.4.

### Intermediate 15B: (R)-2-((75-((2,5-dioxopyrrolidin-1-yl)oxy)-75-oxo-3,6,9,12,15,18,21,24,27,30,33,36,39,42,45,48,51,54,57,60,63,66,69,72-tetracosaoxapentaheptacontyl)carbamoyl)-2-undecyltridecanedioic acid

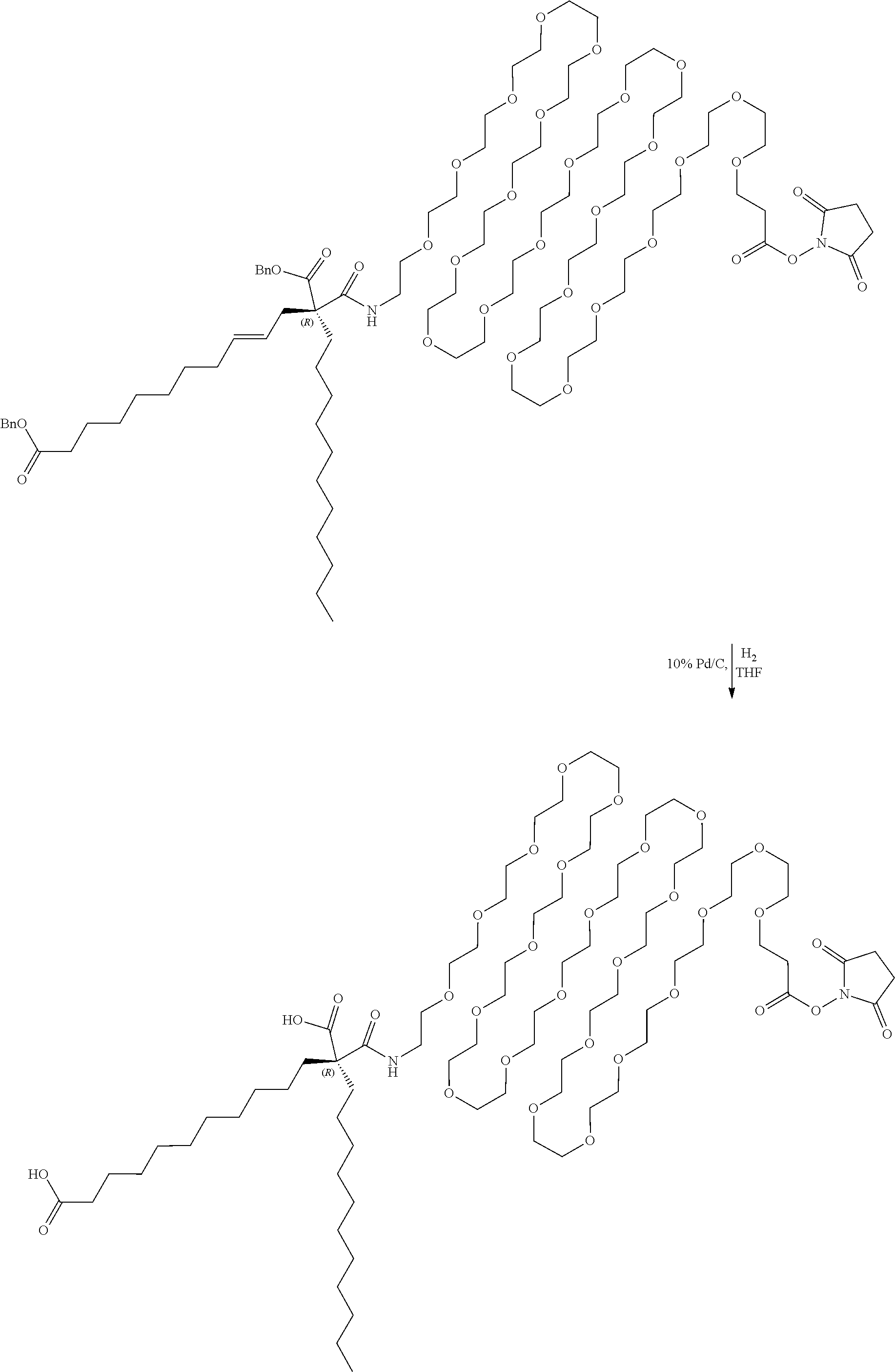

Intermediate 14B (172 mg, 0.093 mmol) was dissolved into 1.8 mL anhydrous THF and the atmosphere evacuated and replaced three times with nitrogen. To this mixture was added 10% palladium on carbon (10 mg, 9.4 umol) and the atmosphere evacuated and replaced with hydrogen from a balloon with magnetic stirring. After 16 h, the reaction was complete. The reaction mixture was filtered through Celite® after dilution with 5 mL anhydrous DCM. The palladium on carbon and the Celite-cake were washed twice with 5 mL DCM and filtered. All organics were combined and concentrated to provide Intermediate 15B. LCMS Method F: Rt=3.31 min. [M+H]⁺=1669.0.

### Intermediate 16: 1,11-Dibenzyl 11-(2,5-dioxocyclopentyl) docosane-1,11,11-tricarboxylate

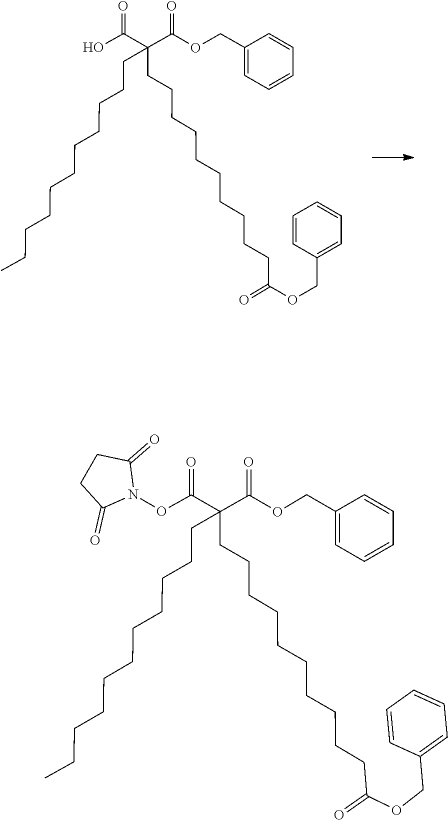 [SMILES: CCCCCCCCCCCC(CCCCCCCCCCC(=O)OCc1ccccc1)(C(=O)O)C(=O)OCc1ccccc1 ; CCCCCCCCCCCC(CCCCCCCCCCC(=O)OCc1ccccc1)(C(=O)OCc1ccccc1)C(=O)ON1C(=O)CCC1=O]

To a 1000 mL 3-neck round bottom flask (fitted with a mechanical stirrer and nitrogen inlet) was added Intermediate 3 (37.7 g, 60.5 mmol), DCM (360 mL, Ratio: 9.0), and THF (40 mL, Ratio: 1.0) followed by N-hydroxysuccinimide (7.31 g, 63.6 mmol) and DCC (14.99 g, 72.6 mmol). Five min after the addition, the resulting mixture had become a white suspension. The reaction mixture was stirred for a total of 6 h at RT and then filtered over a pad of Celite®. The pad was washed thoroughly with DCM (2 bed volumes). The combined organic phases were concentrated in vacuo, and the crude residue was dried under hi-vacuum. The crude product was isolated as a white oil. To the crude product was added DCM (˜400 mL) and silica gel (75 g). The resulting suspension was concentrated in vacuo and the residue was dried under hi-vacuum for 3 h. The batch was purified via column chromatography (750 g SiO₂ gel, eluting with 2% ethyl acetate/heptane to 35% ethyl acetate/heptane). The product containing fractions were combined, concentrated in vacuo and dried overnight under hi-vacuum to provide Intermediate 16 as a colorless oil. ¹H NMR (400 MHz, Chloroform-d) δ ppm 0.86-0.93 (m, 3H) 1.12-1.21 (m, 2H) 1.21-1.37 (m, 30H) 1.66 (quin, J=7.40 Hz, 2H) 1.89-2.07 (m, 4H) 2.37 (t, J=7.58 Hz, 2H) 2.84 (br. s., 4H) 5.13 (s, 2H) 5.25 (s, 2H) 7.30-7.47 (m, 10H).

### Intermediate 17: 14-((benzyloxy)carbonyl)-3,15-dioxo-1-phenyl-14-undecyl-2,19,22,25,28,31,34,37,40-nonaoxa-16-azatritetracontan-43-oic acid

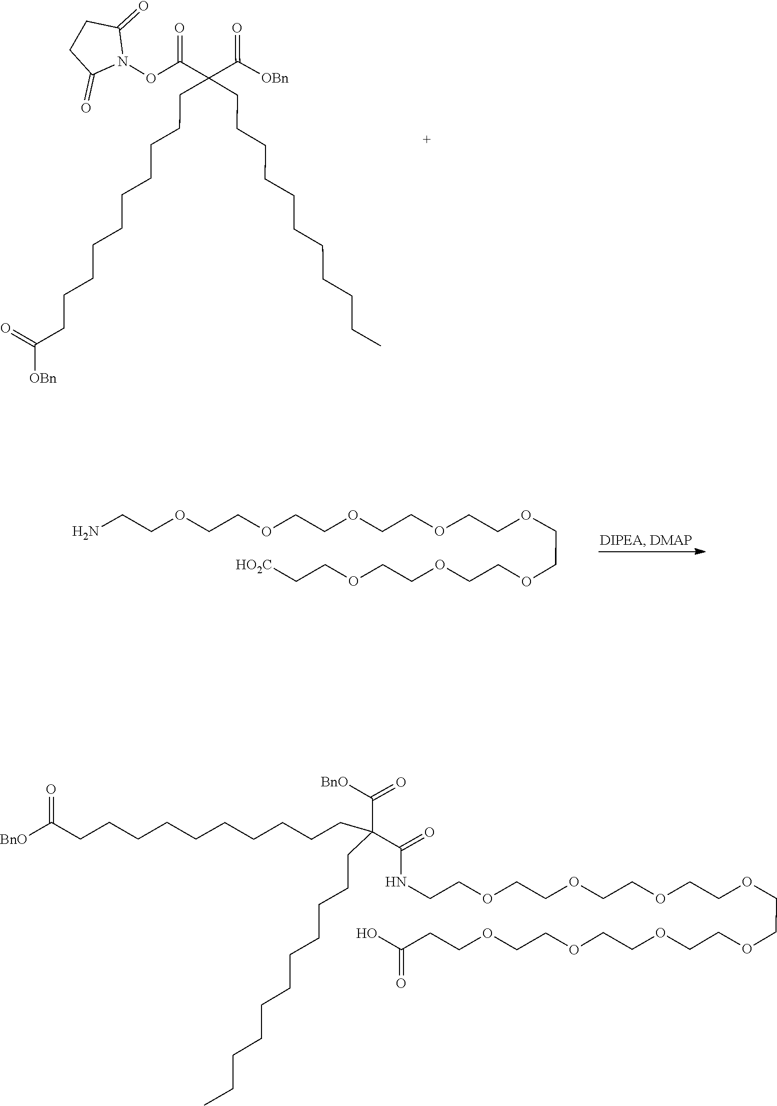 [SMILES: CCCCCCCCCCCC(CCCCCCCCCCC(=O)OCc1ccccc1)(C(=O)OCc1ccccc1)C(=O)ON1C(=O)CCC1=O ; NCCOCCOCCOCCOCCOCCOCCOCCOCCC(=O)O ; CCCCCCCCCCCC(CCCCCCCCCCC(=O)OCc1ccccc1)(C(=O)NCCOCCOCCOCCOCCOCCOCCOCCOCCC(=O)O)C(=O)OCc1ccccc1]

To a 250 mL round bottom flask (fitted with a magnetic stirrer and nitrogen inlet) was added Intermediate 16 (7.0 g, 9.72 mmol) and DCM (70 mL) followed by 1-amino-3,6,9,12,15,18,21,24-octaoxaheptacosan-27-oic acid (Amino-PEG8-Acid) (4.51 g, 10.21 mmol), DIPEA (4.25 mL, 24.31 mmol), and DMAP (0.119 g, 0.972 mmol). The resulting light yellow homogeneous solution was stirred overnight at ambient temperature. The reaction mixture was then concentrated in vacuo to provide a light yellow oily residue. This residue was then diluted with ethyl acetate (150 mL), and the solution was transferred to a 500 mL separatory funnel. The solution was then washed with brine (500 mL). The resulting aqueous phase was back-extracted with ethyl acetate (150 mL; then 100 mL). The combined organic phases were dried (with sodium sulfate), filtered over Celite®, and concentrated in vacuo. The crude product was purified via column chromatography (330 g SiO₂ gel, eluting with DCM to 10% methanol/DCM). The fractions containing the predominant product were combined and concentrated in vacuo. The residue was dried overnight under hi-vacuum to provide Intermediate 17. LCMS Method E: Rt=1.43 min. [M+H]⁺=1047.0

### Intermediate 18: 29,39-dibenzyl 1-(2,5-dioxopyrrolidin-1-yl) 28-oxo-29-undecyl-3,6,9,12,15,18,21,24-octaoxa-27-azanonatriacontane-1,29,39-tricarboxylate

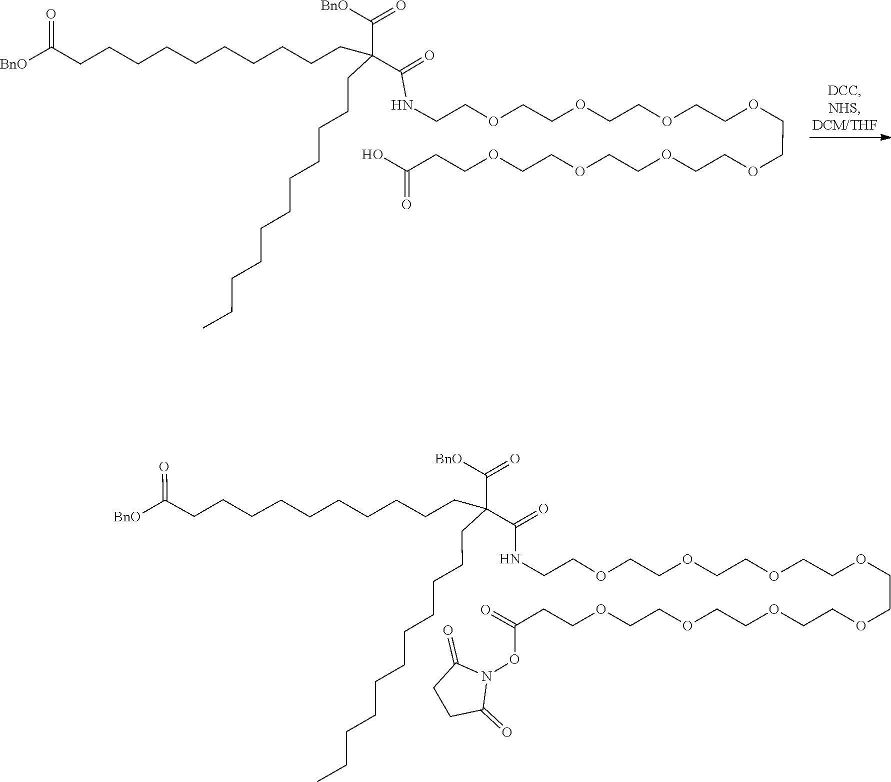 [SMILES: CCCCCCCCCCCC(CCCCCCCCCCC(=O)OCc1ccccc1)(C(=O)NCCOCCOCCOCCOCCOCCOCCOCCOCCC(=O)O)C(=O)OCc1ccccc1 ; CCCCCCCCCCCC(CCCCCCCCCCC(=O)OCc1ccccc1)(C(=O)NCCOCCOCCOCCOCCOCCOCCOCCOCCC(=O)ON1C(=O)CCC1=O)C(=O)OCc1ccccc1]

To a 50 mL round bottom flask containing Intermediate 17 (5.51 g, 5.27 mmol) was added DCM (27.5 mL, Ratio: 1.0) and THF (27.5 mL, Ratio: 1.0) followed by DCC (1.412 g, 6.85 mmol) and N-hydroxysuccinimide (0.697 g, 6.06 mmol). After stirring for approximately 10 min, the resulting mixture became a thick white suspension. The reaction mixture was then stirred for 3 h 45 min at ambient temperature and concentrated in vacuo to provide a white paste. To the mixture was added DCM (35 mL), and the resulting white suspension was stirred for 10 min. The mixture was then filtered over a pad of Celite® and the pad was washed with cold DCM (one bed volume). The combined filtrates were concentrated in vacuo. The residue was dried overnight under hi-vacuum to provide Intermediate 18 as a colorless oil. LCMS Method E: Rt=1.45 min. [M+H]⁺=1044.0.

### Intermediate 19: 2-((27-((2,5-dioxopyrrolidin-1-yl)oxy)-27-oxo-3,6,9,12,15,18,21,24-octaoxaheptacosyl)carbamoyl)-2-undecyltridecanedioic acid

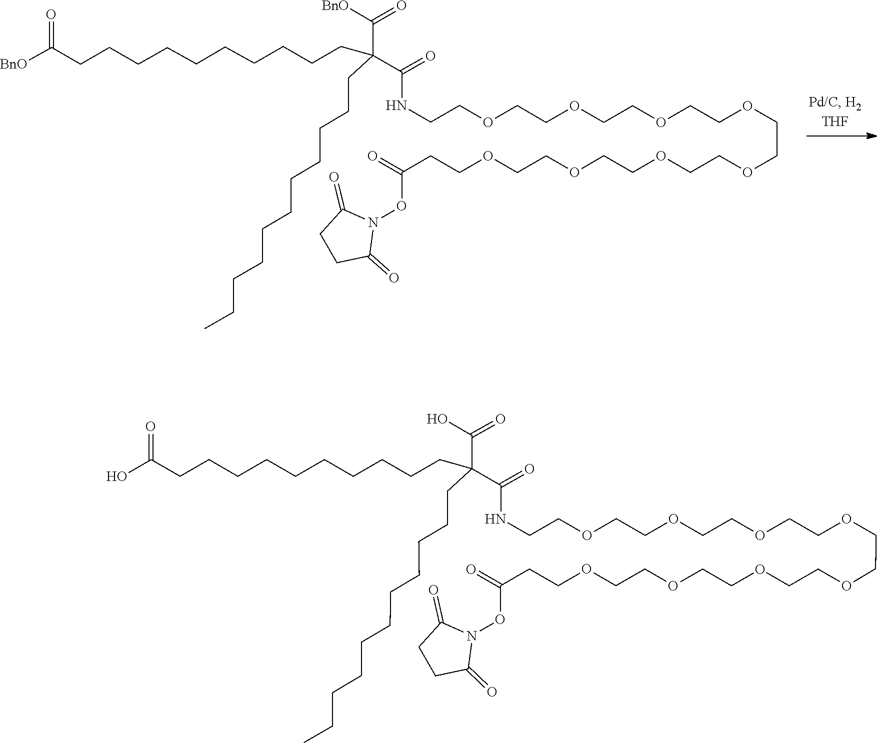 [SMILES: CCCCCCCCCCCC(CCCCCCCCCCC(=O)OCc1ccccc1)(C(=O)NCCOCCOCCOCCOCCOCCOCCOCCOCCC(=O)ON1C(=O)CCC1=O)C(=O)OCc1ccccc1 ; CCCCCCCCCCCC(CCCCCCCCCCC(=O)O)(C(=O)O)C(=O)NCCOCCOCCOCCOCCOCCOCCOCCOCCC(=O)ON1C(=O)CCC1=O]

To a 250 mL round bottom flask (fitted with a magnetic stirrer) was added Intermediate 18 (6.0 g, 5.25 mmol) and THF (70 ml). To this solution was added 10% Pd/C (0.603 g, 0.567 mmol), and the reaction vessel was purged with nitrogen followed by hydrogen. The resulting mixture was then exposed to hydrogen (balloon pressure) for 3 h. The reaction vessel was purged with nitrogen and the suspension was filtered over a pad of Celite®. The pad was washed thoroughly with THF and the combined filtrates were concentrated in vacuo. The resulting residue was then dried overnight under hi-vacuum to provide Intermediate 19 as a colorless oil. LCMS Method E: Rt=0.91 min. [M+H]⁺=963.8.

### Intermediate 20: 14-((benzyloxy)carbonyl)-3,15-dioxo-1-phenyl-14-undecyl-2,19,22-trioxa-16-azapentacosan-25-oic acid

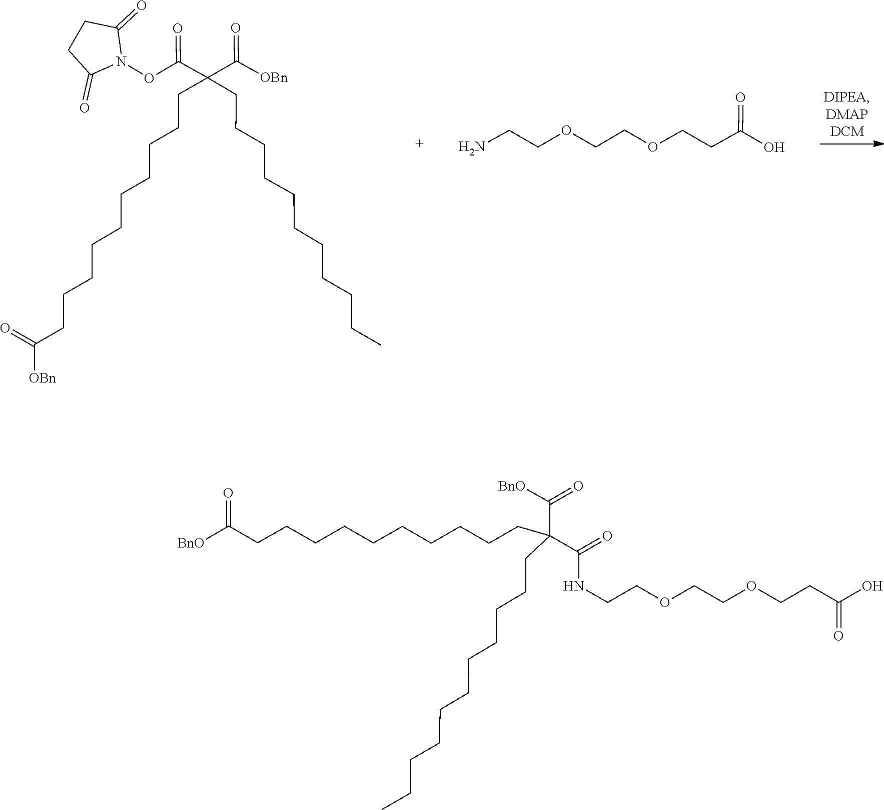 [SMILES: CCCCCCCCCCCC(CCCCCCCCCCC(=O)OCc1ccccc1)(C(=O)OCc1ccccc1)C(=O)ON1C(=O)CCC1=O ; NCCOCCOCCC(=O)O ; CCCCCCCCCCCC(CCCCCCCCCCC(=O)OCc1ccccc1)(C(=O)NCCOCCOCCC(=O)O)C(=O)OCc1ccccc1]

To a 250 mL round bottom flask (with a magnetic stir bar) was added Intermediate 16 (5.0 g, 6.94 mmol) and DCM (Volume: 100 mL) followed by 3-(2-(2-aminoethoxy)ethoxy)propanoic acid (Amino-PEG2-Acid, 1.231 g, 6.94 mmol), DIPEA (3.03 mL, 17.36 mmol), and DMAP (0.085 g, 0.694 mmol). The resulting white suspension was stirred for 22 h at ambient temperature. LCMS indicated significant NHS ester starting material remaining. The reaction mixture was then warmed to 40° C. and stirring was continued for 5.5 h. The mixture was cooled to ambient temperature and then concentrated in vacuo to provide a white paste. To the mixture was added ethyl acetate (150 mL). The solution was transferred to a 500 mL separatory funnel along with brine (150 mL). The phases were separated and the aqueous phase was extracted with ethyl acetate (150 mL) twice. The combined organic phases were dried (with sodium sulfate), filtered over Celite®, and concentrated in vacuo. The crude product was purified via column chromatography (120 g silica gel, eluting with 0.5% methanol/DCM to 60% methanol/DCM). The fractions containing the predominant product by TLC (5% methanol/DCM) were combined, concentrated in vacuo, and the resulting residue was dried under hi-vacuum overnight to provide Intermediate 20 as a colorless oil. LCMS Method E: Rt=1.45 min. [M+H]⁺=782.8.

### Intermediate 21: dibenzyl 2-((2-(2-(3-((2,5-dioxopyrrolidin-1-yl)oxy)-3-oxopropoxy)ethoxy)ethyl)carbamoyl)-2-undecyltridecanedioate

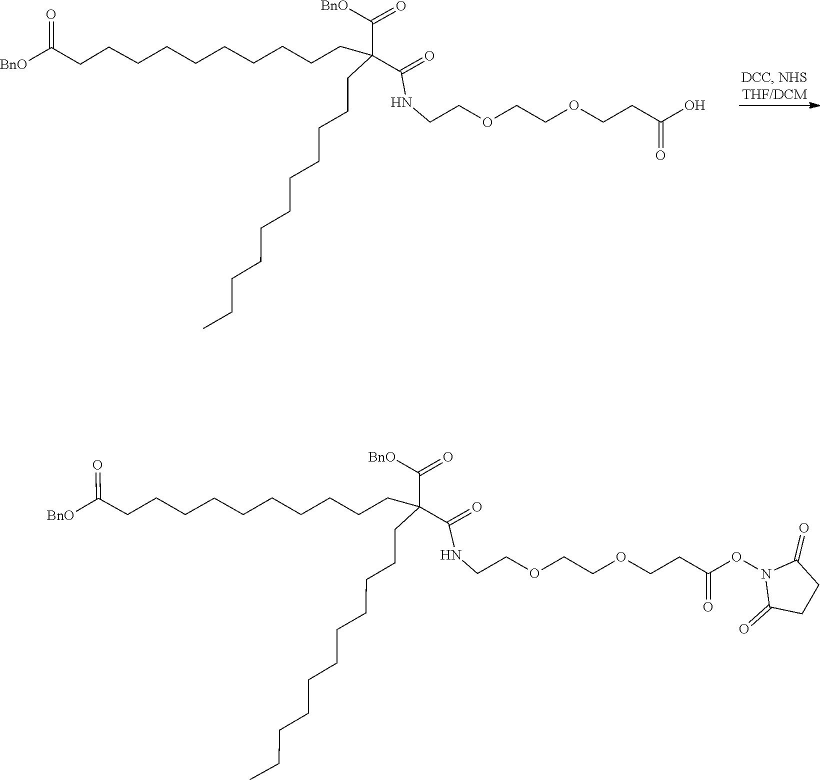 [SMILES: CCCCCCCCCCCC(CCCCCCCCCCC(=O)OCc1ccccc1)(C(=O)NCCOCCOCCC(=O)O)C(=O)OCc1ccccc1 ; CCCCCCCCCCCC(CCCCCCCCCCC(=O)OCc1ccccc1)(C(=O)NCCOCCOCCC(=O)ON1C(=O)CCC1=O)C(=O)OCc1ccccc1]

To a 500 mL round bottom flask (fitted with a magnetic stirrer and nitrogen inlet) was added Intermediate 20 (3.7 g, 4.73 mmol), DCM (19 mL, Ratio: 1.0) and THF (19 mL, Ratio: 1.0) followed by N-hydroxysuccinimide (0.626 g, 5.44 mmol) and DCC (1.269 g, 6.15 mmol). The resulting mixture was stirred for 3 h at ambient temperature, at which time it had become a white suspension. The suspension was then filtered over Celite®, and the pad was washed with DCM. The combined filtrates were concentrated in vacuo. The resulting residue was then suspended in DCM (20 mL) and the mixture was stirred for 10 min at ambient temperature, and then filtered over a pad of Celite®. The pad was washed with cold DCM. The combined filtrates were concentrated in vacuo. The residue was dried overnight under hi-vacuum to provide Intermediate 21 as light yellow oil. LCMS Method E: Rt=1.47 min, [M+H]⁺=879.7.

### Intermediate 22: 2-((2-(2-(3-((2,5-dioxopyrrolidin-1-yl)oxy)-3-oxopropoxy)ethoxy)ethyl)carbamoyl)-2-undecyltridecanedioic acid

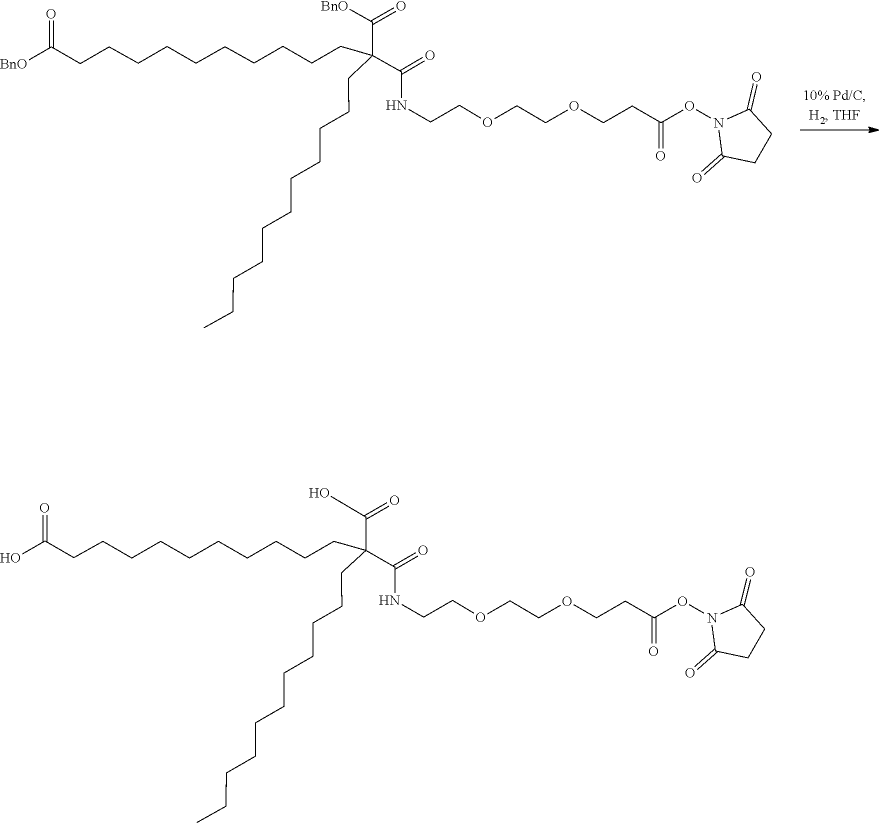 [SMILES: CCCCCCCCCCCC(CCCCCCCCCCC(=O)OCc1ccccc1)(C(=O)NCCOCCOCCC(=O)ON1C(=O)CCC1=O)C(=O)OCc1ccccc1 ; CCCCCCCCCCCC(CCCCCCCCCCC(=O)O)(C(=O)O)C(=O)NCCOCCOCCC(=O)ON1C(=O)CCC1=O]

To a 100 mL round bottom flask containing Intermediate 21 (4.16 g, 4.73 mmol) was added THF (40 mL). To this solution was added 10% Pd/C (0.42 g, 3.93 mmol), and the vessel was purged with nitrogen. The reaction vessel was then purged with hydrogen and exposed to hydrogen pressure (balloon). The resulting black suspension was stirred for 4 h and then filtered over a pad of Celite®. The pad was washed with THF. The combined filtrates were concentrated in vacuo and then dried under hi-vacuum to provide Intermediate 22 as colorless oil. LCMS Method G: Rt=1.72 min, [M+H]⁺=699.4.

### Intermediate 23: Benzyl 11-bromoundecanoate

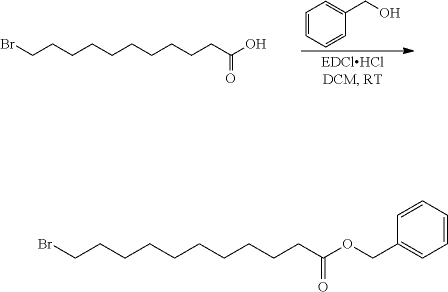 [SMILES: O=C(O)CCCCCCCCCCBr ; OCc1ccccc1 ; O=C(CCCCCCCCCCBr)OCc1ccccc1]

To a 2 L round 3-neck round bottom flask (fitted with a mechanical stirrer, temperature probe, and nitrogen inlet) was added 11-bromoundecanoic acid (50 g, 189 mmol) and 500 mL dichloromethane. To the resulting orange homogeneous solution was then added benzyl alcohol (23.53 mL, 226 mmol), EDCI-HCl (54.2 g, 283 mmol), and DMAP (1.152 g, 9.43 mmol). The reaction mixture was stirred overnight. TLC analysis (30% ethyl acetate in heptane) indicated consumption of the 11-bromoundecanoic acid. The reaction mixture was transferred to a 2 L round bottom flask and concentrated in vacuo. The resulting residue was diluted with 1 L water and 800 mL MTBE. The phases were separated, and the aqueous phase was extracted twice with 600 mL MTBE. The combined organic phases were washed with 750 mL brine, dried with sodium sulfate, filtered over Celite®, and concentrated in vacuo. The material was dried under hi-vacuum for 2 h to provide a light yellow oil. The crude product was dissolved in 500 mL DCM and 100 g silica gel was added. The mixture was concentrated in vacuo and then dried overnight under hi-vacuum. The residue was purified via chromatography (750 g silica column, eluting with 1% EtOAc/heptane to 20% EtOAc/heptane gradient). The benzyl 11-bromoundecanoate containing fractions were combined and concentrated in vacuo. The residue was dried under hi-vacuum for 5 h to provide Intermediate 23 as colorless oil. ¹H NMR (400 MHz, Chloroform-d) δ 7.58-7.31 (m, 5H), 5.14 (s, 2H), 3.43 (t, J=6.9 Hz, 2H), 2.38 (t, J=7.5 Hz, 2H), 1.87 (p, J=7.0 Hz, 2H), 1.73-1.61 (m, 2H), 1.49-1.40 (m, 2H), 1.37-1.26 (m, 10H).

### Intermediate 24: 1,11,21-Tribenzyl 11-tert-butyl henicosane-1,11,11,21-tetracarboxylate

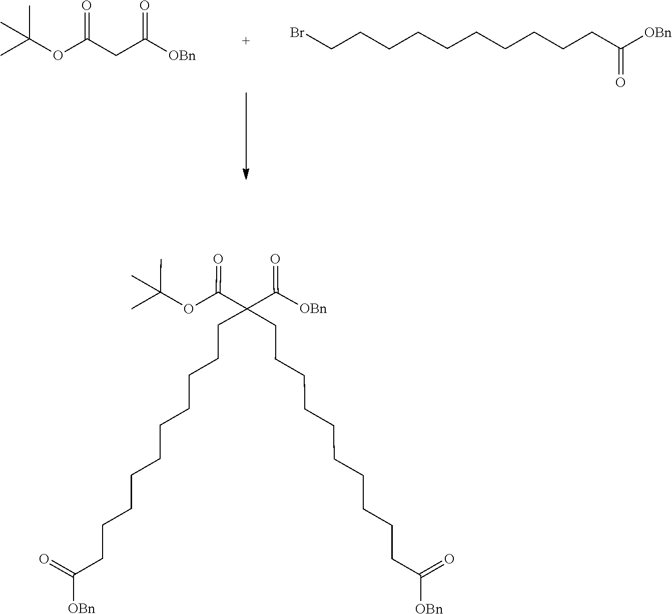 [SMILES: CC(C)(C)OC(=O)CC(=O)OCc1ccccc1 ; O=C(CCCCCCCCCCBr)OCc1ccccc1 ; CC(C)(C)OC(=O)C(CCCCCCCCCCC(=O)OCc1ccccc1)(CCCCCCCCCCC(=O)OCc1ccccc1)C(=O)OCc1ccccc1]

To a 250 mL 3-neck round bottom flask fitted with a mechanical stirrer, temperature probe, and nitrogen inlet was added benzyl tert-butyl malonate (6 g, 23.97 mmol) and 30 mL DMF followed by a mixture of Intermediate 23 (18.74 g, 52.7 mmol) and 60 mL DMF. To this colorless solution was added cesium carbonate (31.2 g, 96 mmol) and the resulting suspension was stirred at ambient temperature. After stirring for 5.5 h at ambient temperature, LCMS indicated no benzyl tert-butyl malonate was present. The reaction mixture was a mixture of the mono- and di-alkylated products. The mixture was therefore allowed to stir for 22 h, but LCMS indicated monoalkylation intermediate was still remaining. The reaction mixture was then heated to 40° C. and stirred for 3 h. LCMS still indicated minimal progress. The mixture was cooled to 0-5° C. and 200 mL deionized (DI) water was added in a thin stream. The mixture was then warmed to ambient temperature and transferred to a 500 mL separatory funnel. The aqueous phase was extracted twice with 200 mL MTBE. The combined organic phases were washed with 200 mL brine, dried sodium sulfate, filtered over Celite®, and concentrated in vacuo. The residue was dried under hi-vacuum for 2 h to provide a crude colorless product that was purified via NPLC (330 g ISCO silica column, eluting 0.5% ethyl acetate/heptane to 30% ethyl acetate/heptane gradient). The product containing fractions were combined and concentrated in vacuo. The residue was dried overnight under hi-vacuum to provide Intermediate 24 as colorless oil. LCMS Method E: Rt=1.75 min, [M+H+H₂O]⁺=821.3.

### Intermediate 25: 13-(Benzyloxy)-2-(11-(benzyloxy)-11-oxoundecyl)-2-((benzyloxy)carbonyl)-13-oxotridecanoic acid

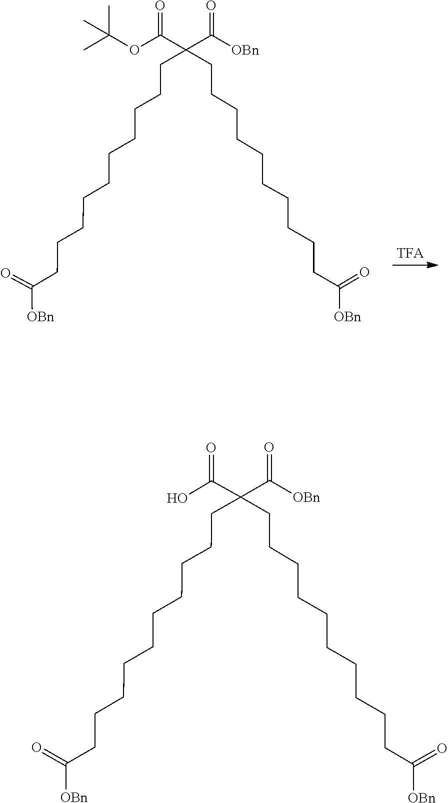 [SMILES: CC(C)(C)OC(=O)C(CCCCCCCCCCC(=O)OCc1ccccc1)(CCCCCCCCCCC(=O)OCc1ccccc1)C(=O)OCc1ccccc1 ; O=C(CCCCCCCCCCC(CCCCCCCCCCC(=O)OCc1ccccc1)(C(=O)O)C(=O)OCc1ccccc1)OCc1ccccc1]

To a 1 L round bottom flask fitted with a magnetic stir bar and nitrogen inlet was added Intermediate 24 (17.78 g, 22.25 mmol) and 180 mL TFA and the resulting mixture was stirred for 45 min. Once LCMS analysis indicated no starting material remaining, the mixture was concentrated in vacuo to provide a light yellow oil. The resulting oil was diluted with 250 mL toluene and was then concentrated in vacuo to remove any remaining TFA. This last step was repeated once. The residue was dried under hi-vacuum over the weekend to provide Intermediate 25 as a light yellow oil, which was used as is in the next step. LCMS Method E: Rt=1.55 min. [M+H+H₂O]⁺=760.4.

### Intermediate 26: 1,11,21-tribenzyl 11-(2,5-dioxopyrrolidin-1-yl) henicosane-1,11,11,21-tetracarboxylate

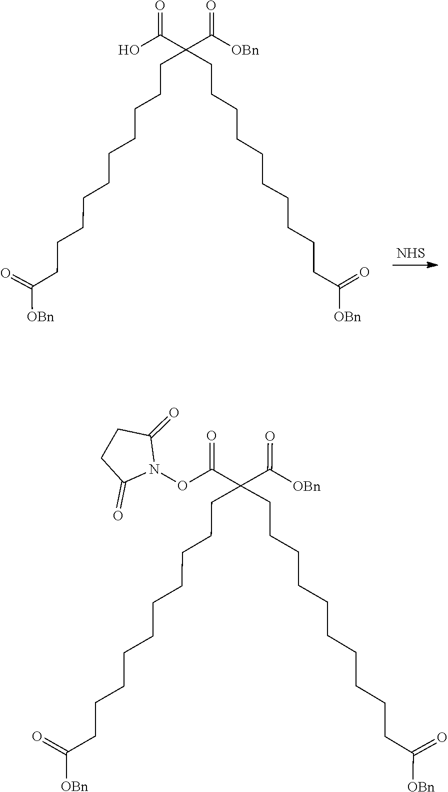

To a 500 mL round bottom flask containing Intermediate 25 (16.53 g, 22.25 mmol) was added 180 mL DCM and 20 mL THF, followed by N-hydroxysuccinimide (2.69 g, 23.36 mmol) and DCC (5.51 g, 26.7 mmol). The resulting mixture was stirred overnight after which the LCMS indicated complete conversion to the desired product. The resulting white suspension was filtered over a pad of Celite® and the pad was washed with two bed volumes DCM. The combined filtrates were concentrated in vacuo to provide a colorless oil, and the resulting oil was dried under hi-vacuum for 1 h to provide 21.3 g of the crude product. The crude product was dissolved in 250 mL DCM and 32 g silica gel was added. The mixture was concentrated in vacuo and then dried under hi-vacuum for 2 h. The residue was purified via cartridge dry loaded NPLC (330 g silica gel column, eluting with 5% ethyl acetate/heptane to 40% ethyl acetate/heptane gradient). The product containing fractions were combined, concentrated in vacuo, and dried overnight under hi-vacuum to provide Intermediate 26 as colorless oil. LCMS Method E: Rt=1.58 min. [M+H+H₂O]⁺=857.4.

### Intermediate 27: 14-(11-(benzyloxy)-11-oxoundecyl)-14-((benzyloxy)carbonyl)-3,15-dioxo-1-phenyl-2,19,22,25,28,31,34,37,40,43,46,49,52,55,58,61,64,67,70,73,76,79,82,85,88-pentacosaoxa-16-azahennonacontan-91-oic acid

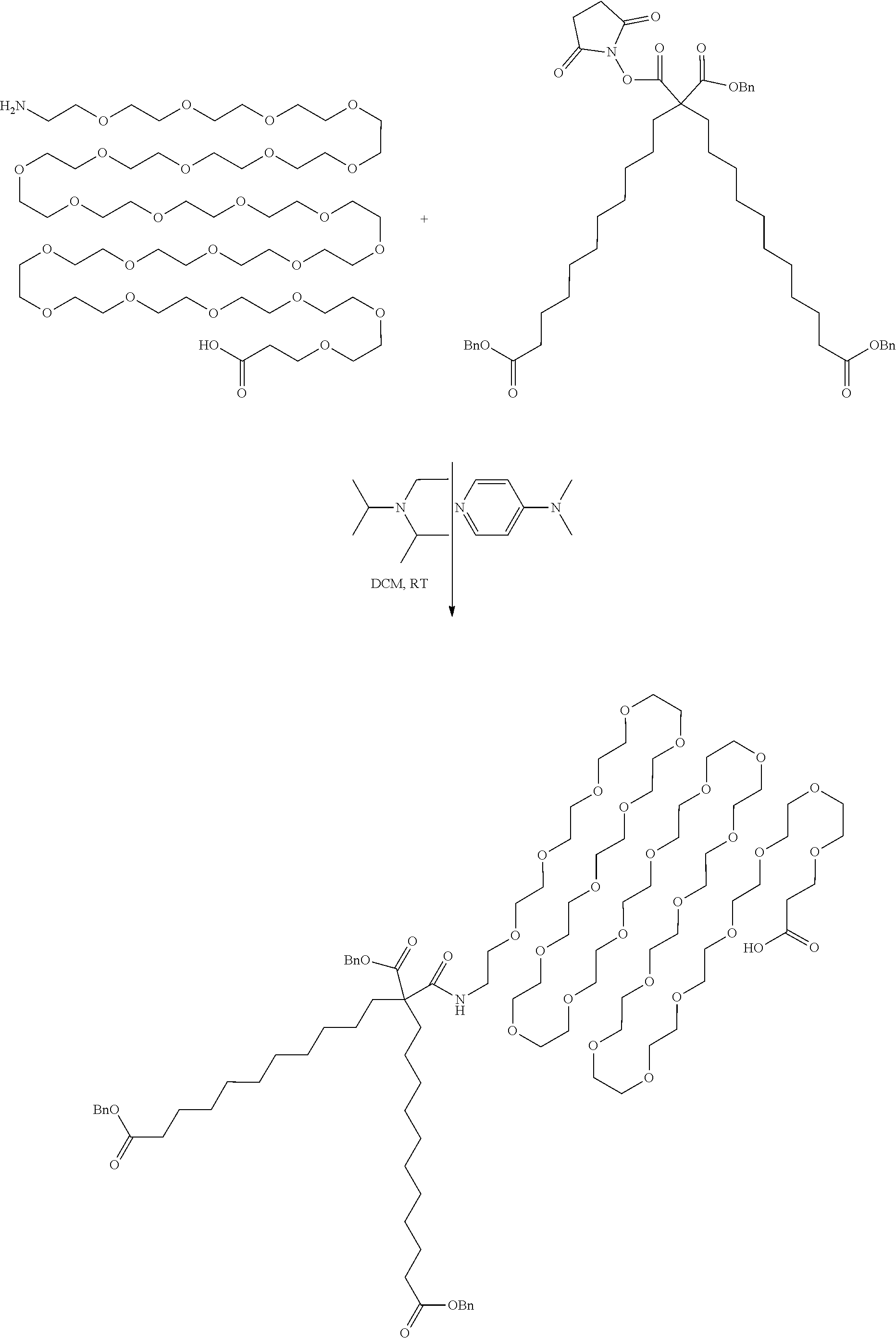

Intermediate 26 (479 mg, 0.503 mmol) was dissolved in 5.7 mL anhydrous DCM. This solution was then treated with 1-amino 3,6,9,12,15,18,21,24,27,30,33,36,39,42,45,48,51,54,57,60,63,66,69,72-tetracosaoxapentaheptacontan-75-oic acid (686 mg, 0.599 mmol), DIPEA (149 μL, 0.855 mmol) and DMAP (7 mg, 0.057 mmol) and stirred at room temperature for 16 h. After 16 h, the reaction was complete by LC/MS analysis and the volatiles were removed by rotovap. The crude product was purified by NPLC (24 gram ISCO Gold silica column, eluting with 0-20% MeOH in DCM) to yield Intermediate 27 as a thick oil. LCMS Method E: Rt=1.35 min. [M+H]⁺=1871.9.

### Intermediate 28: 77,87-dibenzyl 1-(2,5-dioxopyrrolidin-1-yl) 77-(11-(benzyloxy)-11-oxoundecyl)-76-oxo-3,6,9,12,15,18,21,24,27,30,33,36,39,42,45,48,51,54,57,60,63,66,69,72-tetracosaoxa-75-azaheptaoctacontane-1,77,87-tricarboxylate

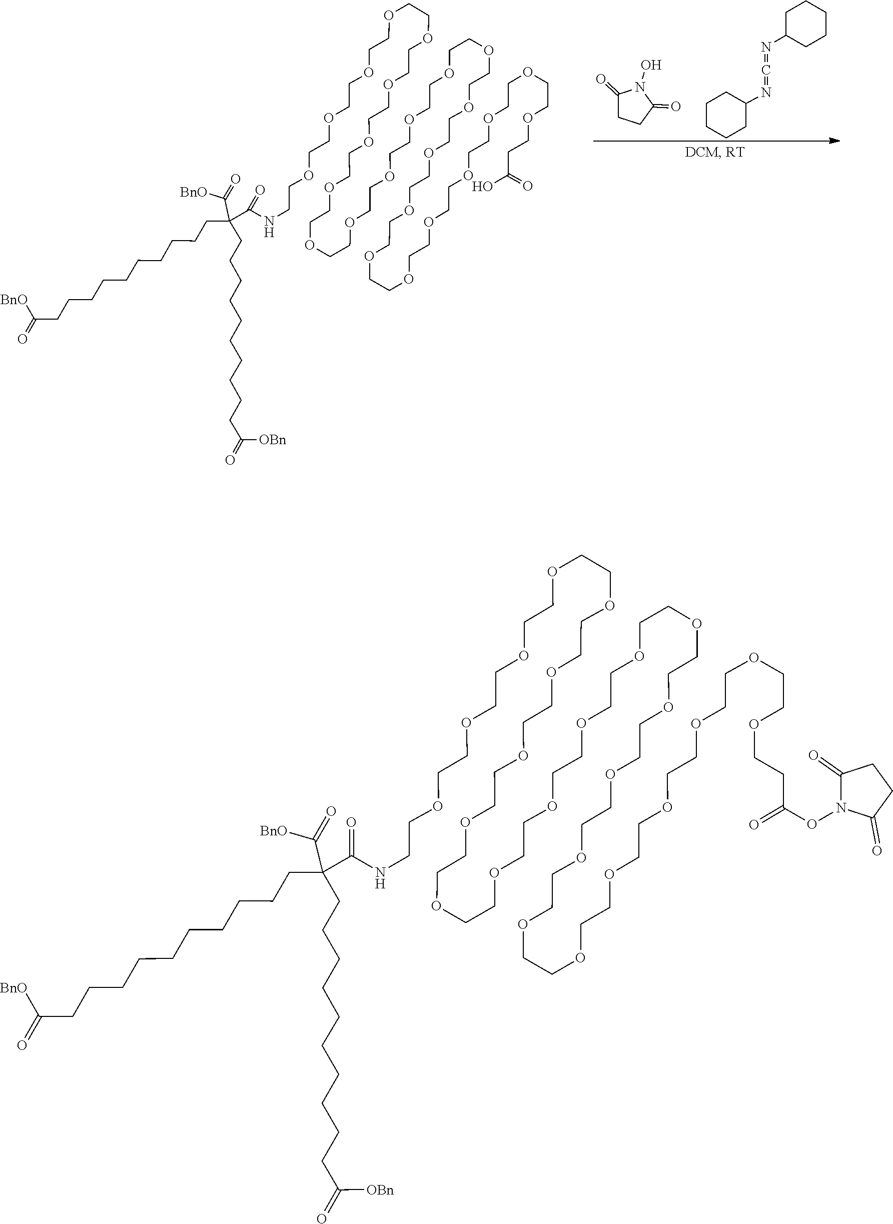

Intermediate 27 (764 mg, 0.408 mmol) was treated with 1-hydroxypyrrolidine-2,5-dione (56.4 mg, 0.490 mmol) in 4 mL DCM. To this mixture was added a solution of 1M DCC in DCM (Aldrich, 0.499 mL, 0.499 mmol) and the reaction mixture was allowed to stir under nitrogen. After 16 h, the reaction was complete. Volatiles were removed and the residue purified by NPLC (24 gram ISCO Gold column, eluting with 0-15% MeOH in DCM). Fractions containing product were combined and concentrated to yield Intermediate 28. LCMS Method F: Rt=4.03 min. [M+2H]²⁺=985.1.

### Intermediate 29: 11-((75-((2,5-dioxopyrrolidin-1-yl)oxy)-75-oxo-3,6,9,12,15,18,21,24,27,30,33,36,39,42,45,48,51,54,57,60,63,66,69,72-tetracosaoxapentaheptacontyl)carbamoyl)henicosane-1,11,21-tricarboxylic acid

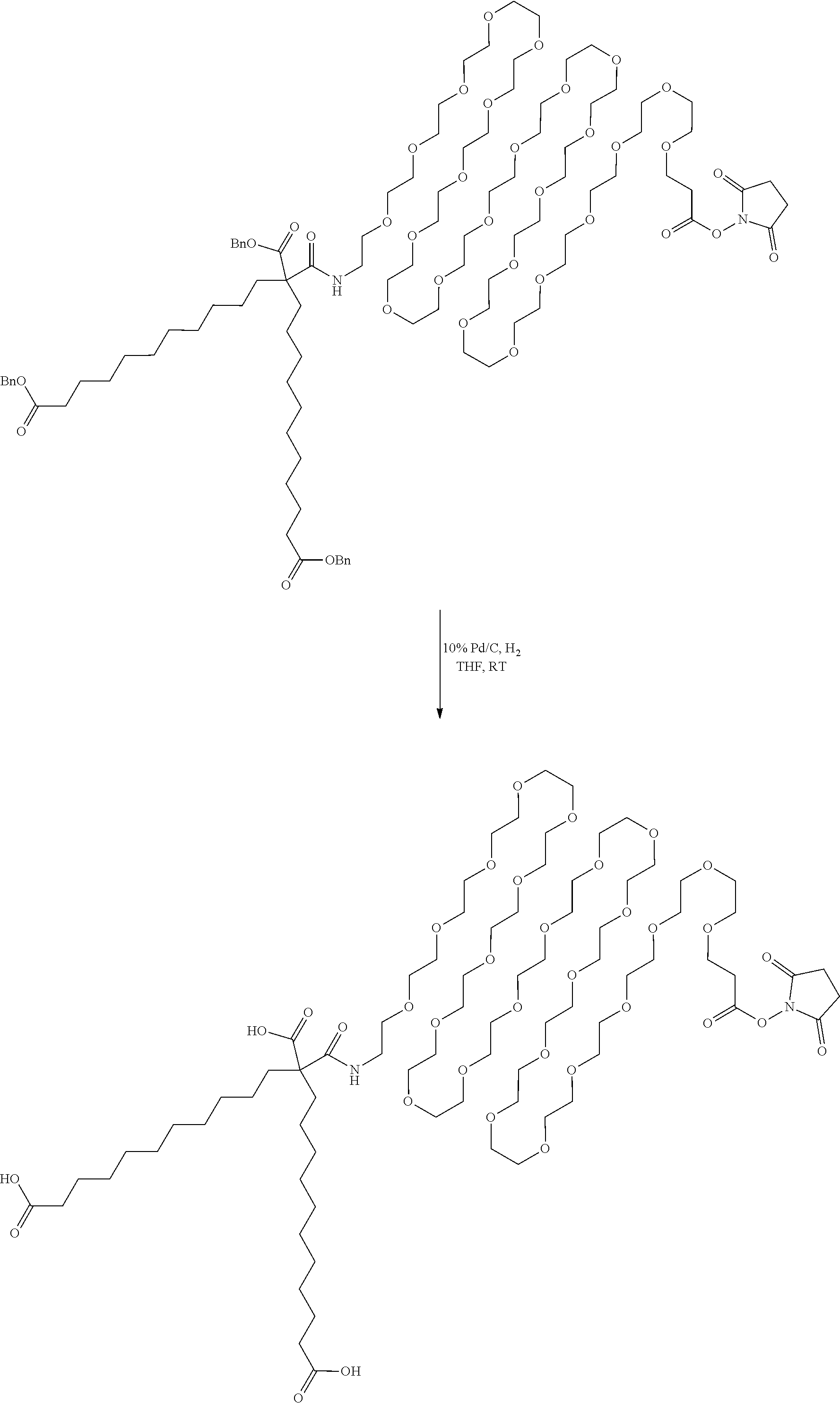 [SMILES: O=C(CCCCCCCCCCC(CCCCCCCCCCC(=O)OCc1ccccc1)(C(=O)NCCOCCOCCOCCOCCOCCOCCOCCOCCOCCOCCOCCOCCOCCOCCOCCOCCOCCOCCOCCOCCOCCOCCOCCOCCC(=O)ON1C(=O)CCC1=O)C(=O)OCc1ccccc1)OCc1ccccc1 ; O=C(O)CCCCCCCCCCC(CCCCCCCCCCC(=O)O)(C(=O)O)C(=O)NCCOCCOCCOCCOCCOCCOCCOCCOCCOCCOCCOCCOCCOCCOCCOCCOCCOCCOCCOCCOCCOCCOCCOCCOCCC(=O)ON1C(=O)CCC1=O]

Intermediate 28 (500 mg, 0.254 mmol) was dissolved into 2.5 mL anhydrous THF with a stirring bar. The atmosphere was evacuated and replaced with nitrogen three times. 10% palladium on carbon (Aldrich, 27 mg, 0.025 mmol) was then added carefully and the flask evacuated. The atmosphere was replaced with hydrogen from a balloon reservoir. The reaction mixture was allowed to stir overnight for 16 h where upon LC/MS indicated the reaction was complete. The reaction mixture was diluted with 5 mL anhydrous DCM and filtered through Celite®. The filtrate was concentrated to provide Intermediate 29 as a thick clear oil. LCMS Method F: Rt=2.60 min, [M+2H]²⁺=849.9.

### Intermediate 30: Benzyl 12-bromododecanoate

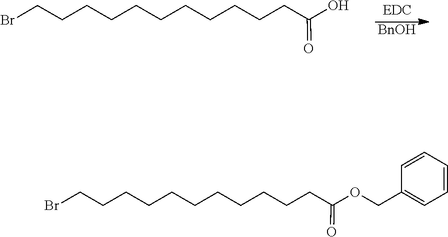 [SMILES: O=C(O)CCCCCCCCCCCBr ; "BnOH" ; O=C(CCCCCCCCCCCBr)OCc1ccccc1]

12-bromododecanoic acid (2 g, 7.16 mmol), benzyl alcohol (1.16 g, 10.74 mmol) and EDC-HCl (2.06 g, 10.74 mmol) were combined in 24 mL DCM. To this solution was added DMAP (44 mg, 0.358 mmol) in a single portion and the resulting mixture was allowed to stir overnight. The reaction was ˜90% complete based on LCMS analysis. The reaction mixture was purified by NPLC (eluting with 0-15% EtOAc in heptane, silica). Fractions containing product were combined and concentrated to provide desired product, Intermediate 30. ¹H NMR: (400 MHz, Chloroform-d) δ 7.47-7.31 (m, 5H), 5.14 (s, 2H), 3.43 (t, J=6.9 Hz, 2H), 2.38 (t, J=7.5 Hz, 2H), 1.88 (p, 2H), 1.67 (p, 2H), 1.50-1.39 (m, 2H), 1.35-1.26 (m, 12H).

### Intermediate 31: 1,12,23-tribenzyl 12-(tert-butyl) tricosane-1,12,12,23-tetracarboxylate, 1,12-dibenzyl 1-(tert-butyl) dodecane-1,1,12-tricarboxylate

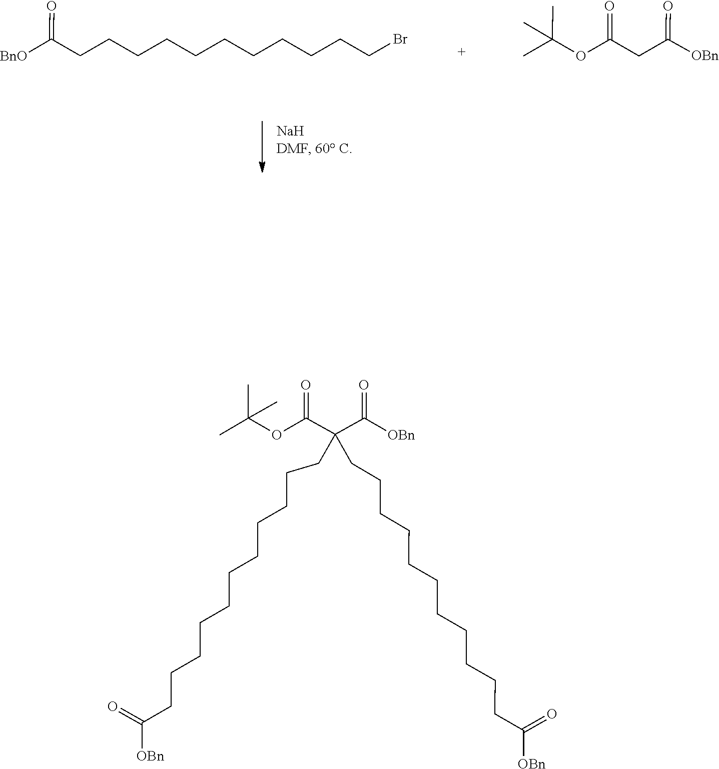 [SMILES: O=C(CCCCCCCCCCCBr)OCc1ccccc1 ; CC(C)(C)OC(=O)CC(=O)OCc1ccccc1 ; CC(C)(C)OC(=O)C(CCCCCCCCCCCC(=O)OCc1ccccc1)(CCCCCCCCCCCC(=O)OCc1ccccc1)C(=O)OCc1ccccc1]

Intermediate 30 (1 g, 2.71 mmol), benzyl tert-butyl malonate (276.4 mg, 1.104 mmol) and sodium hydride 60% in oil (97 mg, 2.43 mmol) were combined in 12 mL anhydrous DMF and the resulting mixture was stirred at RT overnight under a nitrogen atmosphere in an oven dried round bottom flask. After 16 h, LCMS analysis indicated complete conversion to the desired product. The reaction mixture was partitioned between water and EtOAc and washed with EtOAc (2×20 mL). The organic phases were combined, washed with brine, dried with sodium sulfate, filtered and concentrated by rotovap. The crude product was purified by NPLC (eluting with 0-60% EtOAc in heptane, silica, ELSD detection). Excess SM bromide eluted first and quickly, mono alkylated product eluted second followed by desired product. Fractions containing product were combined and concentrated to provide Intermediate 31 as a clear viscous oil. LCMS Method E: Rt=1.73 min, [M+H+H₂O]⁺=845.0.

### Intermediate 32: 14-(benzyloxy)-2-(12-(benzyloxy)-12-oxododecyl)-2-((benzyloxy)carbonyl)-14-oxotetradecanoic acid

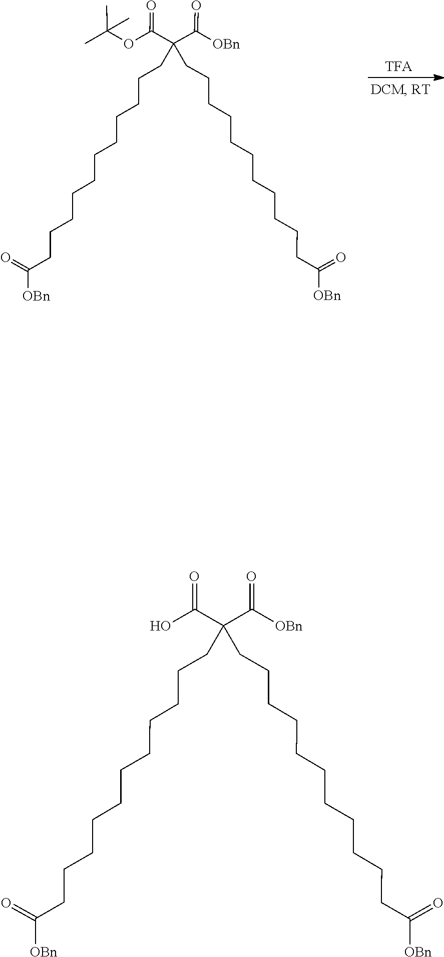 [SMILES: CC(C)(C)OC(=O)C(CCCCCCCCCCCC(=O)OCc1ccccc1)(CCCCCCCCCCCC(=O)OCc1ccccc1)C(=O)OCc1ccccc1 ; O=C(CCCCCCCCCCCC(CCCCCCCCCCCC(=O)OCc1ccccc1)(C(=O)O)C(=O)OCc1ccccc1)OCc1ccccc1]

Intermediate 31 (650 mg, 0.786 mmol) was dissolved in DCM (7.2 mL) and treated with TFA (0.605 mL, 7.86 mmol). After 16 h, the reaction was complete as indicated by LC/MS ELSD signal. Volatiles were removed and the resulting residue was purified by NPLC (eluting with 0-5% MeOH in DCM, silica). Fractions containing product were combined and concentrated to provide Intermediate 32 as a clear oil. LCMS Method E: Rt=1.57 min, [M+H]⁺+771.9.

### Intermediate 33: 1,12,23-tribenzyl 12-(2,5-dioxopyrrolidin-1-yl) tricosane-1,12,12,23-tetracarboxylate

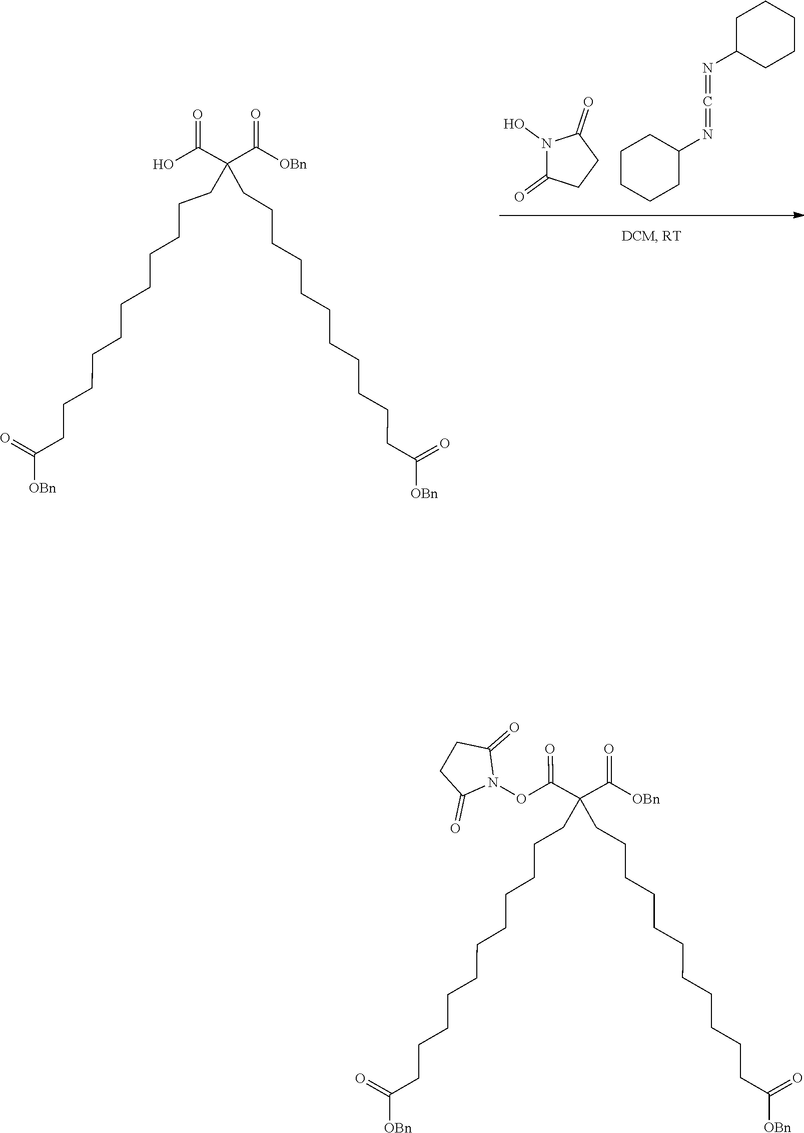

Intermediate 32 (310 mg, 0.402 mmol) was dissolved into 3.6 mL DCM along with 1-hydroxypyrrolidine-2,5-dione (50.9 mg, 0.442 mmol) and 1 M DCC in DCM (Aldrich, 422 μl, 0.422 mmol). After 15 min, the precipitation of DCU byproduct was observed. The reaction mixture was allowed to stir overnight after which LC/MS indicated complete conversion to product. Volatiles were partially removed and the oily product was purified by NPLC (eluting with 0-30% EtOAc in heptane, silica) to provide Intermediate 33. LCMS Method E: Rt=1.49 min, [M+H+H₂O]⁺=886.5.

### Intermediate 34: 15-(12-(benzyloxy)-12-oxododecyl)-15-((benzyloxy)carbonyl)-3,16-dioxo-1-phenyl-2,20,23,26,29,32,35,38,41,44,47,50,53,56,59,62,65,68,71,74,77,80,83,86,89-pentacosaoxa-17-azadononacontan-92-oic acid

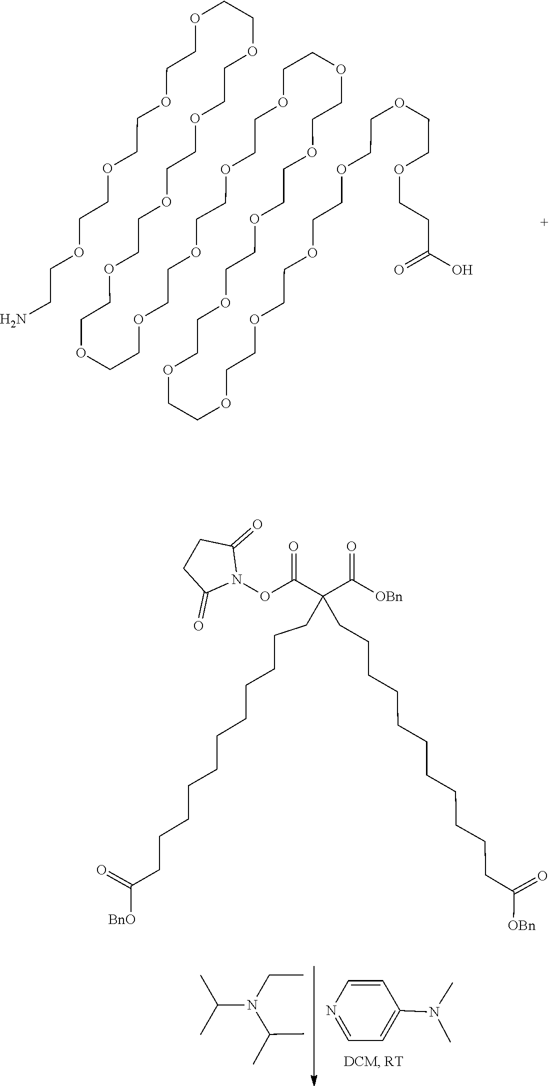

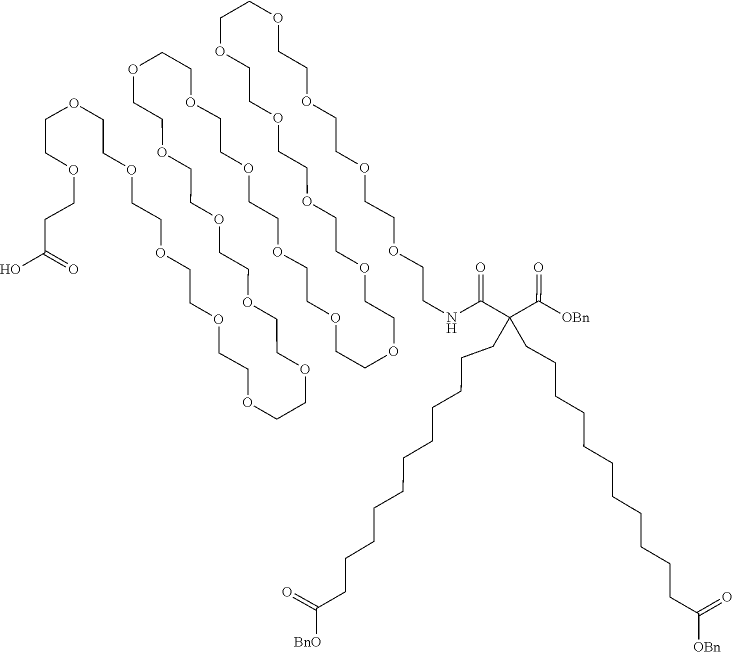 [SMILES: O=C(O)CCOCCOCCOCCOCCOCCOCCOCCOCCOCCOCCOCCOCCOCCOCCOCCOCCOCCOCCOCCOCCOCCOCCOCCOCCNC(=O)C(CCCCCCCCCCCC(=O)OCc1ccccc1)(CCCCCCCCCCCC(=O)OCc1ccccc1)C(=O)OCc1ccccc1]

Intermediate 33 (127.6 mg, 0.147 mmol) was treated with 1-amino-3,6,9,12,15,18,21,24,27,30,33,36,39,42,45,48,51,54,57,60,63,66,69,72-tetracosaoxapentaheptacontan-75-oic acid (Biopharm, 168 mg, 0.147 mmol), DIPEA (38.5 μL, 0.220 mmol) and DMAP (1.8 mg, 0.0015 mmol). After 16 h, the reaction was essentially complete. Volatiles were removed and the residue was purified by NPLC (eluting with 0-15% MeOH in DCM, silica). Fractions containing product were combined and concentrated to provide Intermediate 34. LCMS Method E: Rt=1.41 min, [M+2H]²⁺=951.6.

### Intermediate 35: 77,88-dibenzyl 1-(2,5-dioxopyrrolidin-1-yl) 77-(12-(benzyloxy)-12-oxododecyl)-76-oxo-3,6,9,12,15,18,21,24,27,30,33,36,39,42,45,48,51,54,57,60,63,66,69,72-tetracosaoxa-75-azaoctaoctacontane-1,77,88-tricarboxylate

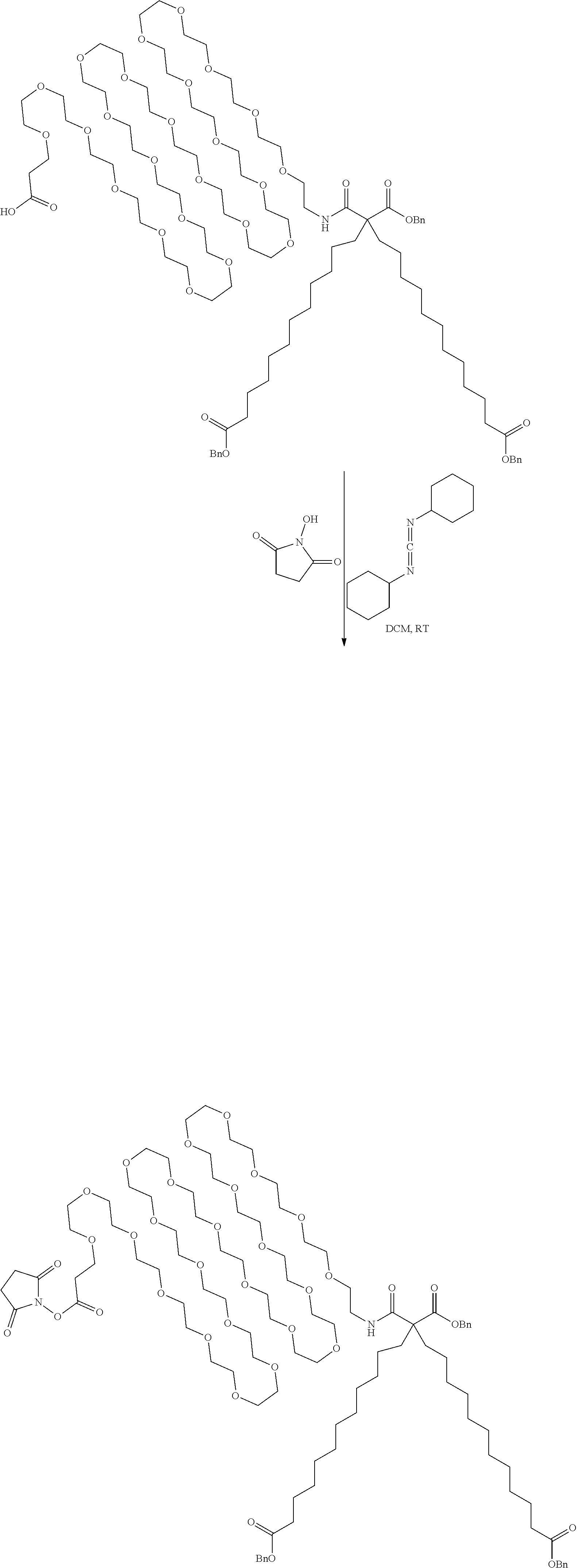

Intermediate 34 (194 mg, 0.102 mmol) was dissolved in 1 mL DCM and treated with 1-hydroxypyrrolidine-2,5-dione (11.75 mg, 0.102 mmol) and 1M DCC in DCM (Aldrich, 0.107 mL, 0.107 mmol). After 15 min, the precipitation of DCU was observed. After 16 h, the reaction was complete as indicated by LC/MS. The volatiles were removed to yield an oily residue. This material was purified by NPLC (eluting with 0-15% MeOH in DCM, silica, ELSD detection). Fractions containing product were combined and concentrated to provide the desired product, Intermediate 35. LCMS Method E: Rt=1.40 min. [M+2H+H₂O]²⁺=1008.2.

### Intermediate 36: 12-((75-((2,5-dioxopyrrolidin-1-yl)oxy)-75-oxo-3,6,9,12,15,18,21,24,27,30,33,36,39,42,45,48,51,54,57,60,63,66,69,72-tetracosaoxapentaheptacontyl)carbamoyl)tricosane-1,12,23-tricarboxylic acid

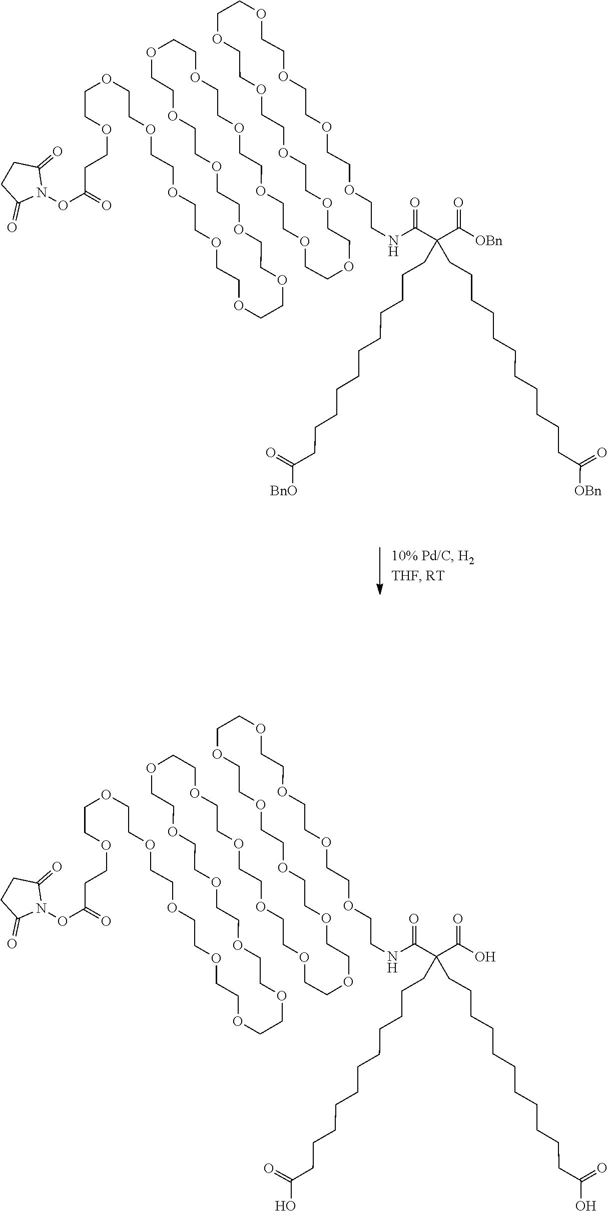 [SMILES: O=C(CCCCCCCCCCCC(CCCCCCCCCCCC(=O)OCc1ccccc1)(C(=O)NCCOCCOCCOCCOCCOCCOCCOCCOCCOCCOCCOCCOCCOCCOCCOCCOCCOCCOCCOCCOCCOCCOCCOCCOCCC(=O)ON1C(=O)CCC1=O)C(=O)OCc1ccccc1)OCc1ccccc1 ; O=C(O)CCCCCCCCCCCC(CCCCCCCCCCCC(=O)O)(C(=O)O)C(=O)NCCOCCOCCOCCOCCOCCOCCOCCOCCOCCOCCOCCOCCOCCOCCOCCOCCOCCOCCOCCOCCOCCOCCOCCOCCC(=O)ON1C(=O)CCC1=O]

Intermediate 35 (130 mg, 0.065 mmol) was dissolved in THF (2 mL) and the resulting mixture was purged three times with nitrogen. 10% palladium on carbon (36.4 mg, 0.033 mmol) was carefully added and the atmosphere was evacuated and then replaced with hydrogen from a balloon reservoir. The reaction was completed 16 h later as indicated by LC/MS analysis (ELSD detection). Purification was accomplished by NPLC (eluting with MeOH in DCM, silica, 0-20%) and fractions containing desired product were combined and concentrated to provide Intermediate 36. LCMS Method E: Rt=0.64 min, [M+2H]²⁺=864.0.

### Intermediate 37: Benzyl 14-bromotetradecanoates

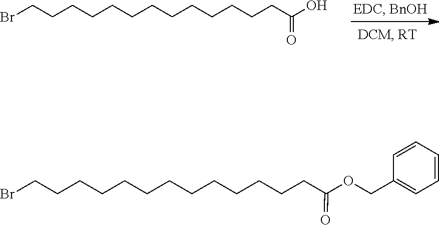 [SMILES: O=C(O)CCCCCCCCCCCCCBr ; O=C(CCCCCCCCCCCCCBr)OCc1ccccc1]

14-bromotetradecanoic acid (1.00 μm, 3.25 mmol), benzyl alcohol (677 μL, 6.51 mmol) and EDC-HCl (936 mg, 4.89 mmol) were combined in DCM (11 mL). To this solution was added DMAP (19.9 mg, 0.163 mmol) in a single portion and the resulting mixture was allowed to stir overnight. After this time, the reaction was complete as indicated by LC/MS analysis.

Volatiles were removed and the resulting residue purified by NPLC (eluting with 0-15% EtOAc in heptane, silica). Fractions containing product were combined and concentrated to provide desired Intermediate 37. ¹H NMR (400 MHz, Chloroform-d) δ 7.33-7.22 (m, 5H), 5.04 (s, 2H), 3.34 (t, J=6.9 Hz, 2H), 2.28 (t, J=7.6 Hz, 2H), 1.78 (p, 2H), 1.58 (p, J=7.3 Hz, 2H), 1.39-1.31 (m, 2H), 1.25-1.16 (m, 16H).

### Intermediate 38: 1,14,27-tribenzyl 14-(tert-butyl) heptacosane-1,14,14,27-tetracarboxylate, 1,14-dibenzyl 1-(tert-butyl) tetradecane-1,1,14-tricarboxylate

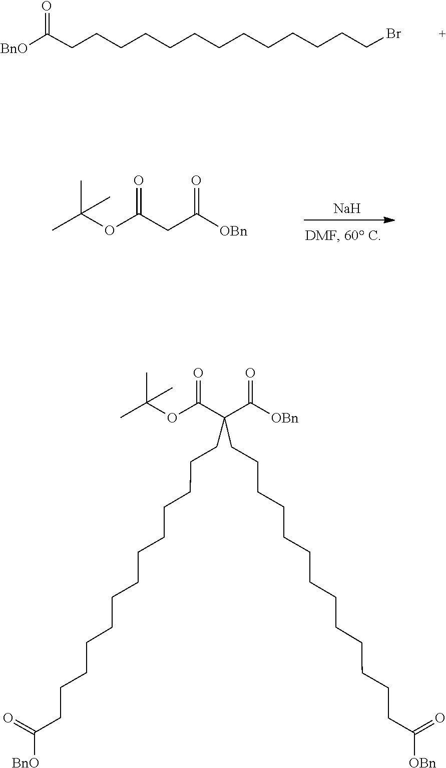

Intermediate 37 (713 mg, 1.793 mmol), benzyl tert-butyl malonate (187 mg, 0.747 mmol) and sodium hydride 60% in oil (65.7 mg, 1.644 mmol) were combined in anhydrous DMF (8 mL) and stirred at 60° C. overnight under a nitrogen atmosphere in an oven dried round bottom flask. After 24 h, LC/MS analysis indicated the mono and bis alkylated malonate products were present. The reaction mixture was treated with additional benzyl 14-bromotetradecanoate (229.2 mg, 0.57 mmol) and 60% sodium hydride in oil (45 mg, 1.13 mmol). After 16 h, the reaction was essentially complete as indicated by LC/MS. The reaction mixture was carefully partitioned between 10 mL water and 10 mL EtOAc. The aqueous phase was washed with 10 ml EtOAc. The organic phases were combined, washed with brine, dried with anhydrous sodium sulfate, and concentrated by rotovap. The crude product was purified by NPLC (eluting with 0-35% EtOAc in heptane, silica, ELSD detection). Excess starting material bromide eluted first quickly and mono alkylated product eluted second followed by desired product. Fractions containing product were combined and concentrated to provide Intermediate 38 as a clear viscous oil. LCMS Method E: Rt=1.87 min, [M+Na]⁺=905.7.

### Intermediate 39: 16-(benzyloxy)-2-(14-(benzyloxy)-14-oxotetradecyl)-2-((benzyloxy)carbonyl)-16-oxohexadecanoic acid

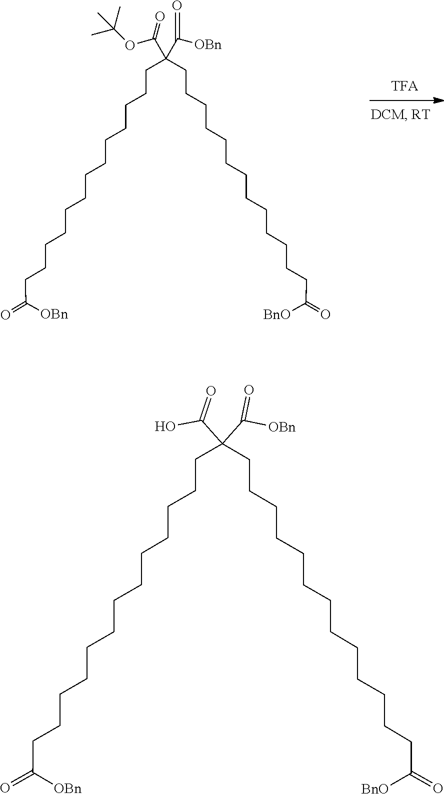 [SMILES: CC(C)(C)OC(=O)C(CCCCCCCCCCCCCC(=O)OCc1ccccc1)(CCCCCCCCCCCCCC(=O)OCc1ccccc1)C(=O)OCc1ccccc1 ; O=C(CCCCCCCCCCCCCC(CCCCCCCCCCCCCC(=O)OCc1ccccc1)(C(=O)O)C(=O)OCc1ccccc1)OCc1ccccc1]

Intermediate 38 (290.4 mg, 0.329 mmol) was dissolved into DCM (3 mL) and then treated with TFA (0.25 mL, 3.29 mmol). After 16 h, the reaction was complete as indicated by LC/MS ELSD signal. Volatiles were removed and the resulting residue was purified by NPLC (eluting with 0-5% MeOH in DCM, silica). Fractions containing product were combined and concentrated to provide Intermediate 39 as a clear oil. LCMS Method E: Rt=1.65 min, [M+H]⁺=828.1.

### Intermediate 40: 1,14,27-tribenzyl 14-(2,5-dioxopyrrolidin-1-yl) heptacosane-1,14,14,27-tetracarboxylate

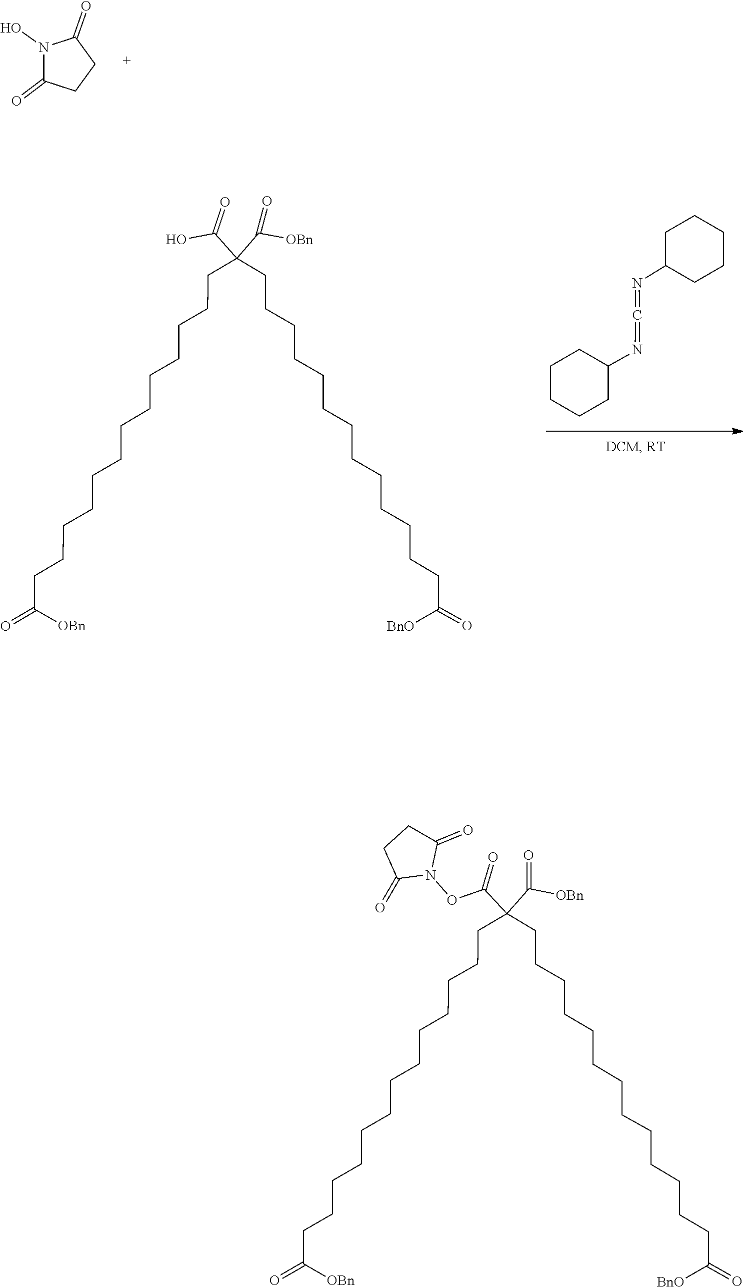 [SMILES: O=C1CCC(=O)N1O ; O=C(CCCCCCCCCCCCCC(CCCCCCCCCCCCCC(=O)OCc1ccccc1)(C(=O)O)C(=O)OCc1ccccc1)OCc1ccccc1 ; C(=NC1CCCCC1)=NC1CCCCC1 ; O=C(CCCCCCCCCCCCCC(CCCCCCCCCCCCCC(=O)OCc1ccccc1)(C(=O)OCc1ccccc1)C(=O)ON1C(=O)CCC1=O)OCc1ccccc1]

Intermediate 39 (170.4 mg, 0.206 mmol) was dissolved in DCM (2 mL) along with 1-hydroxypyrrolidine-2,5-dione (35.6 mg, 0.309 mmol) and 1M DCC in DCM (Aldrich, 212 μL, 0.212 mmol). After 10 min, the precipitation of DCU was observed. The reaction mixture was allowed to stir overnight whereupon LC/MS indicates complete conversion to product. Volatiles were partially removed and the oily product was purified by NPLC (eluting with 0-40% EtOH in heptane, silica), to provide Intermediate 40. LCMS Method E: Rt=1.69 min, [M+H]⁺=924.4.

### Intermediate 41: 17-(14-(benzyloxy)-14-oxotetradecyl)-17-((benzyloxy)carbonyl)-3,18-dioxo-1-phenyl-2,22,25,28,31,34,37,40,43,46,49,52,55,58,61,64,67,70,73,76,79,82,85,88,91-pentacosaoxa-19-azatetranonacontan-94-oic acid

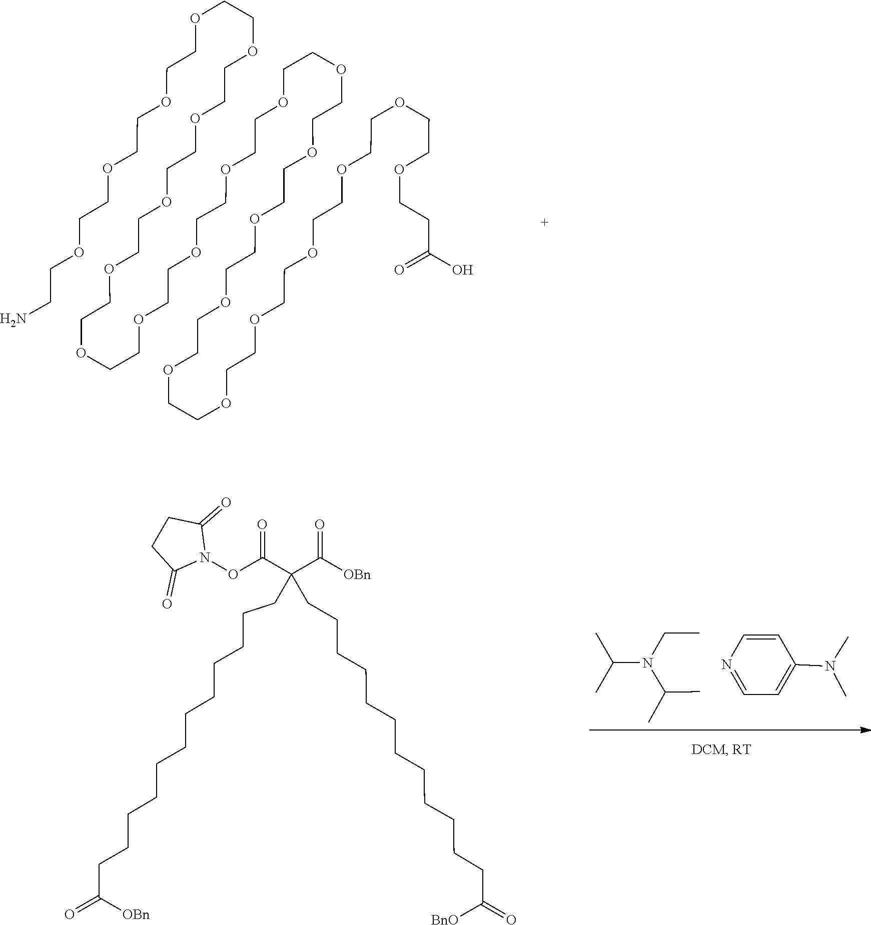

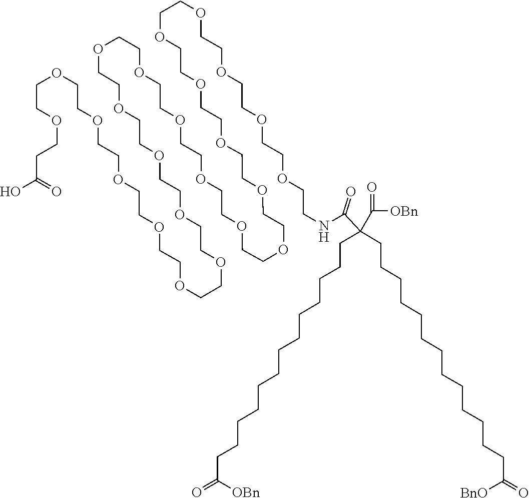 [SMILES: O=C(O)CCOCCOCCOCCOCCOCCOCCOCCOCCOCCOCCOCCOCCOCCOCCOCCOCCOCCOCCOCCOCCOCCOCCOCCOCCNC(=O)C(CCCCCCCCCCCCCC(=O)OCc1ccccc1)(CCCCCCCCCCCCCC(=O)OCc1ccccc1)C(=O)OCc1ccccc1]

Intermediate 40 (119.8 mg, 0.130 mmol) was treated with 1-amino-3,6,9,12,15,18,21,24,27,30,33,36,39,42,45,48,51,54,57,60,63,66,69,72-tetracosaoxapentaheptacontan-75-oic acid (149 mg, 0.130 mmol), DIPEA (34.0 μL, 0.194 mmol) and DMAP (1.584 mg, 0.0013 mmol) in a solution of 1.3 mL DCM. After 16 h, the reaction was essentially complete. Volatiles were removed and the residue purified by NPLC (eluting with 0-65% MeOH in DCM, silica). Fractions containing product were combined and concentrated to provide Intermediate 41. LCMS Method E: Rt=1.56 min. [M+2H]²⁺=978.9.

### Intermediate 42: 77,90-dibenzyl 1-(2,5-dioxopyrrolidin-1-yl) 77-(14-(benzyloxy)-14-oxotetradecyl)-76-oxo-3,6,9,12,15,18,21,24,27,30,33,36,39,42,45,48,51,54,57,60,63,66,69,72-tetracosaoxa-75-azanonacontane-1,77,90-tricarboxylate

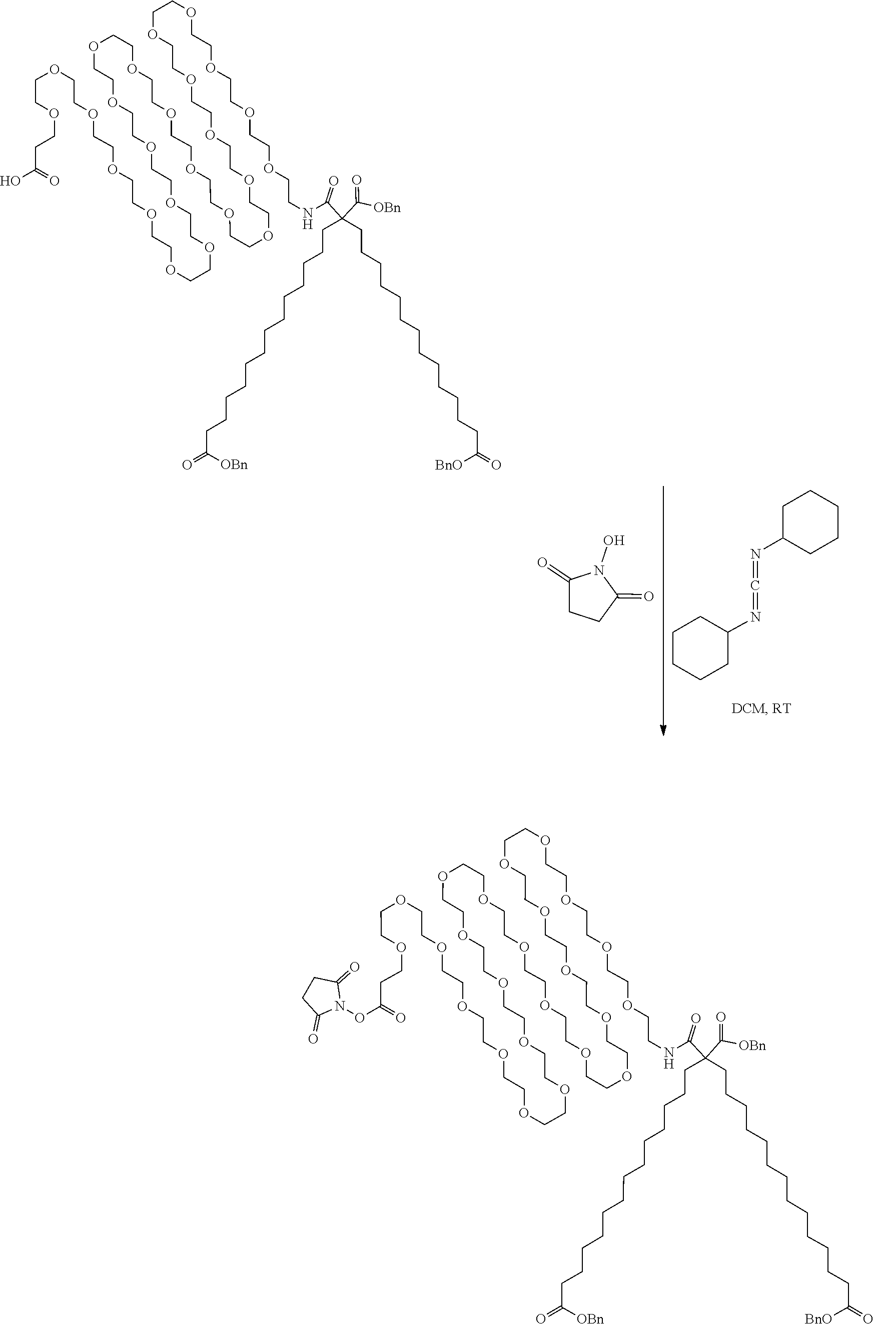 [SMILES: O=C(O)CCOCCOCCOCCOCCOCCOCCOCCOCCOCCOCCOCCOCCOCCOCCOCCOCCOCCOCCOCCOCCOCCOCCOCCOCCNC(=O)C(CCCCCCCCCCCCCC(=O)OCc1ccccc1)(CCCCCCCCCCCCCC(=O)OCc1ccccc1)C(=O)OCc1ccccc1 ; O=C1CCC(=O)N1O ; C(=NC1CCCCC1)=NC1CCCCC1 ; O=C(CCCCCCCCCCCCCC(CCCCCCCCCCCCCC(=O)OCc1ccccc1)(C(=O)NCCOCCOCCOCCOCCOCCOCCOCCOCCOCCOCCOCCOCCOCCOCCOCCOCCOCCOCCOCCOCCOCCOCCOCCOCCC(=O)ON1C(=O)CCC1=O)C(=O)OCc1ccccc1)OCc1ccccc1]

Intermediate 41 (144.2 mg, 0.074 mmol) was dissolved into 700 μL DCM and treated with 1-hydroxypyrrolidine-2,5-dione (12.73 mg, 0.111 mmol) and 1 M DCC (dicyclohexylmethanediimine) in DCM (Aldrich, 0.077 mL, 0.077 mmol). After 15 min, the precipitation of DCU was observed. After 16 h, the reaction was complete as indicated by LC/MS. The volatiles were removed to yield an oily residue. This material was purified by NPLC with ELSD detection (eluting with 0-25% MeOH in DCM, silica). Fractions containing product were combined and concentrated to provide the desired product, Intermediate 42.

LCMS Method E: Rt=1.55 min, [M+2H+H₂O]²⁺=1036.2/Intermediate 43: 14-((75-((2,5-dioxopyrrolidin-1-yl)oxy)-75-oxo-3,6,9,12,15,18,21,24,27,30,33,36,39,42,45,48,51,54,57,60,63,66,69,72-tetracosaoxapentaheptacontyl)carbamoyl)heptacosane-1,14,27-tricarboxylic acid

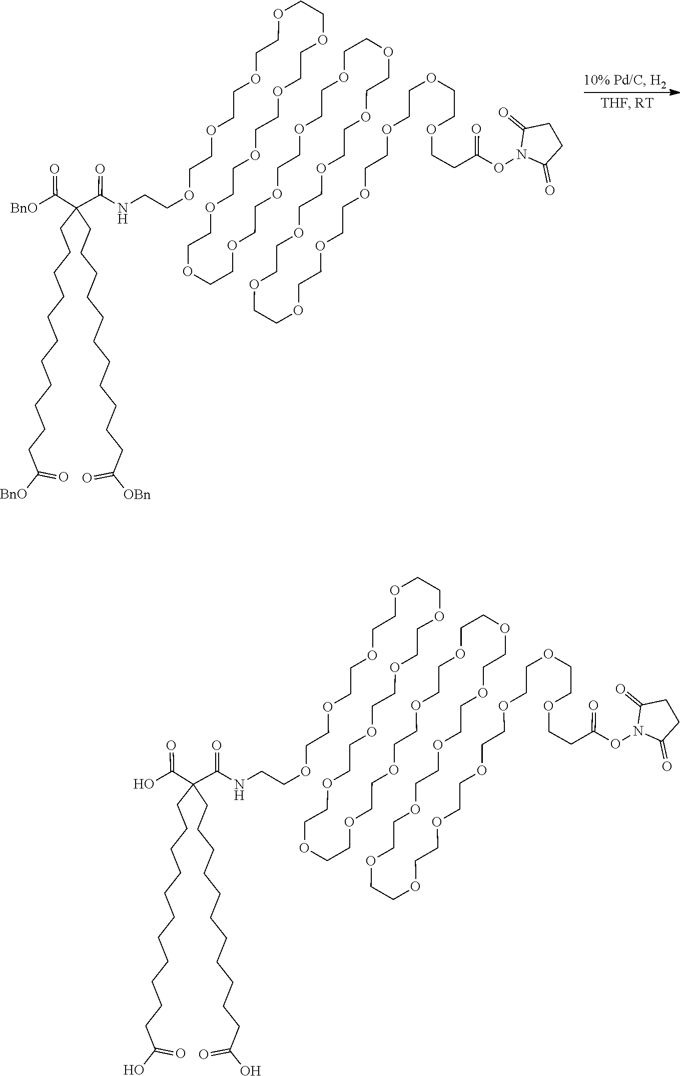 [SMILES: O=C(CCCCCCCCCCCC(CCCCCCCCCCCC(=O)OCc1ccccc1)(C(=O)NCCOCCOCCOCCOCCOCCOCCOCCOCCOCCOCCOCCOCCOCCOCCOCCOCCOCCOCCOCCOCCOCCOCCOCCOCCC(=O)ON1C(=O)CCC1=O)C(=O)OCc1ccccc1)OCc1ccccc1 ; O=C(O)CCCCCCCCCCCC(CCCCCCCCCCCC(=O)O)(C(=O)O)C(=O)NCCOCCOCCOCCOCCOCCOCCOCCOCCOCCOCCOCCOCCOCCOCCOCCOCCOCCOCCOCCOCCOCCOCCOCCOCCC(=O)ON1C(=O)CCC1=O]

Intermediate 42 (118.8 mg, 0.058 mmol) was dissolved into 3.75 mL THF in a round bottom flask with a stir bar. The resulting mixture was purged three times with nitrogen and then 30.8 mg (0.029 mmol) 10% Pd/C was added. The atmosphere was evacuated and replaced with hydrogen from a balloon. The reaction was completed in 16 h. The reaction mixture was diluted with 10 mL DCM, filtered through Celite® and the filtrate was concentrated to dryness. Purification by done by NPLC (eluting with MeOH in DCM, silica, 0-20%) and fractions containing product were combined and concentrated to provide Intermediate 43. LCMS Method E: Rt=0.86 min, [M+2H]²⁺=892.0.

### Intermediate 44: Benzyl 15-bromopentadecanoate

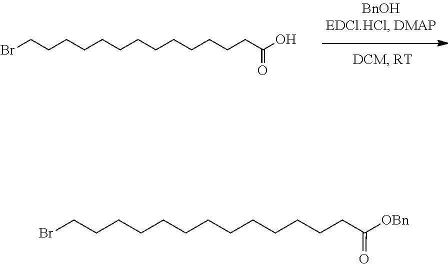 [SMILES: O=C(O)CCCCCCCCCCCCCBr ; O=C(CCCCCCCCCCCCCBr)OCc1ccccc1]

15-bromopentadecanoic acid (1949 mg, 6.07 mmol), benzyl alcohol (984 mg, 9.10 mmol) and EDCI-HCl (1744 mg, 9.10 mmol) were combined in 24 mL DCM. To this solution was added DMAP (37.1 mg, 0.303 mmol) in a single portion and the resulting mixture was allowed to stir for 32 h. After 32 h, the reaction was essentially complete. The reaction mixture was partitioned between water and DCM. The organic phase was washed with brine, dried with anhydrous sodium sulfate, and filtered. Volatiles were removed and the resulting residue purified by NPLC (eluting with 0-15% EtOAc in heptane, silica, ELSD detection). Fractions containing product were combined and concentrated to provide Intermediate 44. ¹H NMR (400 MHz, Chloroform-d) δ 7.29-7.22 (m, 5H), 5.02 (s, 2H), 3.31 (t, J=6.9 Hz, 2H), 2.26 (t, J=7.6 Hz, 2H), 1.76 (p, 2H), 1.54 (p, J=7.3 Hz, 2H), 1.36-1.30 (m, 2H), 1.22-1.16 (m, 18H).

### Intermediate 45: 1,15,29-tribenzyl 15-(tert-butyl) nonacosane-1,15,15,29-tetracarboxylate, 1,15-dibenzyl 1-(tert-butyl) pentadecane-1,1,15-tricarboxylate

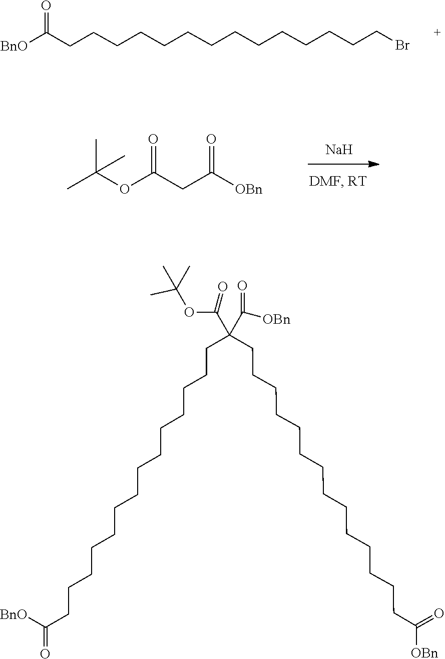

Intermediate 44 (1013 mg, 2.461 mmol), benzyl tert-butyl malonate (280 mg, 1.119 mmol) and sodium hydride 60% in oil (98 mg, 2.461 mmol) were combined in anhydrous DMF (5.6 mL) and stirred at RT overnight under a nitrogen atmosphere in an oven dried round bottom flask. The reaction mixture was then poured carefully into 10 mL water and extracted three times with 10 mL EtOAc. The organic phases were combined, dried with brine and anhydrous sodium sulfate, filtered, and concentrated. Product was purified by NPLC (eluting with 0-60% EtOAc in heptane, silica, ELSD detection). Fractions containing product were combined and concentrated to provide Intermediate 45 as a clear viscous oil. LCMS Method H: Rt=4.23 min, [M+H+H₂O]⁺=928.9.

### Intermediate 46: 17-(benzyloxy)-2-(15-(benzyloxy)-15-oxopentadecyl)-2-((benzyloxy)carbonyl)-17-oxoheptadecanoic acid

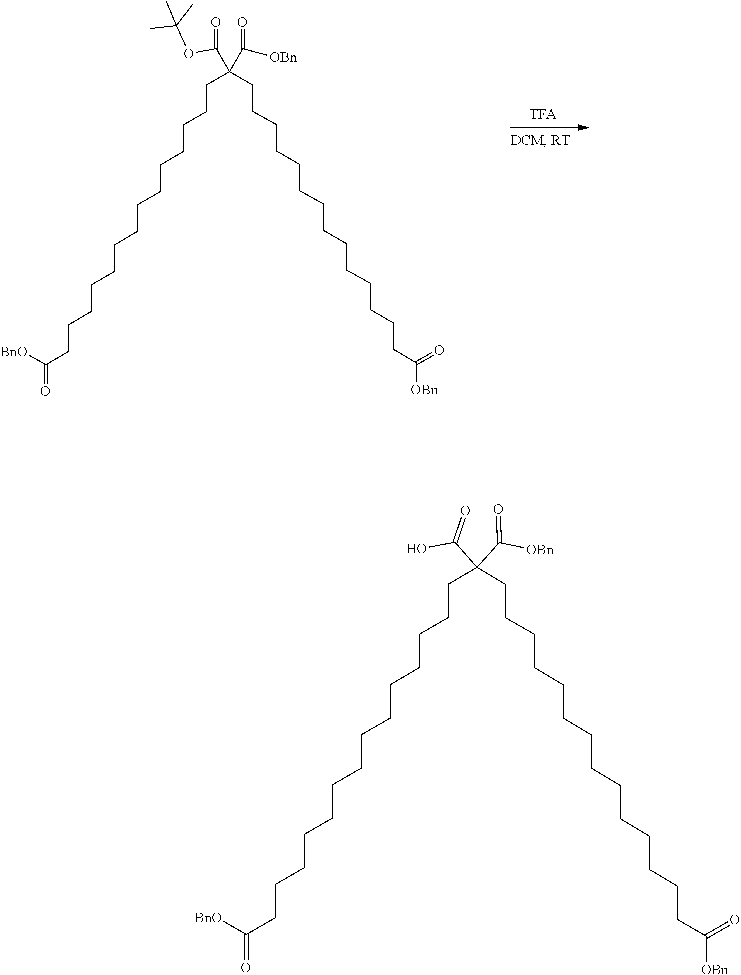 [SMILES: CC(C)(C)OC(=O)C(CCCCCCCCCCCCCCC(=O)OCc1ccccc1)(CCCCCCCCCCCCCCC(=O)OCc1ccccc1)C(=O)OCc1ccccc1 ; O=C(CCCCCCCCCCCCCCC(CCCCCCCCCCCCCCC(=O)OCc1ccccc1)(C(=O)O)C(=O)OCc1ccccc1)OCc1ccccc1]

Intermediate 45 (750 mg, 0.823 mmol) was dissolved into DCM (8.23 mL) and treated with TFA (634 μL, 8.23 mmol). After 16 h. the reaction was partially complete. The reaction mixture was left stirring for a week and after this time was essentially complete. The resulting oily residue was purified by NPLC (eluting with 0-25% EtOAc in heptanes, silica) with ELSD detection. Fractions containing product were concentrated to give Intermediate 46. LCMS Method H: Rt=3.70 min, [M+H+H₂O]⁺=873.2.

### Intermediate 47: 1,15,29-tribenzyl 15-(2,5-dioxopyrrolidin-1-yl) nonacosane-1,15,15,29-tetracarboxylate

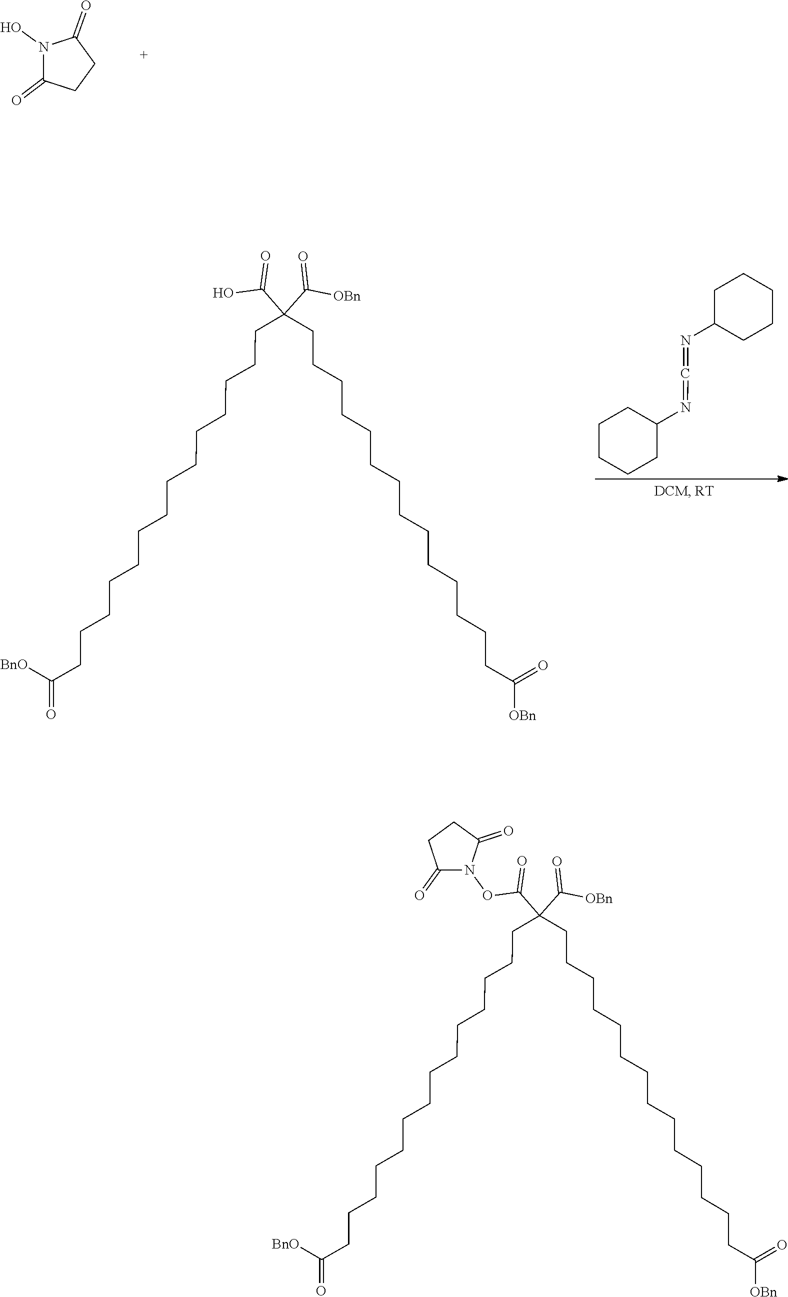 [SMILES: O=C1CCC(=O)N1O ; "+" ; O=C(CCCCCCCCCCCCCCC(CCCCCCCCCCCCCCC(=O)OCc1ccccc1)(C(=O)O)C(=O)OCc1ccccc1)OCc1ccccc1 ; C(=NC1CCCCC1)=NC1CCCCC1 ; O=C(CCCCCCCCCCCCCCC(CCCCCCCCCCCCCCC(=O)OCc1ccccc1)(C(=O)OCc1ccccc1)C(=O)ON1C(=O)CCC1=O)OCc1ccccc1]

Intermediate 46 (435 mg, 0.509 mmol) and 1-hydroxypyrrolidine-2,5-dione (64.4 mg, 0.560 mmol) were suspended into 5 mL anhydrous DCM (5 mL) and 1 M DCC in DCM (Aldrich, 534 μL, 0.534 mmol) was added. The resulting mixture was stirred at RT. After 15 min, a fine precipitate (ppt) formed suggesting the formation of DCU. After 16 h, the reaction was complete as indicated by LC/MS. The volatiles were removed by evaporation. The oily residue was purified by NPLC (eluting with 0-10% MeOH in DCM, silica, ELSD detection). Fractions containing product were combined and concentrated to provide Intermediate 47. LCMS Method I: Rt=3.62 min. [M+H₂O+H]⁺=970.1.

### Intermediate 48: 18-(15-(benzyloxy)-15-oxopentadecyl)-18-((benzyloxy)carbonyl)-3,19-dioxo-1-phenyl-2,23,26,29,32,35,38,41,44,47,50,53,56,59,62,65,68,71,74,77,80,83,86,89,92-pentacosaoxa-20-azapentanonacontan-95-oic acid

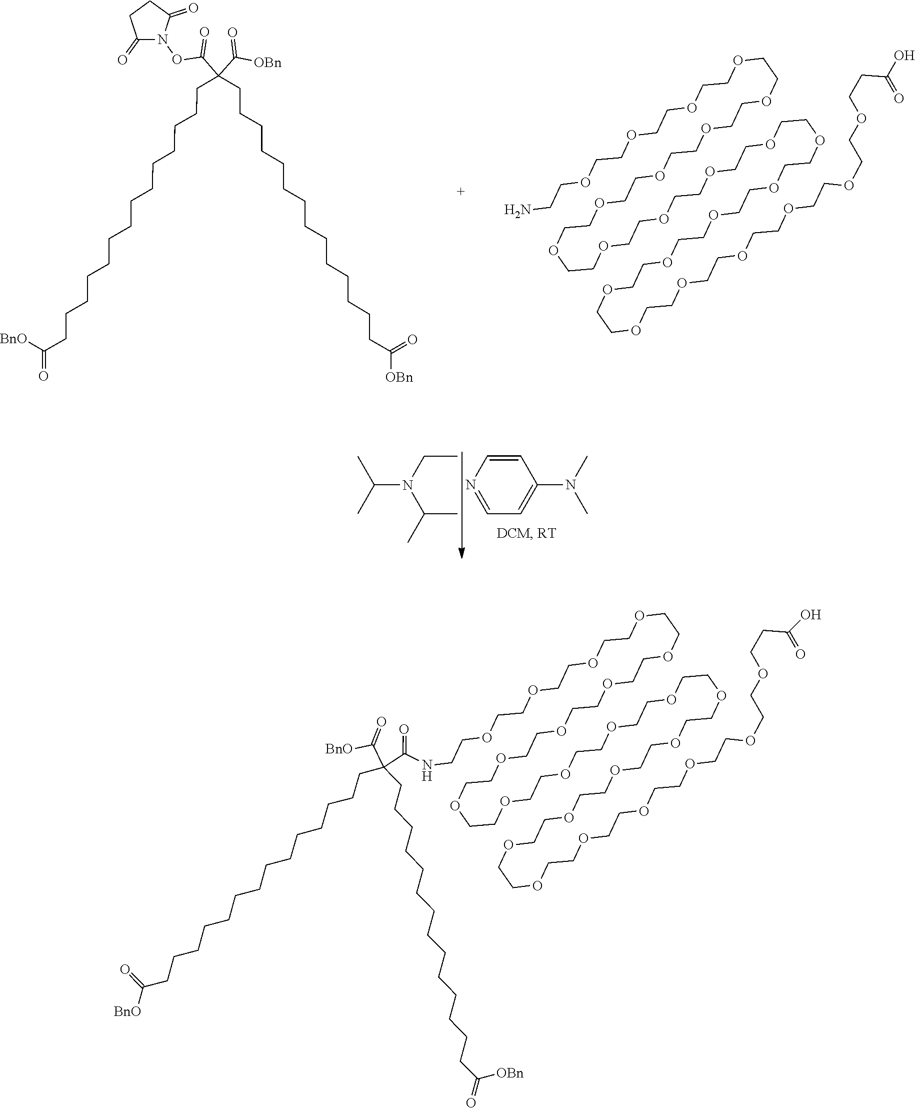

Intermediate 47 (143 mg, 150 mmol) was dissolved into 1.5 mL DCM in a 2 dram screw cap vial along with 1-amino-3,6,9,12,15,18,21,24,27,30,33,36,39,42,45,48,51,54,57,60,63,66,69,72-tetracosaoxapentaheptacontan-75-oic acid (198 mg, 0.173 mmol), DIPEA (48.5 μL, 0.375 mmol) and DMAP (2 mg, 0.109 mmol). The resulting mixture was allowed to stir overnight. Then the volatiles were removed and the resulting residue was purified via NPLC (eluting with 0-10% MeOH in DCM, silica). Fractions containing product were combined and concentrated to provide Intermediate 48 as clear semi-solid. LCMS Method I: Rt=2.53 min, [M+2H+2H₂O]²⁺=1010.1.

### Intermediate 49: 77,91-dibenzyl 1-(2,5-dioxopyrrolidin-1-yl) 77-(15-(benzyloxy)-15-oxopentadecyl)-76-oxo-3,6,9,12,15,18,21,24,27,30,33,36,39,42,45,48,51,54,57,60,63,66,69,72-tetracosaoxa-75-azahennonacontane-1,77,91-tricarboxylate

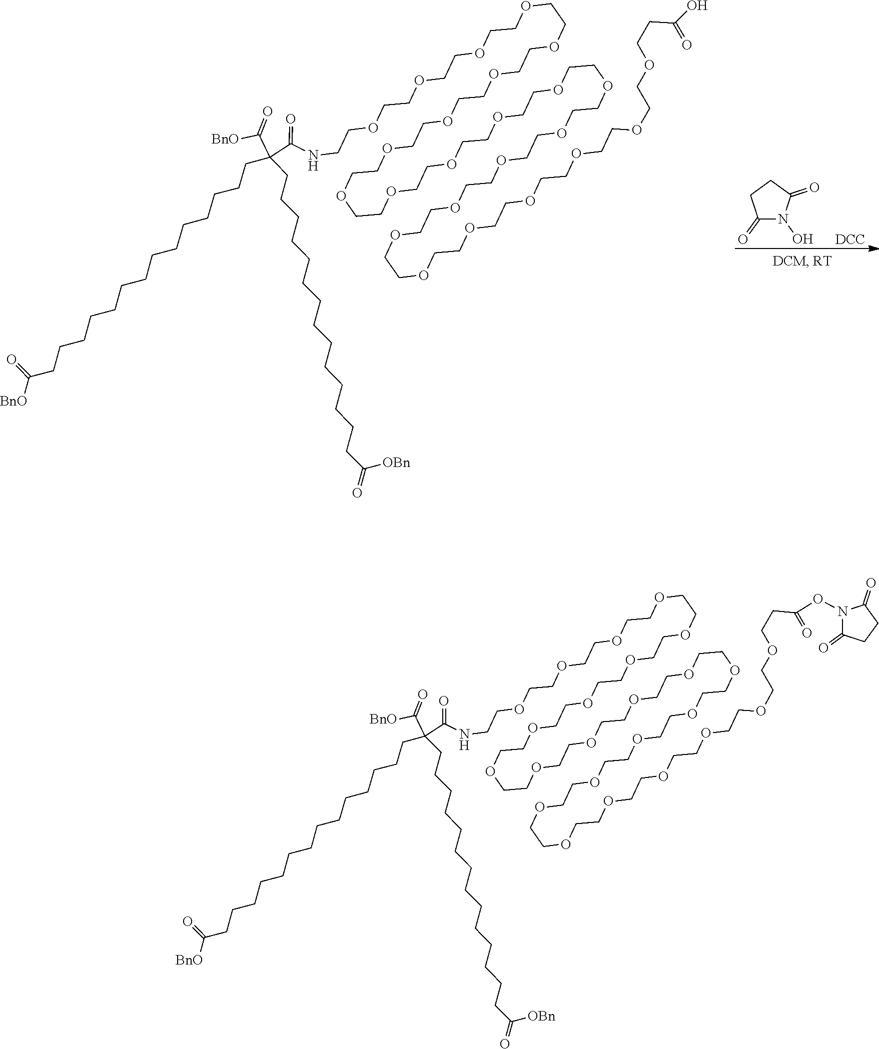

Intermediate 48 (210 mg, 0.106 mmol) and 1-hydroxypyrrolidine-2,5-dione (13.4 mg, 0.116 mmol) were suspended into 1 mL anhydrous DCM with stirring in an oven dried 10 mL round bottom flask (RBF). To this mixture was added 1 M DCC in DCM (Aldrich, 116 μL, 0.116 mmol). After 16 h, the reaction was complete as indicated by LC/MS. Volatiles were removed and the resulting residue purified by NPLC (eluting with 0-15% MeOH in DCM, silica, ELSD detection). Fractions containing desired product were combined and concentrated to yield Intermediate 49 as a waxy solid. LCMS Method H: Rt=3.48 min, [M+H₂O+2H]²⁺=1049.9.

### Intermediate 50: 15-((75-((2,5-dioxopyrrolidin-1-yl)oxy)-75-oxo-3,6,9,12,15,18,21,24,27,30,33,36,39,42,45,48,51,54,57,60,63,66,69,72-tetracosaoxapentaheptacontyl)carbamoyl)nonacosane-1,15,29-tricarboxylic acid

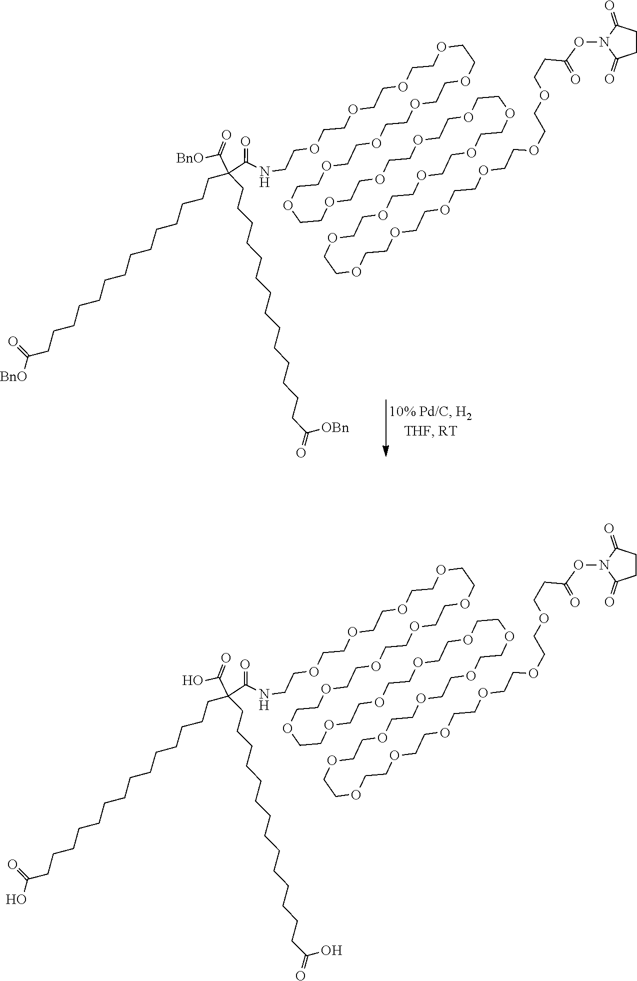 [SMILES: O=C(CCCCCCCCCCCCCCC(CCCCCCCCCCCCCCC(=O)OCc1ccccc1)(C(=O)NCCOCCOCCOCCOCCOCCOCCOCCOCCOCCOCCOCCOCCOCCOCCOCCOCCOCCOCCOCCOCCOCCOCCOCCOCCC(=O)ON1C(=O)CCC1=O)C(=O)OCc1ccccc1)OCc1ccccc1 ; O=C(O)CCCCCCCCCCCCCCC(CCCCCCCCCCCCCCC(=O)O)(C(=O)O)C(=O)NCCOCCOCCOCCOCCOCCOCCOCCOCCOCCOCCOCCOCCOCCOCCOCCOCCOCCOCCOCCOCCOCCOCCOCCOCCC(=O)ON1C(=O)CCC1=O]

Intermediate 49 (140 mg, 0.067 mmol) was dissolved in anhydrous THF (1 mL) in a round bottom flask equipped with a stir bar and the resulting mixture was purged three times with N₂. Dry 10% Pd on carbon (7 mg, 6.73 umol) and 20% Pd hydroxide on carbon (Aldrich) (5 mg, 6.73 umol) were then added and the atmosphere was evacuated and replaced with hydrogen from a balloon. The reaction mixture was allowed to stir for 16 h. The atmosphere was then evacuated and replaced with N₂. The reaction mixture was diluted with 5 mL anhydrous DCM. After filtration through Celite®, the volatiles were removed to provide Intermediate 50 as a viscous oil. LCMS Method D: Rt=1.36 min. [M+2H]2+=906.2.

### Peptide Synthesis:

The GLP1 peptides can be synthesized using standard synthetic techniques e.g. solid phase peptide synthesis techniques as mentioned in Jose Palomo RSC Adv., 2014, 4, 32658-32672; recombinant DNA techniques as described in Sambrook et al. Molecular Cloning: A Laboratory Manual, 2^{nd} Ed., Cold Spring Harbor (1989) and similar references.

### GLP-1 Analogue Synthesis: [Fmoc-His7, Aib8, Arg34]GLP-1 (7-37)

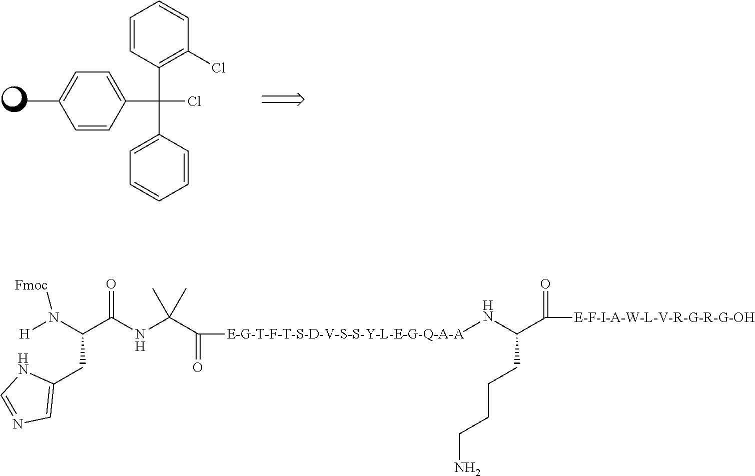

The peptide was synthesized using standard Fmoc chemistry.
1) Resin preparation: To 1-chloro-2-[chloro(diphenyl)methyl]benzene (100 mmol, 1.00 equiv) was added Fmoc-Gly-OH (50 mol, 0.50 equiv) and DIEA (400 mmol, 4.00 equiv) in DCM (4.00 mL). The resulting mixture was agitated under a nitrogen atmosphere for 2 h at 25° C. MeOH (100 mL) was then added and the mixture was agitated under an atmosphere of nitrogen for another 30 min. The resin was washed with DMF (500 mL) thrice. Then 20% piperidine in DMF (500 mL) was added and the mixture was agitated an atmosphere of nitrogen for 20 min at 25° C. The resulting mixture was filtered to provide the resin. The resin was washed with DMF (500 mL) five times and then filtered to provide the resin.
2) Coupling: a solution of DIEA (200 mmol, 4.00 equiv), Fmoc-Arg(Pbf)-OH (100 mmol, 2.00 equiv) and HBTU (95 mmol, 1.90 equiv) in DMF (300 mL) was added to the resin and agitated under an atmosphere of nitrogen for 30 min at 25° C. The resin was then washed with DMF (500 mL) thrice.
3) Deprotection: 20% piperidine in DMF (500 mL) was added to the resin and the resulting mixture was agitated under an atmosphere of nitrogen for 20 min at 25° C. The resin was washed with DMF (500 mL) five times and filtered to provide the resin.

The above coupling step 2 and the deprotection step 3 were then repeated with the further amino acids units #3 to 31 to yield the GLP-1 or GLP-1 analogue.

| Unit | | |
|---|---|---|
| # | Materials | Coupling reagents |
| 1 | Fmoc-Gly-OH(2.00 equiv) | HBTU (1.90 equiv) and DIEA (4.00 equiv) |
| 2 | Fmoc-Arg(Pbf)-OH (2.00 equiv) | HBTU (1.90 equiv) and DIEA (4.00 equiv) |
| 3 | Fmoc-Gly-OH(2.00 equiv) | HBTU (1.90 equiv) and DIEA (4.00 equiv) |
| 4 | Fmoc-Arg(Pbf)-OH (2.00 equiv) | HBTU (1.90 equiv) and DIEA (4.00 equiv) |
| 5 | Fmoc-Val-OH (2.00 equiv) | HBTU (1.90 equiv) and DIEA (4.00 equiv) |
| 6 | Fmoc-Leu-OH (2.00 equiv) | HBTU (1.90 equiv) and DIEA (4.00 equiv) |
| 7 | Fmoc-Trp(Boc)—OH (2.00 equiv) | HBTU (1.90 equiv) and DIEA (4.00 equiv) |
| 8 | Fmoc-Ala-OH (2.00 equiv) | HBTU (1.90 equiv) and DIEA (4.00 equiv) |
| 9 | Fmoc-Ile-OH (2.00 equiv) | HBTU (1.90 equiv) and DIEA (4.00 equiv) |
| 10 | Fmoc-Phe-OH (2.00 equiv) | HBTU (1.90 equiv) and DIEA (4.00 equiv) |
| 11 | Fmoc-Glu(Otbu)-OH (3.00 equiv) | HOAt (3.00 equiv) and DIC (3.00 equiv) |
| 11 | Fmoc-Glu(Otbu)-OH (3.00 equiv) | HOAt (3.00 equiv) and DIC (3.00 equiv) |
| 12 | Fmoc-Lys(Boc)—OH (3.00 equiv) | HOAt (3.00 equiv) and DIC (3.00 equiv) |
| 13 | Fmoc-Ala-OH (3.00 equiv) | HOAt (3.00 equiv) and DIC (3.00 equiv) |
| 14 | Fmoc-Ala-OH (3.00 equiv) | HOAt (3.00 equiv) and DIC (3.00 equiv) |
| 15 | Fmoc-Gln(Trt)-OH (3.00 equiv) | HOAt (3.00 equiv) and DIC (3.00 equiv) |
| 15 | Fmoc-Gln(Trt)-OH (3.00 equiv) | HOAt (3.00 equiv) and DIC (3.00 equiv) |
| 16 | Fmoc-Gly-OH(3.00 equiv) | HOAt (3.00 equiv) and DIC (3.00 equiv) |
| 17 | Fmoc-Glu(Otbu)-OH (3.00 equiv) | HOAt (3.00 equiv) and DIC (3.00 equiv) |
| 18 | Fmoc-Leu-OH (3.00 equiv) | HOAt (3.00 equiv) and DIC (3.00 equiv) |
| 19 | Fmoc-Tyr(tBu)-OH (3.00 equiv) | HOAt (3.00 equiv) and DIC (3.00 equiv) |
| 20 | Fmoc-Ser (tBu)-OH (3.00 equiv) | HOAt (3.00 equiv) and DIC (3.00 equiv) |
| 21 | Fmoc-Ser (tBu)-OH (3.00 equiv) | HOAt (3.00 equiv) and DIC (3.00 equiv) |
| 22 | Fmoc-Val-OH (3.00 equiv) | HOAt (3.00 equiv) and DIC (3.00 equiv) |
| 23 | Fmoc-Asp(Otbu)-OH (3.00 equiv) | HOAt (3.00 equiv) and DIC (3.00 equiv) |
| 24 | Fmoc-Ser (tBu)-OH (3.00 equiv) | HOAt (3.00 equiv) and DIC (3.00 equiv) |
| 25 | Fmoc-Thr (tBu)-OH (3.00 equiv) | HOAt (3.00 equiv) and DIC (3.00 equiv) |
| 26 | Fmoc-Phe-OH (3.00 equiv) | HOAt (3.00 equiv) and DIC (3.00 equiv) |
| 27 | Fmoc-Thr (tBu)-OH (3.00 equiv) | HOAt (3.00 equiv) and DIC (3.00 equiv) |
| 28 | Fmoc-Gly-OH(3.00 equiv) | HOAt (3.00 equiv) and DIC (3.00 equiv) |
| 29 | Fmoc-Glu(Otbu)-OH (3.00 equiv) | HOAt (3.00 equiv) and DIC (3.00 equiv) |
| 30 | Fmoc-Aib-OH (3.00 equiv) | HOAt (3.00 equiv) and DIC (3.00 equiv) |
| 31 | Fmoc-His(Trt)-OH (3.00 equiv) | HOAt (3.00 equiv) and DIC (3.00 equiv) |

The coupling reaction was monitored by ninhydrin test, and the resin was washed 5 times with DMF.

### Peptide Cleavage and Purification:

1) Cleavage buffer (92.5% TFA/2.5%3-mercaptopropionic acid/2.5% TIS/2.5% H₂O) was added to the flask containing the side chain protected peptide at RT and then stirred for 2 h.
2) The precipitated peptide was washed with cold isopropyl ether.
3) The precipitated peptide was filtered and the filter cake collected.
4) The precipitated peptide was washed with isopropyl ether two more times.
5) The crude peptide was then dried under vacuum for 2 h.

The crude peptide was purified by prep-HPLC (TFA condition; 30° C., eluting with A: 0.075% TFA in H₂O, B:CH₃CN) and purified by prep-HPLC (HOAc condition, eluting with A: 0.5% HAc in H₂O, B: ACN) to provide [Fmoc-His7, Aib8, Arg34]GLP-1-(7-37) as a white solid.

Conjugation of Fatty Acid Derivative (with Linker) to Peptide:

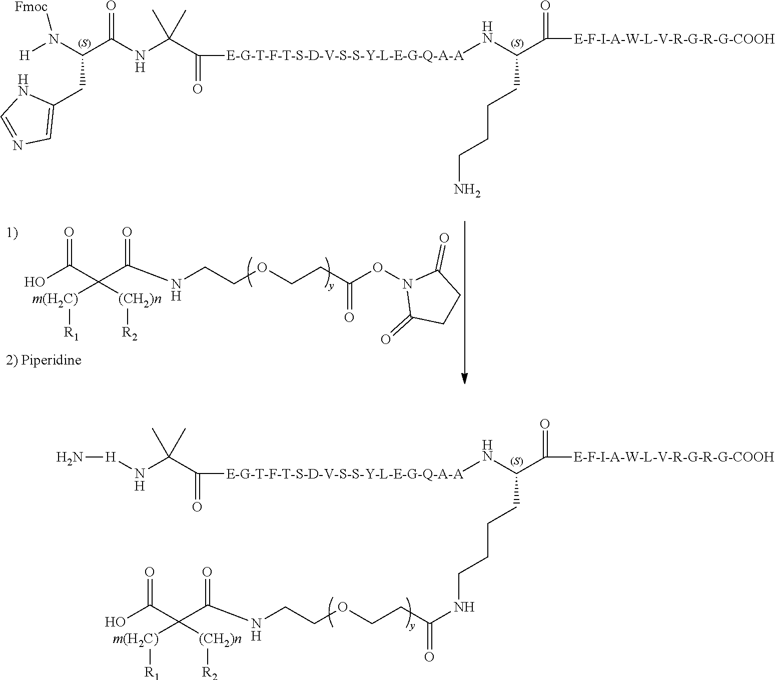

### General Procedure for Peptide Conjugation:

Method A: [Fmoc-His7, Aib8, Arg34]GLP-1-(7-37) was taken in DMA and the desired ‘fatty acid-linker conjugate’ NHS-ester was added. This mixture was allowed to stir at RT for 16-40 h. Once complete conversion was observed by LCMS analysis, 10 equiv of piperidine was added and stirring was continued for 2 h to remove the Fmoc group.

The crude product was purified by HPLC (Column: Waters XSelect C18 CSH 19×150 mm; 5 micron) eluting with 0-100% ACN in water with 0.1% TFA modifier (30 mL/min) to provide the TFA salt of the desired compound as white fluffy solid. The residual TFA was removed by taking the compound in water along with BT AG 1-XB Resin (cat #143-2446; BIO-RAD) and stirring the resulting mixture for 1 h. The mixture was then filtered and the resin was washed with acetonitrile and water. The solution was lyophilized to provide the desired compound.

Method B: [Fmoc-His7, Aib8, Arg34]GLP-1-(7-37) was taken in DMF and the desired ‘fatty acid-linker conjugate’ NHS fatty acid was added. This mixture was allowed to stir at RT for 16-40 h. Once complete conversion was observed by LCMS analysis, 10 equiv of piperidine was added and stirring was continued for an additional 2 h to remove the Fmoc group. The crude product was diluted with 0.1 N aqueous ammonium carbonate and purified by RPLC (ISCO Gold C18 150 gram column, eluting with 10-100% ACN in water, 0.1% formic acid modifier). Pure fractions containing desired product were combined and lyophilized to provide the desired compound.

### Compound 1 (Diastereomeric Mixture)

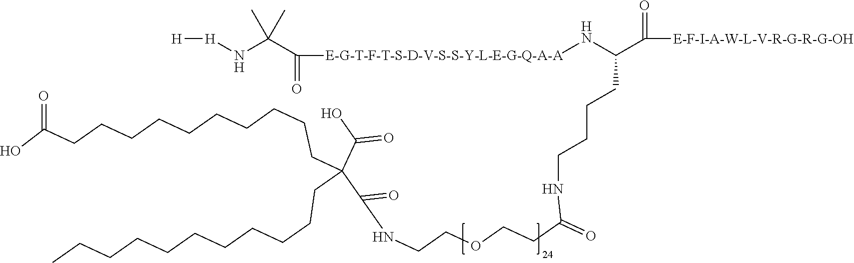

To a solution of [Fmoc-His7, Aib8, Arg34]GLP-1-(7-37) (356.6 mg, 0.099 mmol) in anhydrous DMF (9.8 mL) was added a solution of Intermediate 6 (197 mg, 0.118 mmol) in 1.5 mL anhydrous DMF. After 16 h, the reaction had gone to about 85% conversion so an additional 0.2 equiv of Intermediate 6 was added (32.9 mg in 0.5 mL anhydrous DMF). After 48 h, the reaction was complete. Then the Fmoc removal was initiated with the addition of piperidine (98 μL, 0.985 mmol, 10 equiv). After 16 h, Fmoc removal was complete, and the solvent was removed under reduced pressure. The above crude product was taken into 25 mL 0.1 N aqueous ammonium carbonate and injected (twice) on ISCO RediSep Gold C18Aq 100 Gram column (catalog no 69-2203-562) eluting with a 0-100% ACN in water as eluent with 0.1% TFA modifier. The fractions containing the product were lyophilized to provide a white fluffy powder. To scavenge for residual TFA, 700 mg of hydroxide resin (BioRad AG 1-X8) was weighed into an Eppendorf tube and rinsed 5×2 mL 1:1 ACN:H₂O, 0.1% formic acid with supernatant removed and discarded between each rinse (centrifuge to deposit resin). Rinsed resin was added to the above prepared solution of product and shaken for 1 h. Supernatant was filtered and then rinsing with 10 mL 1:1 ACN:H₂O with 0.1% formic acid. This solution was lyophilized to provide compound 1 as a white powder. LCMS Method J: Observed m/z=2474.8447 (MH₂2+), Rt: 1.16 min; Calculated mass: 4947.6750. LCMS Method K: Observed m/z=4948.7002 (MH)+, Rt: 2.31 min; Calculated mass: 4947.6750.

### Compound 2: Diastereomer 1

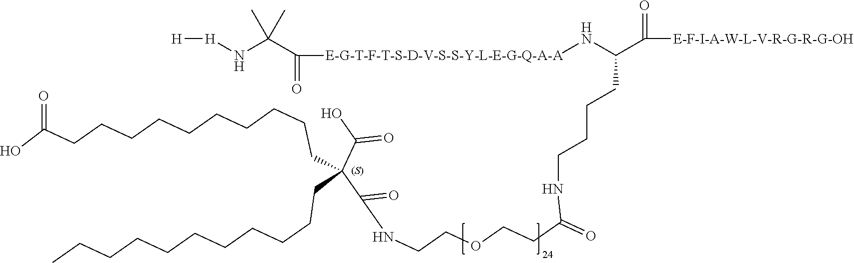

Compound 2 was synthesized using the general procedure for conjugation, method B, and using Intermediate 15A (S-enantiomer) as starting material.

LCMS Method F: Observed m/z=1238.5 (MH₄⁴⁺), Rt: 2.25 min; Calculated mass: 4947.6750.

LCMS Method K: Observed 4948.7002 (MH⁺), Rt: 2.31 min; Calculated mass: 4947.6750.

### Alternative Method for Synthesizing Compound 2:

In analogy to the reactions described for converting Intermediate 3 to 4, then to 5 and finally to Intermediate 6, the alternative method for synthesizing Compound 2 starts with Intermediate 3B (S-enantiomer), which is converted to 4B (═S-enantiomer of Intermediate 4), then to 5B (═S-enantiomer of Intermediate 5) and finally to Intermediate 6B (═S-enantiomer of Intermediate 6). Intermediate 6B and [Fmoc-His7, Aib8, Arg34]GLP-1-(7-37) are then reacted in accordance to the general procedure for conjugation to obtain Compound 2.

The absolute configuration in the fatty acid portion for Compound 2 was determined to be S by using single X-ray crystallography of a derivative of the enantiomerically pure Intermediate 3B which was used as starting material for the synthesis of Compound 2.

### Compound 3: Diastereomer 2

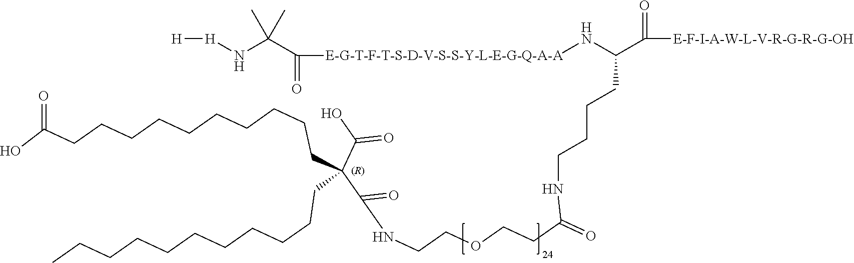

Compound 3 was synthesized using the general procedure for conjugation, method B, and using Intermediate 15B (R-enantiomer) as starting material.

LCMS Method F: Observed m/z=1238.6 (MH₄⁴⁺) Rt: 2.29 min; Calculated mass: 4947.6750.

LCMS Method K: Observed 4948.7002 (MH⁺), Rt: 2.31 min; Calculated mass: 4947.6750

### Alternative Method for Synthesizing Compound 3:

In analogy to the reactions described for converting Intermediate 3 to 4, then to 5 and finally to Intermediate 6, the alternative method for Compound 3 starts with Intermediate 3A (R-enantiomer), which is converted to 4A (═R-enantiomer of Intermediate 4), then to 5A (═R-enantiomer of Intermediate 5) and finally to Intermediate 6A (═R-enantiomer of Intermediate 6). Intermediate 6A and [Fmoc-His7, Aib8, Arg34]GLP-1-(7-37) are then reacted in accordance to the general procedure for conjugation to obtain Compound 3.

The absolute configuration in the fatty acid portion for Compound 3 was determined to be R. This was determined by single X-ray crystallography of a derivative of the enantiomerically pure Intermediate 3A.

### Compound 4:

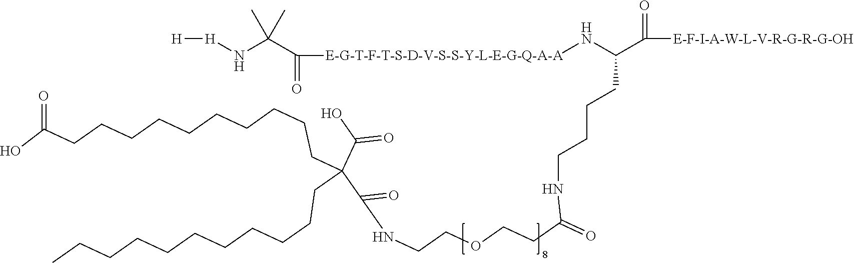

Compound 4 was synthesized using the general procedure for conjugation, method A using Intermediate 19.

LCMS Method F: Observed m/z=1061.7 (MH₄₄+), Rt: 2.26 min; Calculated mass: 4243.2556.

### Compound 5:

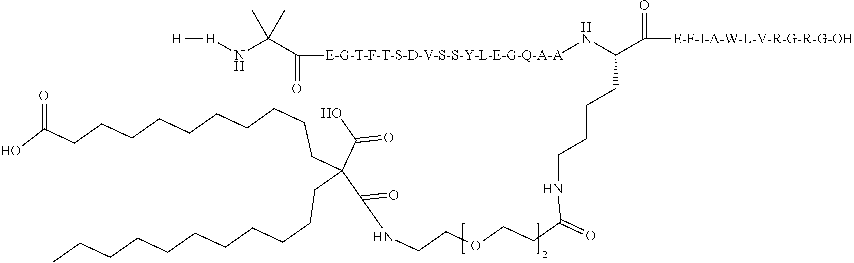

Compound 5 was synthesized using the general procedure for conjugation, method B using Intermediate 22.

LCMS Method F: Observed m/z=996.6 (MH₄⁴⁺), Rt: 2.34 min; Calculated mass: 3979.0983.

### Compound 6:

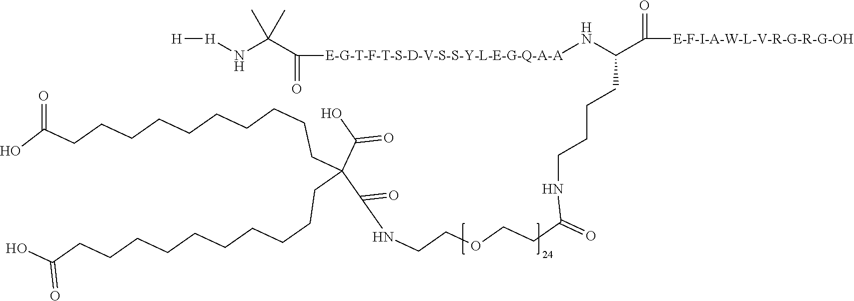

Compound 6 was synthesized using the general procedure for conjugation, method B using Intermediate 29.

LCMS Method F: Observed m/z=2490.9 (MH₂₂+), Rt: 2.09 min; Calculated mass: 4977.6495.

LCMS Method K: Observed 4978.6602 (MH⁺), Rt: 2.09 min; Calculated mass: 4977.6495.

### Compound 7:

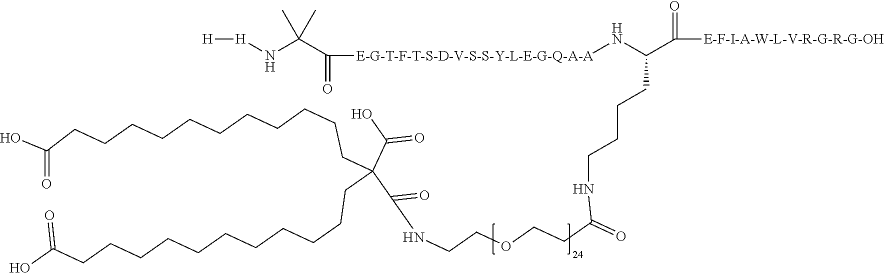

Compound 7 was synthesized using the general procedure for conjugation, method B using Intermediate 36.

LCMS Method F: Observed m/z=1253.1 (MH₄₄+), Rt: 2.13 min; Calculated mass: 5005.6805

LCMS Method K: Observed 5006.7100 (MH⁺), Rt: 2.15 min; Calculated mass: 5005.6805

### Compound 8:

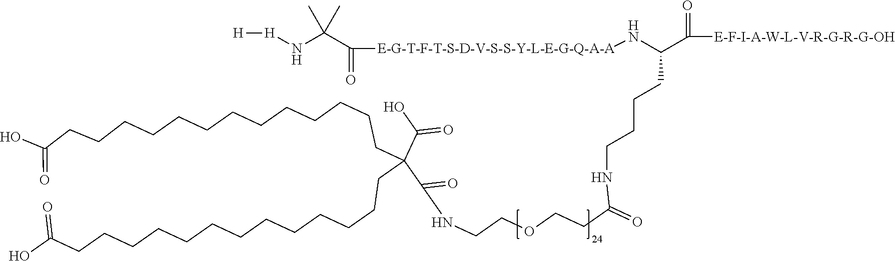

Compound 8 was synthesized using the general procedure for conjugation, method B using Intermediate 43.

LCMS Method K: Observed 5062.7700 (MH⁺), Rt: 2.30 min; Calculated mass: 5061.7431

### Compound 9:

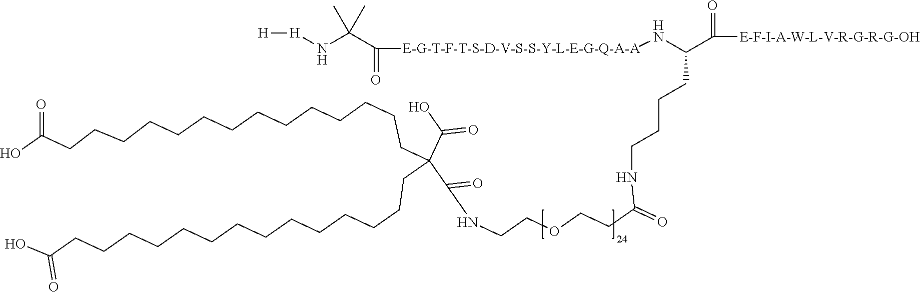

Compound 9 was synthesized using the general procedure for conjugation, method A using Intermediate 50.

LCMS Method J: Observed m/z=1274.0 (MH₄⁴⁺), Rt: 1.18 min; Calculated mass: 5089.7744.

Those skilled in the art will recognize, or be able to ascertain, using no more than routine experimentation, numerous equivalents to the specific embodiments described specifically herein. Such equivalents are intended to be encompassed in the scope of the following claims.

1. A compound of formula (I) or a pharmaceutically acceptable salt thereof: 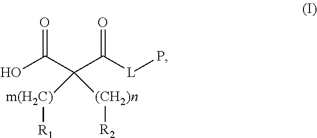
wherein:
R₁ and R₂ are independently selected from CH₃, OH, CO₂H, CH═CH₂ and C≡CH;
n and m are each an integer independently selected from 5 to 30;
L is an optional linker;
P is GLP-1 or a GLP-1 analogue.

2. A compound of formula (I) or a pharmaceutically acceptable salt thereof according to claim 1, wherein P is bound to L via an NH group.

3. A compound of formula (I) or a pharmaceutically acceptable salt thereof according to claim 1, wherein L is selected from: 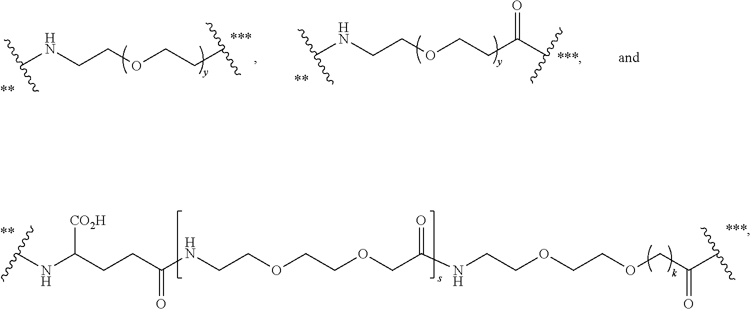
wherein:
y is an integer selected from 1 to 36,
s is 0, 1 or 2 and k is 1, 2 or 3, and
the wavy line marked ** indicate the attachment to the CO-group of formula (I), and
the wavy line marked *** indicate the attachment to group P.

4. A compound of formula (I) or a pharmaceutically acceptable salt thereof according to claim 3, wherein L is: 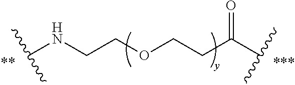
wherein y is an integer selected from 1 to 36.

5. A compound of formula (I) according to claim 1, which is a compound of formula (II) or a pharmaceutically acceptable salt thereof, 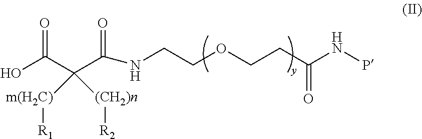
wherein:
NH—P′ represents a group P which is attached via a NH-moiety to the CO-group of linker L;
R₁ and R₂ are independently selected from CH₃, OH and CO₂H;
n and m are each an integer independently selected from 5 to 30;
and
y is an integer selected from 1 to 36.

6. A compound of formula (II) according to claim 5, wherein:
R₁ is CO₂H and R₂ is CH₃; n is 10 and m is 10;
R₁ is CO₂H and R₂ is CO₂H; n is 10 and m is 10;
R₁ is CO₂H and R₂ is CO₂H; n is 10 and m is 11;
R₁ is CO₂H and R₂ is CO₂H; n is 10 and m is 13; or
R₁ is CO₂H and R₂ is CO₂H; n is 10 and m is 14.

7. A compound of formula (II) according to claim 5, which is a compound of formula (III) or pharmaceutically acceptable salt thereof, 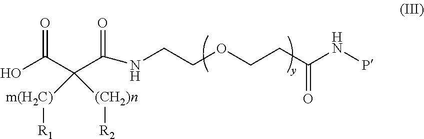
wherein R₁ is CO₂H and R₂ is CH₃.

8. A compound of formula (III) according to claim 7, which is a compound of formula (IV) or a pharmaceutically acceptable salt thereof, 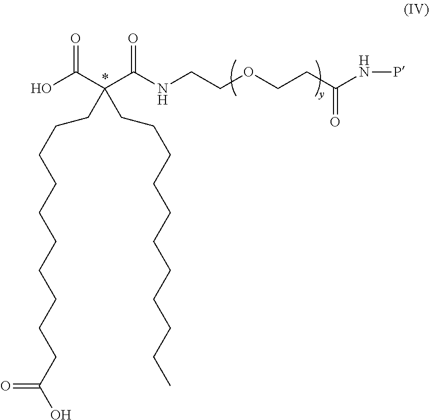
wherein the compound is present as a racemate, or as a stereochemically enriched mixture, or is stereochemically pure in respect of the carbon atom marked *.

9. A compound of formula (IV) according to claim 8, which is a compound of formula (IVa) or a compound of formula (IVb) or a pharmaceutically acceptable salt thereof, 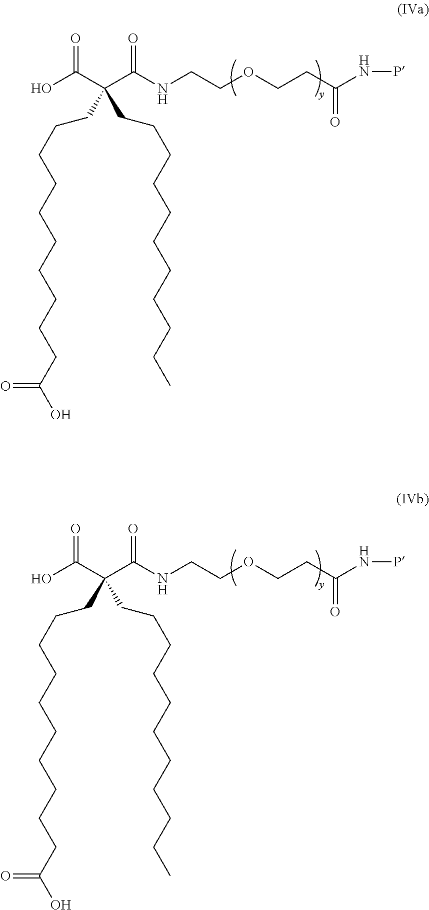
wherein y is an integer selected from 1 to 36.

10. A compound of formula (IVa) or a compound of formula (IVb) according to claim 9, wherein y is an integer selected from 2 to 24.

11. A compound of formula (IVa) or a compound of formula (IVb) according to claim 10, wherein y is 24.

12. A compound according to claim 1, or a pharmaceutically acceptable salt thereof, wherein P is selected from
GLP-1 (7-37): His-Ala-Glu-Gly-Thr-Phe-Thr-Ser-Asp-Val-Ser-Ser-Tyr-Leu-Glu-Gly-Gln-Ala-Ala-Lys-Glu-Phe-Ile-Ala-Trp-Leu-Val-Lys-Gly-Arg-Gly (SEQ ID NO:1), and
a GLP-1 analogue comprising a non-natural amino acid residue in position 7, or in position 8, or in position 7 and 8, relative to the sequence GLP-1 (7-37).

13. A compound of formula (I) according to claim 1, or a pharmaceutically acceptable salt thereof, wherein P is selected from:
[Aib8, Arg34]GLP-1 (7-37):
(SEQ ID NO: 3)
His-Aib-Glu-Gly-Thr-Phe-Thr-Ser-Asp-Val-Ser-Ser-
Tyr-Leu-Glu-Gly-Gln-Ala-Ala-Lys-Glu-Phe-Ile-Ala-
Trp-Leu-Val-Arg-Gly-Arg-Gly;
and
[Arg34]GLP-1 (7-37):
(SEQ ID NO: 4)
His-Ala-Glu-Gly-Thr-Phe-Thr-Ser-Asp-Val-Ser-Ser-
Tyr-Leu-Glu-Gly-Gln-Ala-Ala-Lys-Glu-Phe-Ile-Ala-
Trp-Leu-Val-Arg-Gly-Arg-Gly.

14. A compound of formula (I) according to claim 1, or a pharmaceutically acceptable salt thereof, wherein the P is [Aib8, Arg34]GLP-1 (7-37)
as shown below: 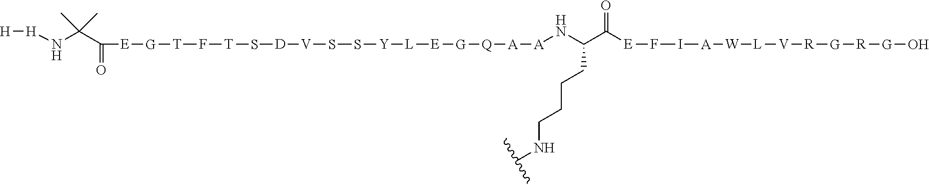
and wherein the wavy line on the amino-acid member Lys indicates the point of attachment to L.

15. A compound of formula (I) according to claim 1, or a pharmaceutically acceptable salt thereof, which is 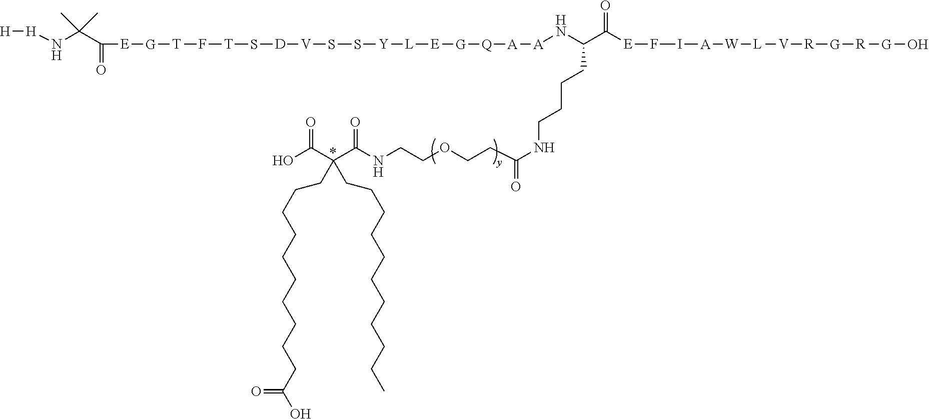
wherein y is an integer selected from 1 to 36, and
wherein the compound is present as a a diastereomeric mixture, a stereochemically enriched mixture or is stereochemically pure in respect of the carbon atom marked *.

16. A compound of formula (I) according to claim 1, or a pharmaceutically acceptable salt thereof, which is selected from:

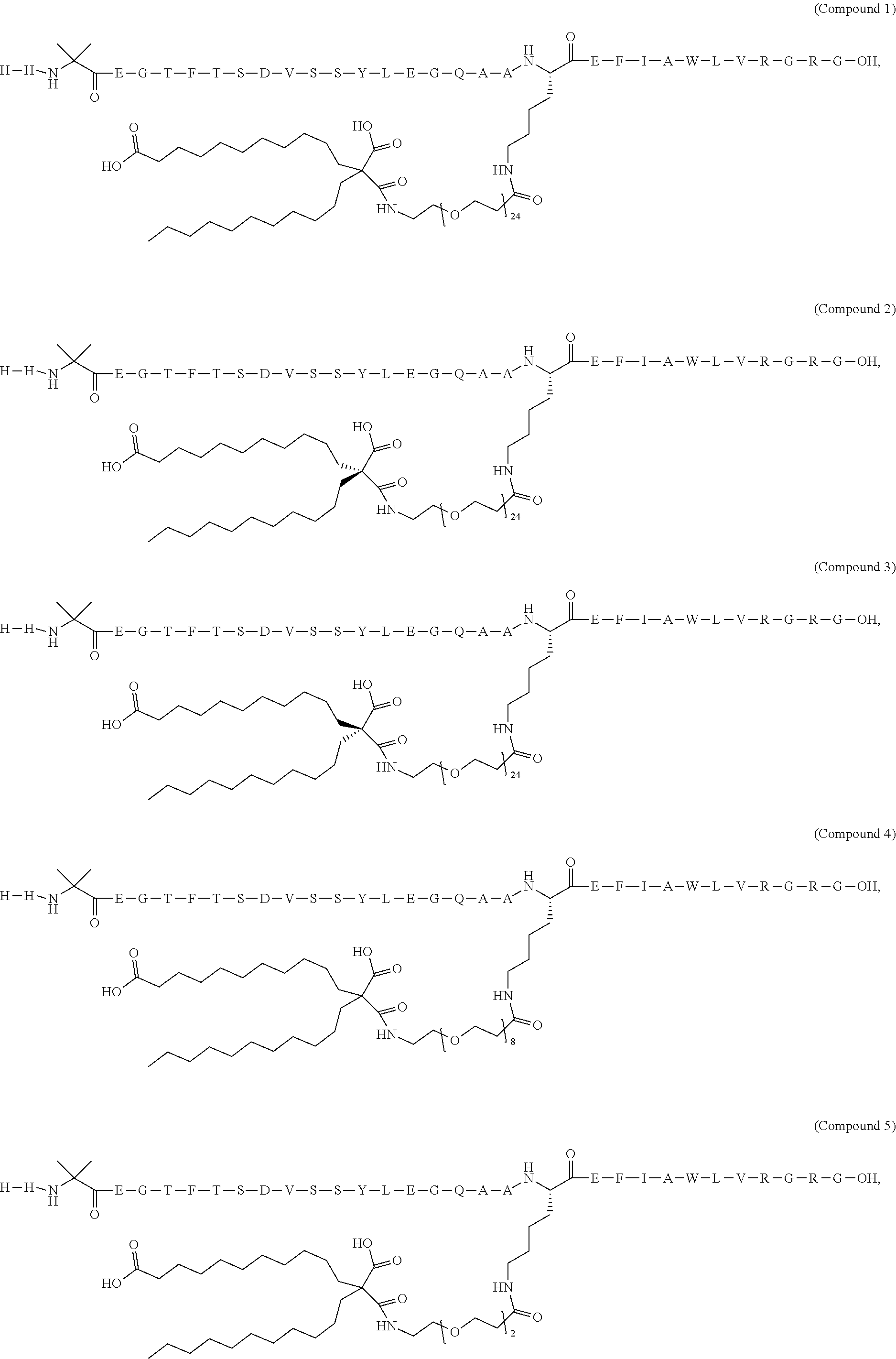

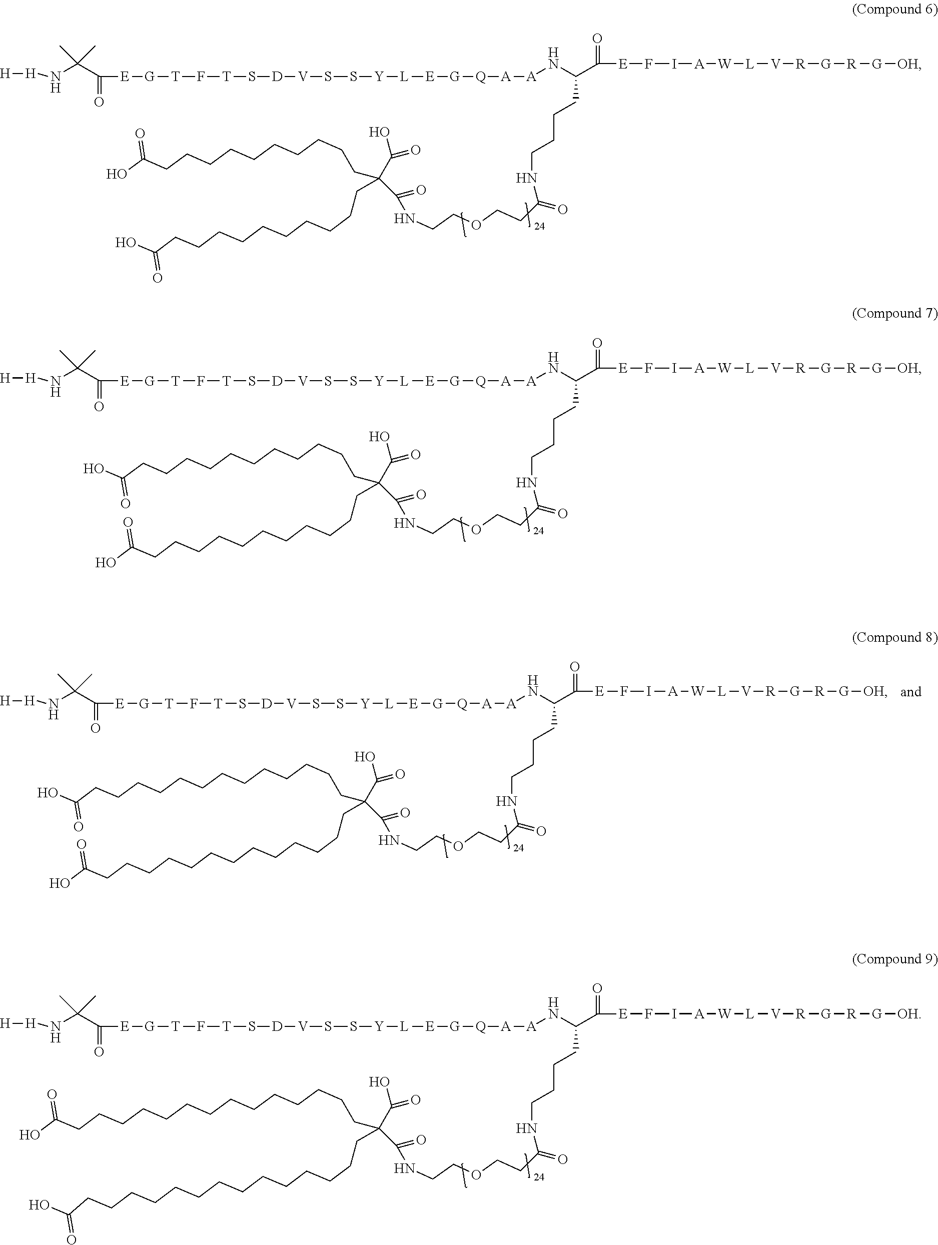

17. A compound of formula (I) according to claim 16, or a pharmaceutically acceptable salt thereof, which is:

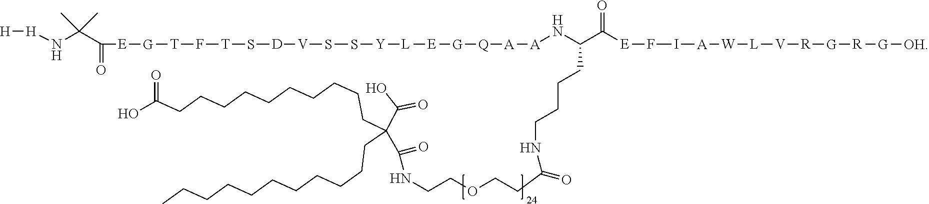

18. A compound of formula (I) according to claim 16, or a pharmaceutically acceptable salt thereof, which is:

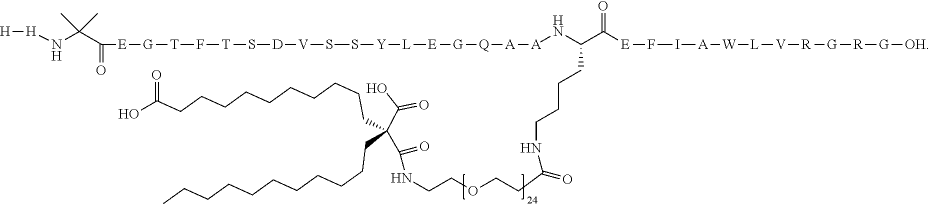

19. A compound of formula (I) according to claim 16, or a pharmaceutically acceptable salt thereof, which is:

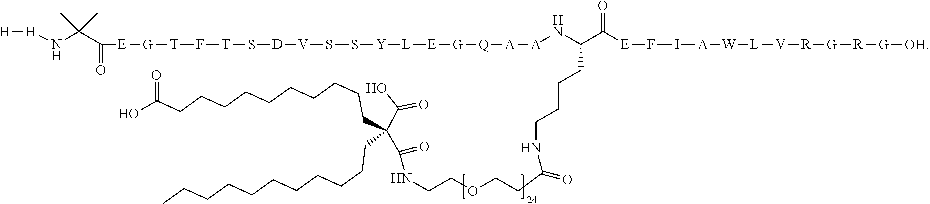

20. A pharmaceutical composition comprising a compound according to claim 1 or a pharmaceutically acceptable salt thereof and one or more pharmaceutically acceptable carriers.

21. A combination comprising a therapeutically effective amount of a compound according to claim 1 or a pharmaceutically acceptable salt thereof, and one or more therapeutically active agents.

22. A combination according to claim 21, wherein the compound is selected from Compound 1, 2 and 3.

23-26. (canceled)

27. A method for treating a patient in need of a therapy being susceptible to an agonist of the Glucagon-like Peptide 1 Receptor (GLP1R), comprising administering to the patient a therapeutically effective amount of a compound according to claim 1 or a pharmaceutically acceptable salt thereof.

28. A method of treating a disease or disorder selected from obesity, type 2 diabetes mellitus, insulin resistance, hyperinsulinemia, glucose intolerance, hyperglycemia, one or more diabetic complications selected from chronic kidney disease and diabetic nephropathy, dyslipidemia, metabolic syndrome, progressive liver disease selected from NAFLD and NASH, cardiovascular disease and peripheral neuropathy associated with diabetes, in a patient in need thereof, comprising administering to the patient a therapeutically effective amount of a compound according to claim 1 or a pharmaceutically acceptable salt thereof.

29. The method of claim 28, wherein the cardiovascular disease is selected from hypertension, atherosclerosis, peripheral arterial disease, stroke, cardiomyopathy, atrial fibrillation, heart failure selected from heart failure with reduced ejection fraction (HFrEF), heart failure with mid-range ejection fraction (HFmrEF) and heart failure with preserved ejection fraction (HFpEF), coronary heart disease and arrhythmias selected from atrial arrhythmias and ventricular arrhythmias.

30. The method according to claim 1, wherein the compound is selected from Compound 1, 2, and 3.
